SEQUENCE LISTING (10 sequences)

SEQ ID NO: 1 - protein, 470 aa, Artificial Sequence
  Synthetic
MGWSCIILFLVATATGVHSMDVKLVESGGGLVKPGGSLKLSCAASGFTFSSYTMSWVRQTPEKRLEWVATISSGGSYTYYPDSVKGRFTISRDKAKNTLYLQMGSLKSEDTAMYYCTRIGYDEDYAMDHWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 234 aa, Artificial Sequence
  TS2-16  kL-chain
MGWSCIILFLVATATGVHSEIVVTQSPTTMAASPGDKITITCSVSSIISSNYLHWYSQKPGFSPKLLIYRTSNLASGVPPRFSGSGSGTSYSLTIGTMEAEDVATYYCQQGSDIPLTFGDGTKLDLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 121 aa, Artificial Sequence
  Humanised TS2/16 H-chain
MDVKLVESGGGLVKPGGSLKLSCAASGFTFSSYTMSWVRQTPEKRLEWVATISSGGSYTYYPDSVKGRFTISRDKAKNTLYLQMGSLKSEDTAMYYCTRIGYDEDYAMDHWGQGTSVTVSS

SEQ ID NO: 4 - protein, 108 aa, Artificial Sequence
  Humanised TS2/16 L-chain
EIVVTQSPTTMAASPGDKITITCSVSSIISSNYLHWYSQKPGFSPKLLIYRTSNLASGVPPRFSGSGSGTSYSLTIGTMEAEDVATYYCQQGSDIPLTFGDGTKLDLK

SEQ ID NO: 5 - protein, 10 aa, Artificial Sequence
  Humanised TS2/16 H-chain HCDR1
GFTFSSYTMS

SEQ ID NO: 6 - protein, 17 aa, Artificial Sequence
  Humanised TS2/16 H-chain HCDR2
TISSGGSYTYYPDSVKG

SEQ ID NO: 7 - protein, 11 aa, Artificial Sequence
  Humanised TS2/16 H-chain HCDR3
IGYDEDYAMDH

SEQ ID NO: 8 - protein, 12 aa, Artificial Sequence
  Humanised TS2/16 L-chain LCDR1
SVSSIISSNYLH

SEQ ID NO: 9 - protein, 12 aa, Artificial Sequence
  Humanised TS2/16 L-chain LCDR2
SVSSIISSNYLH

SEQ ID NO: 10 - protein, 9 aa, Artificial Sequence
  Humanised TS2/16 L-chain LCDR2
QQGSDIPLT

(19) United States
(12) Patent Application Publication
(10) Pub. No.: US 2022/0340879 A1
(43) Pub. Date: Oct. 27, 2022

(54) IMPROVED CULTURE METHOD USING INTEGRIN AGONIST

(71) Applicant: Koninklijke Nederlandse Akademie van Wetenschappen, Utrecht (NL)

(72) Inventors: Willibrordus Barend Maria De Lau, Utrecht (NL); Johannes Carolus Clevers, Utrecht (NL)

(73) Assignee: Koninklijke Nederlandse Akademie van Wetenschappen, Utrecht (NL)

(21) Appl. No.: 17/611,561

(22) PCT Filed: May 18, 2020
(86) PCT No.: PCT/EP2020/063855

§ 371 Date: Nov. 15, 2021

(30) Foreign Application Priority Data
May 17, 2019 (GB) 1906978.0

(51) Int. Cl. C12N 5/071

(52) U.S. Cl. CPC C12N 5/0678; C12N 5/068; C12N 5/0689; C12N 2500/38; C12N 2501/11; C12N 2501/15; C12N 2501/415; C12N 2501/585; C12N 2513/00; C12N 2533/90

(57) ABSTRACT

The invention relates to improved methods for culturing an epithelial stem cell or an organoid comprising epithelial stem cells. The invention also relates to culture media suitable for use with said methods, organoids obtainable or obtained by said methods and uses of said culture methods, media and organoids in drug discovery and validation, toxicity assays, diagnostics and therapy.

### RELATED APPLICATIONS

This application is a national stage filing under 35 U.S.C. § 371 of International Patent Application No. PCT/EP2020/063855, filed May 18, 2020, which claims the benefit of British Patent Application No. 1906978.0, filed May 17, 2019, the contents of each of which are incorporated herein by reference in their entirety.

### REFERENCE TO A SEQUENCE LISTING SUBMITTED AS A TEXT FILE VIA EFS-WEB

The present application contains a Sequence Listing which has been submitted in ASCII format via EFS-Web and is hereby incorporated by reference in its entirety. Said ASCII copy, created on Nov. 15, 2021, is named C097370040US00-SEO-MAT and is 10,140 bytes in size.

### FIELD

The invention relates to in vitro cell culture methods for culturing stem cells or organoids. The invention relates to culture media suitable for use with said methods, organoids obtainable, or obtained by said methods and uses of said culture methods, media and organoids in drug discovery and validation, toxicity assays, diagnostics and therapy.

### BACKGROUND

Current methods for culturing stem cells or organoids in culture generally require the use of an extracellular matrix. Extracellular matrices consist of a multifunctional network of fibrous, gel-like material distributed throughout the body, which provide structural and biochemical support to all tissues. Matrix proteins have been implicated in many cellular processes including cell adhesion, proliferation, differentiation and apoptosis. Examples of extracellular matrix proteins include: laminins, collagens, glycoproteins, proteoglycans, and glycosaminoglycans, which self-assemble in the interstitial spaces between calls or as basement membranes. These extracellular matrix proteins can influence cell functions via a diverse range of cell receptors which can bind to some of the matrix components [1].

Examples of commercially available extracellular matrices, which can be used for culturing epithelial stem cells include basement membrane preparations from Engelbreth-Holm-Swarm (EHS) mouse sarcoma cells (e.g. Cultrex® Basement Membrane Extract (Trevigen, Inc.) or Matrigel™ (BO Biosciences)). However, the exact composition of these extracellular matrices and the mechanism by which they influence cellular functions remain unknown [2]. As the exact components of these extracellular matrices are not well-defined it can introduce a source of variability when culturing stem cells.

Therefore, there is a need in the art for synthetic replacements for these extracellular matrices, where the components of the matrix are controlled and reproducible. Synthetic matrices can be made from a variety of materials, such as chemically treated culture dish plastic, or layers of deposited protein, optionally supplemented with extracellular matrix proteins. However, synthetic matrices in the art only contain the major extracellular matrix proteins and are not applicable to all cell types [2]. Furthermore, whilst these synthetic matrices may support some stem cell growth, they are not yet as efficient as extracellular matrix.

Therefore there is a need in the art for improved methods of culturing stem cells. In particular, there, is a need for culturing methods which improve the growth of stem cells with a synthetic matrix.

### SUMMARY OF THE INVENTION

The invention provides methods for culturing an epithelial stem cell or an organoid comprising epithelial stem cells, wherein the method comprises culturing said epithelial stem cell in a culture medium suitable for epithelial stem cells, wherein the culture method further comprises contacting the cell or organoid with an integrin agonist. The inventors have advantageously identified that the use of an integrin agonist, such as an antibody, in methods for culturing an epithelial stem cell or an organoid results in improves cell growth in the presence or absence of an extra cellular matrix.

The invention further provides a culture medium for use in the methods of the invention which is suitable for epithelial stem and comprises one or more of a Wnt agonist, a BMP inhibitor, a mitogenic growth factor and a TGF-beta inhibitor for use in the methods of the invention. The invention also provides organoids obtainable or obtained by any of the methods of the invention.

Other aspects of the invention provide compositions comprising a culture medium suitable for epithelial stem as defined by the invention and an organoid obtainable or obtained by any of the methods of the invention. The invention also provides compositions comprising a culture medium suitable for epithelial stem as defined by the invention and an extracellular matrix or a synthetic matrix. The invention also provides an extracellular matrix or a synthetic matrix as defined by the invention, wherein the matrix further comprises an integrin agonist as defined in by the invention.

In further aspects, the invention provides uses of an integrin agonist as defined by the invention for culturing a cell. These include the use of an integrin agonist for pre-treating cells prior to transplantation into a subject and an integrin agonist for use as a cell adhesion-enhancer in a method of cell transplantation.

The invention also provides uses of the organoids of the invention for drug screening, target validation, target discovery or toxicology. In addition, the invention provides uses of the organoids of the invention for use in therapy or for use in diagnosis.

### BRIEF DESCRIPTION OF THE DRAWINGS

FIG. 1—The structure of integrin active and inactive forms [3].

FIG. 2—NUPAGE analysis of humanised TS2/16 antibody. Lane 1: pre strained marker, Lanes 2-4 show heavy chain to light chain constructs ratios of 1:1, 1:1.4 and 1:3 respectively.

FIG. 3—Immunofluorescence of K562 cells with humanised TS2/16

FIG. 4—Confirmation of the functional activity of humanized TS2/16 antibody

FIG. 5—The enhanced growth of epithelial stem cells using humanised TS2/16 without the presence of an extra cellular matrix.

FIG. 6—Examples of the growing organoids in the culture medium comprising the humanised TS2/16 antibody.

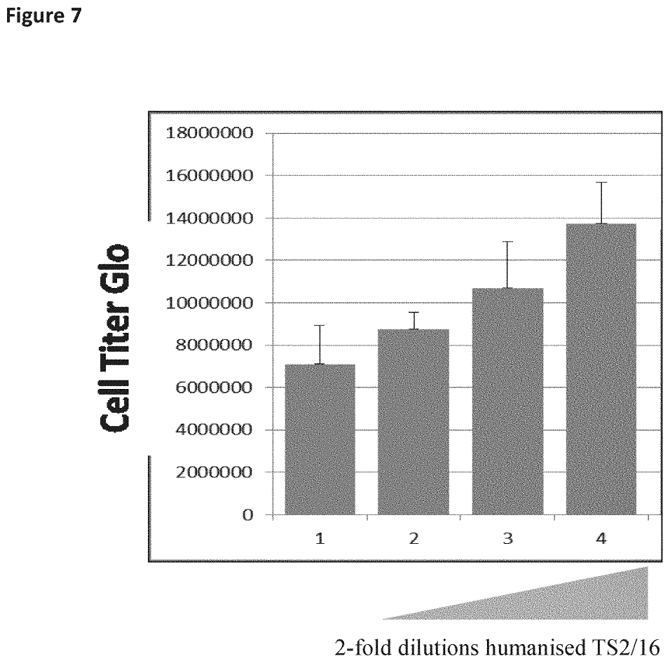

FIG. 7—The enhanced growth of epithelial stem cells using TS2/16 with Matrigel.

FIG. 8—The enhanced growth of epithelial stern cells using humanised TS2/16-Dyna beads enrichment of Matrigel. Bar 1 is treatment with humanised TS2/16 antibody and bar 2 is without treatment.

FIG. 9—The enhanced growth of epithelial organoid fragments (human colon) using humanised TS2/16 or mouse HUTS-4 Abs without the presence of an extracellular matrix. Bar 1 is without treatment, bar 2 is treatment with humanised TS2/16 antibody and bar 3 is treatment with HUTS-4 antibody.

FIG. 10—The enhanced growth of epithelial organoid fragments (human colon) using humanised TS2/16 without the presence of an extracellular matrix. Bar 1 is without treatment and bar 2 is treatment with humanised TS2/16 antibody.

FIG. 11—Examples of growing organoids in a culture medium comprising humanised TS2/16 antibody, HUTS-4 antibody and without an antibody. All culturing conditions were without Matrigel.

FIG. 12—Examples of the growing organoids initiated from fragments of an organoid in culture medium comprising humanised TS2/16 antibody (top) and in culture medium without the antibody.

FIG. 13—(A) The lack of epithelial stem cell growth in the presence of the AIIB2 antibody; (B) examples of organoids in culture medium comprising the humanised AIIB2 antibody (right) and in culture medium without the antibody (left).

FIG. 14—(A) The lack of epithelial stem cell growth in the presence of the AIIB2 antibody; (B) examples of organoids in culture medium comprising the humanised AIIB2 antibody (right) and culture medium without the antibody (left).

FIG. 15—(A) The lack of growth of epithelial stem cells in the presence of the AIIB2 antibody; (B) examples of organoids in culture medium comprising the humanised AIIB2 antibody (right) and in culture medium without the antibody (left).

FIG. 16—(A) Examples of growing pancreatic organoids in a culture medium comprising humanised TS2/16 antibody with Matrigel; (B) the enhanced growth of pancreatic epithelial stem cell using humanised TS2/16 with Matrigel.

FIG. 17—(A) The enhanced growth of lung epithelial stem cell using humanised TS2/16 with Matrigel; (B) examples of growing lung organoids in a culture medium comprising humanised TS2/16 antibody with Matrigel.

FIG. 18—(A)—Examples of growing head and neck organoids in a culture medium comprising humanised TS2/16 antibody with Matrigel; (B) the enhanced growth of head and neck epithelial stem cell using humanised TS2/16 with Matrigel.

FIG. 19—The lack of growth of epithelial stem cells in the presence of the Asci antibody.

FIG. 20—The synergistic effect of the pi integrin agonist TS/16 and the β4 integrin agonist 3E1 on epithelial stem cell growth. Bar 1 is a control; bar 2 is treatment with 0.1 μg/ml of TS2/16; bar 3 is treatment with 0.1 μg/ml of TS2/16 and 0.1 μg/ml of 3E1; bar 4 is treatment with 0.1 μg/ml of TS2/16 and 1 μg/ml of 3E1.

### DETAILED DESCRIPTION

As explained in detail in the Examples section, the inventors hypothesised that an anti-integrin antibody might be useful in the context of epithelial stem cell culture to mimic the signalling and the structural functions of an extracellular matrix. To test this hypothesis, the inventors generated an integrin agonist and tested this in a method for culturing epithelial stem cells in the absence of an extracellular matrix. The inventors surprisingly found that this improved epithelial stem cell growth and organoid formation in the absence of an extracellular matrix and increased growth efficiency so that it was more comparable to prior art methods Involving an extracellular matrix, such as Matrigel or BME. Moreover, they found that adding the integrin agonist also improved growth when included in a culture method involving an extracellular matrix. Thus surprisingly, the inventors showed that addition of an integrin agonist improved methods for culturing epithelial stem cells both with and without an extracellular matrix. Without wishing to be bound by theory, the inventors hypothesise that the integrin agonist activates signalling pathways and/or provides structural support that promote attachment of the epithelial stem cells to either the extracellular matrix or to a synthetic matrix material. In addition, the inventors hypothesise that the integrin agonist is able to mimic the structural and signalling function of the extracellular matrix, because the addition of an integrin agonist was surprisingly able to allow epithelial stem cell growth in the absence of the extracellular matrix. The ability of the integrin agonist to enhance epithelial stem cell growth in the absence of an extracellular matrix is particularly advantageous because it is a further step towards a defined, controlled and reproducible culture method.

Improving the growth rate of epithelial stem cells or organoids is also advantageous, because it enables a large number of cells to be available for various applications, for example, drug screening, in which a large amount of material is required to test various different drugs. The ability to generate the cells from a single starting source is advantageous for such applications where it is necessary to compare results between experiments. Similarly, it means that many cells ere available for use in transplants and that multiple patients may be transplanted with cells obtained from a useful donor. Culturing the cells in a culture medium allows the cells to multiply whilst retaining their stem sail phenotype. Organoids are formed comprising these stem cells. Use of the culture medium is therefore advantageous for providing increased numbers of these useful stem cells and for obtaining organoids containing these cells.

Therefore, the invention relates to methods of culturing epithelial stem cells or organoids comprising epithelial stem cells using an integrin agonist. In particular, the invention provides a method for culturing an epithelial stem cell or an organoid comprising epithelial stem cells, wherein the method comprises culturing said epithelial stem cell or organoid in a culture medium suitable for epithelial stem cells wherein the culture method further comprises contacting the cell or organoid with an integrin agonist.

### Integrin Agonist

Integrins are heterodimeric transmembrane adhesion receptors, which support cell-cell and cell-extracellular matrix interactions. They are formed by the non-covalent association of α and β subunits, which are present in all metazoans. Each subunit is a type I transmembrane glycoprotein that has a relatively large extracellular domain that mediates ligand binding and a short cytoplasmic tail (with the exception of the β4 subunit). Mammals have 18α and 8β subunits, which can combine to produce at least 24 different heterodimers, each with its own binding specificity [3] [9].

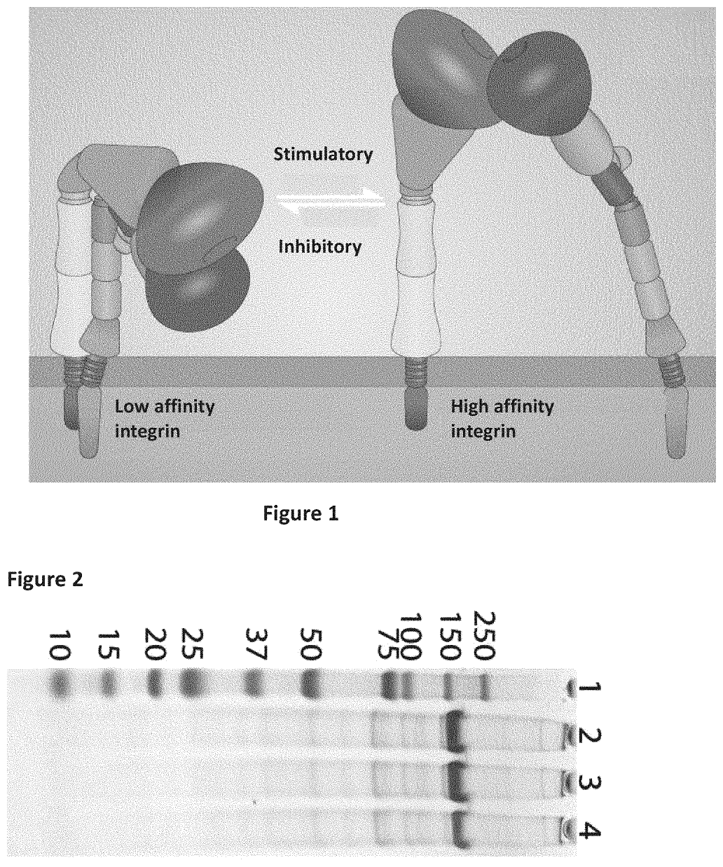

The structure of integrin is shown in FIG. 1 and includes a “head” region (which is the main point of contact between the two subunits), which is supported by two rod-like “legs”. Integrin adopts distinct conformations that have different binding affinities to integrin ligands. Examples of integrin ligands include collagen, laminin, thrombospondin and fibronectin [4]. In the bent-closed and extended-closed conformations the lower ‘legs’ and the transmembrane regions remain associated and the ligand-binding is closed. The adoption of a high-affinity extended open conformation involves a series of shape changes, including unbending of the receptor and various inter-module and intra-module movements. The shift from the bent-closed/extended-closed conformation to the extended-open conformation is termed ‘integrin activation’. Integrin activation can occur via the α or β subunit. Many anti-integrin antibodies that stimulate integrin activation and ligand binding (including TS2/16) function allosterically. Antibody binding shifts the conformational equilibrium of integrin from an inactive to an active form by stabilising the active integrin signalling conformation forms. The result is therefore an increase in proportion of active integrity molecules [5]. Studies investigating the β1 integrin subfamily have shewn that TS2/16, 12G10, and HUTS-4 Fabs antibodies induce nearly identical high affinities for cyclic RGD peptide which suggests they stabilize identical conformations of the ligand-binding site in the β1 domain [6].

Therefore, integrin agonists are agents which activate integrin. In other words, integrin agonist are agents which induce a conformational change in integrin, and thus increases its binding affinity to an integrin ligand. In some embodiments, the integrin agonist is not collagen, laminin, thrombospondin or fibronectin. In some embodiments, the integrin agonist activates integrin and also mimics the activity of an integrin ligand binding to integrin. In some embodiments, the integrin agonist can activate integrin independent of the conformational state of integrin, and also mimic the activity of an integrin ligand binding to integrin.

These integrin agonists are expected to be particularly useful in the context of the invention integrin agonists can be easily identified by the skilled person using the cell adhesion assay as described in Example 3 and in reference [17]. Integrin agonists that can also mimic the activity of a ligand binding to integrin can be identified by the skilled person using the assay as described in Example 4.

In some embodiments, the integrin agonist has the same or similar affinity for cyclic RGD peptide as the integrin agonist TS2/16. For example, the integrin agonist binds to FITC-cRGD in a Fluorescence Polarization assay as described in [7] with an FPₘₐₓ of 0.2-0.3, 0.20-0.25 or 0.21-0.23 or more preferably with an FPₘₐₓ of 0.21-0.22. In some embodiments, the integrin agonist can stabilize the same open headpiece conformation of integrin as TS2/16.

Integrin agonists that have an affinity for cyclic RGD peptide that is similar to or the same as the integrin agonist TS2/16 can be identified by the skilled person using the assay as described in [7]. Integrin agonists that can stabilize the same open headpiece conformation of integrin as TS2/16 can be identified by the skilled person using the electron microscopy assay described in reference [6].Many integrin agonists are well known in the art, including anti-integrin antibodies, talins, kindlins, reducing agents such as dithiothreitol and lipids such as 25-hydroxycholesterol [8-11]. In preferred embodiments, the integrin agonist activates integrin through its extracellular domain. Such integrin agonists include anti-integrin antibodies and reducing agents, such as dithiothreitol and lipids such as 25-hydroxycholesterol. Talin and kindlins, by contrast, activate integrin through interactions with the intracellular domains of integrin.

Antibodies directed to integrin can either have activating or inhibitory properties. The term integrin agonists encompasses only anti-integrin antibodies which have stimulatory or activating effect. All stimulatory or activation-specific anti-integrin monoclonal antibodies appear to increase ligand-binding affinity by reducing the rate of dissociation and can be broken down into two classes. The first subclass recognises epitopes that that are regulated by ligand and cation binding (known as ligand-induced binding sites “LIBS”), such as the anti-β1 monoclonal antibody HUTS-4, while the second subclass is not affected by ligand or cation binding (which includes the anti-1 monoclonal antibody, TS2/16).

Examples of activating anti-integrin antibodies include:

TABLE 1
Summary of stimulatory anti-integrin antibodies

| Integrin subunit | Antibody |
|---|---|
| α2 | JBS2 |
| α4 | HP1/3 |
| α5 | SNAKA51 |
| α11b | PTS25-2, PMI-1 |
| αL | MEM-83, NKI-L16 |
| αX | 496B |
| β1 | 12G10, 8A2, TS2/16, 15/7, |
| | HUTS-4, 8E3, N29, 9EG7 |
| β2 | mAb 24, MEM-148, KIM127, CBR |
| | LFA-1/2, MEM-48, KIM185 |
| β4 | 3E1 |
| β3 | AP3, AP5, LIBS6, LIBS2 |
| β7 | 10F8, 2B8, 2G3 |

The inventors have demonstrated that the addition of an integrin agonist to cell culture medium can improve the growth of epithelial stem cells without the need for an extracellular matrix. The examples show that the addition of a humanised TS2/16 anti-integrin antibody to cell culture medium improved the growth of epithelial stem cells without the need for an extracellular matrix. Furthermore, the inventors have demonstrated that addition of an integrin agonist can improve the growth of epithelial stem cells in combination with the extracellular matrix is Matrigel (a Basement Membrane extract). Therefore integrin agonists as described herein are particularly useful for culturing epithelial stem cells.

In some embodiments, the integrin agonist is selected from an anti-integrin antibody, a talin, a kindlin, dithiothreitol and oxysterol 25-hydroxycholesterol.

Without wishing to be bound by any particular theory, an integrin agonist can advantageously improve the growth of epithelial stem cells without the need for an extracellular matrix, because the integrin agonist can induce a conformational state of integrin that is normally associated with ligand binding The integrin agonist can induce a high affinity conformation of integrin and activate signalling pathways that are normally activated by ECM components. The examples demonstrate that anti-integrin antibodies which are regulated by ligand and cation binding (such as HUTS-4), and those that are not affected by ligand or cation binding (such as TS2/16) can both be particularly useful for culturing epithelial stem cells. The integrin agonist can interact via the alpha or the beta subunits of integrin, for example by interacting with the α2, α4, α5, α11b, αL, αX, β1, β2, β3, β4 or β7. In some embodiments, the integrin agonist interacts with beta the subunits β1, β2, β3, β4 or β7. In preferred embodiments, the integrin agonist interacts with the β1 subunit.

The integrin agonist can be an anti-integrin antibody. For the avoidance of any doubt, the anti-integrin antibodies of the invention are stimulatory anti-integrin antibodies. Examples of anti-integrin antibodies include JBS2, HP1/3, SNAKA51, PTS25-2, PMI-1, MEM-83, NKI-L16, 496B, 12G10, 8A2, TS2/16, 15/7, HUTS-4, 8E3, N29, 9EG7, mAb 24, MEM-148, KIM127, CBR LFA-1/2, MEM-48, KIM185, AP3, AP5, LIBS6, LIBS2, 10F8, 2B8 and 2G3 antibodies. In some embodiments, the anti-integrin antibody interacts with the β1 subunit, such as TS2/16, 12G10, 8A2, 15/7, HUTS-4, 8E3, N29 and 9EG7 antibodies. In one embodiments, the anti-integrin antibody interacts with the β1 subunit and is MAB1778 (Catalogue number: #MAB1778, which is available at this website rndsystems.cm/products/human-integrin-beta1-cd29-antibody-4b7r_mab1778).

TS2/16, 12G10 or 8A2 antibodies are known to interact with the βA domain. The examples demonstrate fie advantageous properties of a TS2/16 anti-integrin antibody for culturing epithelial stem cells. Therefore, the anti-integrin antibodies of the invention can preferably interact with the βA domain. In preferred embodiments, the anti-integrin antibody is TS2/16, 12G10 or 8A2. In other preferred embodiments, the anti-integrin antibody is TS2/16, 12G10 or HUTS-4. In further preferred embodiments, the anti-integrin antibody is TS2/16 (or a humanised TS2/16) or HUTS-4 (or a humanised HUTS-4). In further preferred embodiments the anti-integrin antibody is TS2/16 or a humanized TS 2/16. In further preferred embodiments the anti-integrin antibody is HUTS -4 or a humanised HUTS-4. A summary of known activating anti-integrin β1 chain antibody epitopes is provided in Table 2. These epitopes fall into two groups; the first consists of a very short sequence of residues located in the predicted ligand-binding domain (residues 207-218), and the other has an epitope in or near the cysteine-rich repeats (residues 442-629) located in the membrane proximal stalk-like region of the integrin. Some activating anti-integrin β1 chain antibodies exert an effect only when the integrin has a particular conformation and it is thought that these antibodies bind to epitopes that are only exposed under certain physiological conditions [13], such as HUTS-4, HUTS-7 and HUTS-21 [12].

TABLE 2
Summary of activating anti-integrin antibodies [13] and [12]

| Activating Antibody | Epitope location | Conformation dependence |
|---|---|---|
| TS2/16 | 207-218 | No |
| 8A2 | 207-218 | No |
| A1A5 | 207-218 | No |
| AG89 | 426-587 | Yes |
| 15/7 | 355-425 | Yes |
| HUTS-4 | 355-425 | Yes |
| HUTS-7 | 355-425 | Yes |
| HUTS-21 | 355-425 | Yes |
| 9EG7 | 495-602 | Yes |
| QE.2E5 | 426-587 | n.d. |
| TASC (chicken β1) | 304-602 | n.d. |
| JB1B | 671-703 | n.d. |
| B3B11 | 657-670 | n.d. |

Therefore, in some embodiments the integrin agonist (optionally the anti-integrin antibody) interacts with at least a portion of residues 207-21 8 on the β1 subunit. In some embodiments the integrin agonist (optionally the anti-integrin antibody) interacts with at least a portion of residues 442-629 on the β1 subunit. In some embodiments the integrin agonist (optionally the anti-integrin antibody) interacts with at least a portion of residues 335-425 on the β1 subunit. In some embodiments, integrin agonists that bind independent of the conformation of the integrin are preferred, because their ability to activate integrin is not dependent on the physiological conditions. In preferred embodiments, the epitope for the integrin antibody is not a ligand-induced binding site. The anti-integrin antibody can comprise a heavy chain variable region (VH) of SEQ ID NO: 1 and a light chain variable region (VL) of SEQ ID NO: 2.

The anti-integrin antibody can also be humanised, for example the anti-integrin antibody can be humanised TS2/16 antibody as demonstrated in the examples. The humanised TS2/16 antibody can comprise a heavy chain variable reason (VH) of SEQ ID NO: 3 and a light chain variable region (VL) of SEG ID NO: 4. In some embodiments, the anti-integrin antibody comprises the heavy chain CDRs HCDR1: HCDR2 and HCDR3 of SEQ ID NOs: 5, 6 and 7 respectively, and light chain CDRs LCDR1, LCDR2 and LCDR3 of SEQ ID NOs: 8, 9 and 10 respectively.

The examples demonstrate the advantageous and synergistic properties of an agonistic anti-β1 integrin anybody (TS2/16) in combination with an agonistic anti-β4 antibody (3E1) for culturing epithelial stem cells. Therefore, the integers agonists can be used in combination to culture epithelial stem cells. For example, multiple anti-integrin antibodies can be used to culture epithelial stem cells. In some embodiments the two anti-integrin antibodies target the same subunit of integrin, for example 2 or more β1 integrin agonist antibodies are used to culture epithelial stem cells. In some embodiments, the two or more β1 integrin agonist antibodies are selected from TS2/16, 12G10, 8A2, 15/7, HUTS-4, 8E3, N29 and 9EG7, or are preferably selected from TS2/16, 12G10 and 8A2. In some embodiments, TS2/16 is used in combination with HUTS-4.

Alternatively the two anti-integrin antibodies target different subunits of integrin. Possible combinations of anti-integrin antibodies include: (1) a β1 integrin agonist antibody in combination a β2 integrin agonist antibody; (2) a β1 integrin agonist antibody in combination a β3 integrin agonist antibody; (3) a β1 integrin agonist antibody in combination a β4 integrin agonist antibody; (4) a β1 integrin agonist antibody in combination a β7 integrin agonist antibody; (5) a β1 integrin agonist antibody in combination a α2, integrin agonist antibody: (6) a β1 integrin agonist antibody in combination a α4 integrin agonist antibody; (7) a β1 integrin agonist antibody in combination a α5 integrin agonist antibody; (8) a β1 integrin agonist antibody in combination a α11b integrin agonist antibody; (9) a β1 integrin agonist antibody in combination a αL integrin agonist antibody or (10) a β1 integrin agonist antibody in combination a αX integrin agonist antibody.

In preferred embodiments, a β1 integrin agonist antibody is used in combination a β4 integrin agonist antibody to culture epithelial stem cells. In preferred embodiments the β1 integrin agonist antibody is TS2/16, 12G10 or 8A2. In preferred embodiments the p4 integrin agonist antibody is 3E1 in further preferred embodiments, TS2/16 and 3E1 are used in combination to culture epithelial stem cells.

In some embodiments, a β1 integrin agonist antibody is used in combination with a β4 integrin agonist antibody and an agonistic anti-integrin antibody that binds to one of α2, α4, α5, α11b, αL, αX, β2, β3 and β7.

In some embodiments, a β1 integrin agonist antibody is used in combination with: (i) a β4 integrin agonist antibody and (ii) two or more (e.g. three, four, five, six, seven, eight or nine) integrin agonist antibodies, each of which binds to a different integrin subunit selected from: α2, α4, α5, α11b, αL, αX, β2, β3 and β7.

In some embodiments, a β2 integrin agonist antibody is used in combination with an agonistic anti-integrin antibody that binds to one of α2, α4, α5, α11b, αL, αX, β1, β3, β4 and β7. In some embodiments, a β2 integrin agonist antibody is used In combination with two or more (e.g. three, four, five, six, seven, eight, nine or ten) integrin agonist antibodies, each of which binds to a different integrin subunit selected from α2, α4, α5, α11b, αL, αX, β1, β3, β4 and p7.

In some embodiments, a β3 integrin agonist antibody is used in combination with an agonistic anti-integrin antibody that binds to one of α2, α4, α5, α11b, αL, αX, β1, β2, β4 and β7. In some embodiments, a β3 integrin agonist antibody is used in combination with two or more (e.g. three, four, five, six, seven, eight, nine or ten) integrin agonist antibodies, each of which binds to a different integrin subunit selected from α2, α4, α6, α11b, αL, αX, β1, β2, β4 and β7.

In some embodiments, a β4 integrin agonist antibody is used in combination with on agonistic anti-integrin antibody that binds to one of α2, α4, α5, α11b, αL, αX, β1, β2, β3 and β7. In some embodiments, a β4 integrin agonist antibody is used in combination with two or more (e.g. three, four, five, six, seven, eight, nine or ten) integrin agonist antibodies, each of which binds to a different integrin subunit selected from α2, α4, α5, α11b, αL, αX, β1, β2, β3 and β7.

In some embodiments, a β7 integrin agonist antibody is used in combination with an agonistic anti-integrin antibody that binds to one of α2, α4, α5, α11b, αL, αX, β1, β2, β3 and β4. In some embodiments, a β7 integrin agonist antibody is used in combination with two or more (e.g. three, four, five, six, seven, eight, nine or ten) integrin agonist antibodies, each of which binds to a different integrin subunit selected from α2, α4, α5; α11b, αL, αX, β1, β2, β3 and β4.

In some embodiments, an α2 integrin agonist antibody is used in combination with an agonistic anti-integrin antibody trial binds to one of α4, α5, α11b, αL, αX, β1, β2, β3, β4 and β7. In some embodiments, an α2 integrin agonist antibody is used in combination with two or more (e.g. three, four, five, six, seven, eight, nine or ten) integrin agonist antibodies, each of which binds to a different integrin subunit selected from: α4, α5, α11b, αL, αX, β1, β2, β3, β4 and β7.

In some embodiments, an α4 integrin agonist antibody is used in combination with an agonistic anti-integrin antibody that binds to one of α2, α5, α11b, αL, αX, β1, β2, β3, β4 and β7. In some embodiments, an α4 integrin agonist antibody is used in combination with two or more (e.g. three four, five, six, seven, eight, nine or ten) integrin agonist antibodies, each of which binds to a different integrin subunit selected from: α2, α5, α11b, αL, αX, β1, β2, β3, β4 and β7.

In some embodiments, an α5 integrin agonist antibody is used in combination with an agonistic anti-integrin antibody that binds to one of α2, α4, α11b, αL, αX, β1, β2, β3, β4 and β7. In some embodiments, an α5 integrin agonist antibody is used in combination with two or more (e.g. three, four, five, six, seven, eight, nine or ten) integrin agonist antibodies, each of which binds to a different integrin subunit selected from: α2, α4, α11b, αL, αX, β1, β2, β3, β4 and β7.

In some embodiments, an a11b integrin agonist antibody is used in combination with an agonistic anti-integrin antibody that binds to one of α2, α4, α5, αL, αX, β1, β2, β3, β4 and β7. In some embodiments, an α11b integrin agonist antibody is used in combination with two or more (e.g. three, four, five, six, seven, eight, nine or ten) integrin agonist antibodies, each of which binds to a different integrin subunit selected from: α2, α4, α5, αL, αX, β1, β2, β3, β4 and β7.

In some embodiments, an αL integrin agonist anybody is used in combination with an agonistic anti-integrin antibody that binds to one of α2, α4, α5, α11b, αX, β1, β2, β3, β4 and β7. In some embodiments, an αL integrin agonist antibody is used in combination with two or more (e.g. three, four, five, six, seven, eight, nine or ten) integrin agonist antibodies, each of which binds to a different integrin subunit selected from: α2, α4, α5, α11b, αX, β1, β2, β3, β4 and β7.

In some embodiments, an αX integrin agonist antibody is used in combination with an agonistic anti-integrin antibody that binds to one of α2, α4, α5, α11b, αL, β1, β2, β3, β4 and β7. In some embodiments, an αX integrin agonist antibody is used in combination with two or more (e.g. three, four, five, six, seven, eight, nine or ten) integrin agonist antibodies, each of which binds to a different integrin subunit selected from: α2, α4, α5, α11b, αL, β1, β2, β3, β4 and β7.

In other preferred embodiments, integrin agonists whose binding is dependent of the conformation of the integrin. In other embodiments, the epitope for the integrin antibody is a ligand-induced binding sites. The anti-integrin antibody can be HUTS-4. In some embodiments, the anti-integrin antibody binds to the same epitope as the TS2/16 antibody. Accordingly, in some embodiments, the anti-integrin antibody binds to the same epitope as an antibody comprising a heavy chain variable region (VH) of SEQ ID NO: 3 and a light chain variable region (VL) of SEQ ID NO: 4. In some embodiments, the anti-integrin antibody binds to the same epitope as the HUTS-4 antibody.

In some embodiments, the anti-integrin antibody competes for binding to the β1 subunit with the TS2/16 antibody. Accordingly, in some embodiments, the anti-integrin antibody binds to the same epitope as an antibody comprising a heavy chain variable region (VH) of SEQ ID NO: 3 and a light chain variable region (VL) of SEQ ID NO: 4. In some embodiments, the anti-integrin antibody competes for binding to the β1 subunit with the HUTS-4 antibody. In some embodiments, the anti-integrin antibody competes for binding to the β4 subunit with the 3E1 antibody.

A conventional antibody is comprised of two identical heavy chains and two identical light chains that are joined by disulfide bonds. Each heavy and light chain contains a constant region and a variable region. Each variable region contains three CDR3 which are primarily responsible for binding an epitope of a target antigen. They are referred to as CDR1, CDR2, and CDR3, numbered sequentially from the N-terminus, of which the CDR3 region comprises the most variable region and normally provides a substantial part of the contact residues to a target. The more highly conserved portions of the variable regions are called the “framework regions”. In some embodiments, the antibody portion comprises at least one heavy chain and at least one light chain. In some embodiments, the antibody portion consists of one heavy and one light chain.

The term antibody is used herein in the broadest sense and specifically covers, but is not limited to: monoclonal antibodies (including full length monoclonal antibodies) of any isotype such as IgG, IgM, IgA, IgD and IgE, polyclonal antibodies including recombinant polyclonal antibodies, Oligoclonics, multispecific antibodies, chimeric antibodies, nanobodies, diabodies, BiTE's, Tandabs, mimetobodies, bispecific antibodies, humanized antibodies, human antibodies, deimmunised antibodies and antibody fragments. In addition, scaffolds will be covered under this term, such as Acticalins, Ankarins, etc. An antibody reactive with a specific epitope of the target molecule can be generated by recombinant methods such as selection of libraries of recombinant antibodies in phage or similar vectors, or by immunizing an animal with the target nucleic acid encoding the target epitopes.

In certain embodiments, the antibodies or antigen-binding fragments of the present invention are bispecific comprising a first binding specificity to a first epitope in an integrin subunit and a second binding specificity to a second epitope in an integrin subunit, wherein the first and second epitopes are distinct and non-overlapping. In certain embodiments, the bispecific antibody binds different epitopes on the same integrin subunit, for example the β1 subunit. In other embodiments, the bispecific antibody binds epitopes on different integrin subunits, for example the bispecific antibody has a first binding specificity to a first epitope in a β1 integrin subunit and a second binding specificity to a second in a β3 integrin subunit.

In some embodiments, an antibody according to the invention consists of or comprises a single domain antibody (also termed a sdAb or Nanobody), a F(ab′)2, Fab, Fab′, Facb, or single chain Fv (scFv) fragment. A scFv fragment is an epitope-binding fragment that contains at least one fragment of an antibody heavy chain variable region (VH) linked to at least one fragment of an antibody light chain variable region (VL). The linker may be a short, flexible peptide selected to assure that the proper three-dimensional folding of the VL and VH regions occurs once they are linked so as to maintain the target molecule binding-specificity of the whole antibody from which the single-chain antibody fragment is derived. The carboxyl terminus of the VL or VH sequence may be covalently linked by a linker to the amino acid terminus of a complementary VL or VH sequence.

In some embodiments, the antibody portion of the fusion protein comprises a constant region or Fc region. Fc regions may be obtained from native forms isolated from humans and other animals including cows, goats, swine, mice, rabbits, hamsters, rats and guinea pigs, or may be recombinants or derivatives thereof, obtained from transformed animal calls or microorganisms. They may be obtained from a native immunoglobulin by isolating whole immunoglobulins from human or animal organisms and treating them with a proteolytic enzyme. Papain digests the native immunoglobulin into Fab and Fc regions, and pepsin treatment results in the production of pF′c and F(ab)2 fragments. These fragments may be subjected, for example, to size-exclusion chromatography to isolate Fc.

In some embodiments, the fc region is modified. For example, the immunoglobulin Fc region of the present invention may be in the form of having native sugar chains, increased sugar chains compared to a native form or decreased sugar chains compared to the native form, or may be in a deglycosylated form. The increase, decrease or removal of the immunoglobulin Fc sugar chains may be achieved by methods common in the art, such as a chemical method, an enzymatic method and a genetic engineering method using a microorganism. The removal of sugar chains from an Fc region results in a sharp decrease in binding affinity to the complement (c1q) and a decrease or loss in antibody-dependent cell-mediated cytotoxicity (ADCC) or complement-dependent cytotoxicity (CDC), hereby not inducing unnecessary immune responses in vivo. In this regard, an immunoglobulin Fc region in a deglycosylated or aglycosylated form (the latter produced for example by a prokaryote, preferably E. coli) may be used according to the present invention. In addition, the immunoglobulin Fc region may be an fc region that is derived from IgG, IgA, IgD, IgE and IgM, or that is made by hybrids thereof (sequences encoding two or more immunoglobulin Fc regions of different origin, present in a single-chain immunoglobulin Fc region). In the present invention, various types of hybrids are possible. That is, domain hybrids may be composed of one to four domains selected from the group consisting of CH1, CH2, CN3 and CH4 of IgG Fc, IgM Fc, IgA Fc, IgE Fc and IgD Fc, and may include a hinge region. Therefore, in some embodiments, the Fc region is a hybrid. Preferably, the Fc region is derived from IgG or IgM, which are among the most abundant proteins in the human blood, and most preferably from IgG, which is known to enhance the half-life of ligand-binding proteins. Further, IgG is divided into IgG1, IgG2, IgG3 and IgG4 subclasses, and the present invention includes combinations or hybrids thereof. Preferred are IgG2 and IgG4 subclasses.

In some embodiments, the Fc region is IgG in isotype, for example IgG1, preferably human IgG, or human IgG1.

In some embodiments, the fc region is modified to silence or reduce ADCC and/or complement effector functions. In some embodiments, the Fc region is modified for increased FcRn affinity (which can extend half-life). Suitable modifications are described in Monnet, Céline, et al., “Selection of IgG Variants with Increased FcRn Binding Using Random and Directed Mutagenesis Impact on Effector Functions.” Frontiers in Immunology 6:39 (2015). In some embodiments, the antibody contains an Fc domain or a portion thereof that binds to the FcRn receptor. As a non-limiting example, a suitable Fc domain may be derived from an immunoglobulin subclass such as IgA, IgE, IgG or IgM. In some embodiments, a suitable Fc domain is derived from IgG1, IgG2, IgG3, or IgG4. Particularly suitable Fc domains include those derived from human antibodies.

In some embodiments, the modified Fc region is an IgG variant with one or more, or preferably all, of the mutations. E294Del, T307P, and N434Y (e.g. see Table 6, entry “C6A-66” of Monnet et al.,). These particular mutations are expected to silence ADCC and complement effector functions and increase half-life.

In some embodiments, the antibody of the invention is a polyclonal, monoclonal, multispecific, mouse, human, humanized, primatized or chimeric antibody or a single-chain antibody. In some embodiments, antibodies may be human or humanized antibodies. In a preferred embodiment, the anybody is a humanized antibody.

In some embodiments, the antibody binds to integrin with a K_{D} of 10⁻⁷ M or less, 10⁻⁸ M or less, 10⁻⁹ M or less, or 10⁻¹⁰ M or less. For example, the antibody can bind to the α or β subunit of integrin, such as α2, α4, α5, α11b, αL, αX, β1, β2, β3, β4, and β7. In some embodiments, the antibody binds to the subunits β1, β2, β3, β4 or β7 of integrin and in preferred embodiments, the antibody binds to the β1 subunit of integrin. Accordingly, in a preferred embodiment, the antibody binds to the β1 subunit of integrin with a K_{D} of 10⁻⁷ M or less, 10⁻⁸ M or less, 10⁻⁹ M or less, or 10⁻¹⁰ M or less. In a further embodiment, a first antibody that binds to the β1 subunit of integrin with a K_{D} of 10⁻⁷ M or less, 10⁻⁸ M or less, 10⁻⁹ M or less, or 10⁻¹⁰ M or less is combined with a second antibody that binds to the β4 subunit of integrin with a K_{D} of 10⁻⁷ M or less, 10⁻⁸ M or less, 10⁻⁹ M or less, or 10⁻¹⁰ M or less. For example, in one embodiment, a first antibody that binds to the β1 subunit of integrin with a K_{D} of 10⁻⁸ M or less is combined with a second antibody that binds to the β4 subunit of integrin with a K_{D} of 10⁻⁸ M or less.

In some embodiments, antibody binding affinity is determined using an Octet® RED96 system (ForteBio, Inc.). For example, a Flag-tagged β1 subunit, a Flag-tagged β2 subunit a Flag-tagged β3 subunit or a Flag-tagged β7 subunit may be immobilized to an anti-Flag biosensor and incubated with varying concentrations of the antibody in solution, binding data are then collected. In some embodiments, antibody binding affinity is determined by surface plasmon resonance.

In some embodiments, whether a test antibody competes with a reference antibody for binding to integrin is determined using an in vitro binding competition assay. For example, a Flag-tagged β1 subunit, a Flag-tagged β2 subunit, a Flag-tagged β3 subunit or a Flag-tagged β7 subunit may be immobilized to an anti-Flag biosensor, the association of the reference antibody to the immobilized Flag-tagged β1, β2, β3 or β7 subunit is then measured (e.g. using the Octets RED96 system, ForteBio, Inc.) and then the degree of additional binding is assessed by exposing the immobilized Flag-tagged β1, β2, β3 or β7 subunit to the test antibody in the presence of the reference antibody. In some embodiments, the anti-integrin antibody is a heavy chain-only antibody. The term “antibody” encompasses entire tetrameric antibodies and antigen-binding fragments thereof. In some embodiments, the antigen-ending fragment thereof is selected from a VH domain, Fab, Fab′, F(ab′)2, Fd, Fv, a single-chain Fv (scFv) and a disulfide-linked Fv (sdFv).

An antigen-binding fragment of an anybody will typically comprise at least one variable domain. The variable domain may be of any size or amino acid composition and will generally comprise at least one CDR, which is adjacent to or in frame with one or more framework sequences. In antigen-binding fragments having a VH domain associated with a V_{L} domain, the VH and V_{L} domains may be situated relative to one another in any suitable arrangement. For example, the variable region may be dimeric and contain VH-VH, VH-V_{L} or V_{L}-V_{L} dimers. Alternatively, the antigen-binding fragment of an antibody may contain a monomeric VH or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) VH-C_{H}1; (ii) VH-C_{H}2; (iii) VH-C_{H}3; (iv) VH-C_{H}1-C_{H}2; (V) VH-C_{H}1-C_{H}2-C_{H}3; (vi) VH-C_{H}2-C_{H}3; (vii) VH-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}.

In any configuration of variable and constant domains, including any of the exemplary configurations listed above, the variable and constant domains may be either directly linked to one another or may be linked by a full or partial hinge or linker region. A hinge or linker region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric VH or V_{L} domain (e.g., by disulfide bond(s)).

As with full antibody molecules, antigen-binding fragments may be mono-specific or multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present invention using routine techniques available in the art.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia deflation, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest.” National institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In other embodiments, the integrin agonist is not an antibody. For example, the integrin agonist is a talin or the integrin agonist can be a kindlin. The integrin agonist can also be a talin is used in combination with a kindlin. In another example, the integrin agonist can be a reducing agent such as dithiothreitol or a lipid, such as oxysterol 25-hydroxycholesterol. In some embodiments, more than one integrin agonist is used, including any combination of integrin agonists mentioned herein. In some embodiments, the integrin agonist results in at least 50%, 60%, 70%, 80%, 90% or 100% cell growth in a culture method as claimed, when compared to the same method performed with the humanised TS2/16 antibody of the examples (e.g. as tested in the method provided in Example 4). In some embodiments, the integrin agonist results in at least 50%, 60%, 70%, 80%, 90% or 100% cell growth in a culture method as claimed, when compared to the same method performed with the mouse HUTS-4 antibody of the samples (e.g. as tested in the method provided in Example 6). In some embodiments, the methods of the invention result in an at least 10%, 20%, 50% increase in epithelial stem cell growth over 4 days relative to the same method performed without the integrin agonist. In some embodiments, the methods of the invention result in an at least 2-fold, 3-fold, 4-fold or 5-fold increase in epithelial stem cell growth over 4 days relative to the same method performed without the integrin agonist. Epithelial stem cell growth may be defined as the number of organoids and can be tested in accordance with the methods in Examples 3, 4 or 5.

In some embodiments, the methods of the invention result in the growth of a new organoid. In some embodiments, the methods of the invention result in the expansion of existing organoids.

The integrin agonist may be contacted with the epithelial stem cell or organoid comprising epithelial stem cells as part of the culture medium, as a component of an extracellular matrix or synthetic matrix, or as a separate component that is added to the culture vessel.

### Extracellular Matrix and Synthetic Matrix

Epithelial stem cells are normally grown in culture with an exogenous extracellular matrix that is known to support cell growth (e.g. see [20] which describes how cells are plated in Basement Membrane Extract (BME; Amsbio) or Matrigel (BD Biosciences)). Basement Membrane Extract (Amsbio) and Matrigel™ (BD Biosciences) are examples of commercially available extracellular matrices derived from basement membrane preparations from Engelbreth-Holm-Swarm (EHS) mouse sarcoma cells. However, the exact components of these extracellular matrices are not well-defined, which can produce an undesirable source of variability when culturing epithelial stem cells [14].

The component parts of extracellular matrix and the mechanism by which they act are not fully established. Synthetic replacements for the extracellular matrix, such as polymers and hydrogels, have been unable to faithfully replicate the growth efficiency achieved by extracellular matrices in epithelial stem cell culture. The use of extracellular matrix proteins, such as laminin and fibronectin instead of an extracellular matrix has also failed to replicate the growth efficiency achieved by extracellular matrices in epithelial stem cell culture.

Unexpectedly, the examples demonstrate that a culture medium comprising an integrin agonist can improve the growth of epithelial stem cells in a culture medium both in the presence and the absence of an extracellular matrix. Without wishing to be bound by any particular theory, a functional extracellular matrix involves a structural and a signalling component. Synthetic matrices alone are thought to provide the structural component only. The integrin agonist is therefore proposed to act as the long sought after missing signalling component and may provide additional structural support too.

The examples demonstrate that a culture medium comprising an integrin agonist can surprisingly improve epithelial stem cells growth in culture without an exogenous extracellular matrix. Therefore, in some embodiments the culture method does not involve contacting the cells with an exogenous extracellular matrix. For example, the culture method does not involve contacting the cells with an exogenous extracellular matrix comprising a glycoprotein, for example the culture method does not involve contacting the cells with a Basement Membrane Extract or Matrigel.

The integrin agonist is particularly useful for use with a synthetic matrix. Therefore, in some embodiments, the culture methods of the invention can comprise culturing epithelial stem cells in contact with a synthetic matrix. The components of a synthetic matrix can be controlled, which reduces the possible variability when culturing epithelial stem cells, which can help to improve the growth rate, expansion and/or the differentiation of the epithelial stem cells in culture. The synthetic matrix may comprise any polymer, e.g. a polyester, polyethylene glycol or a hydrogel. In some embodiments, the synthetic matrix comprises polyethylene glycol and/or hydrogel. In some embodiments, the synthetic matrix comprises a cross-linked polyethylene glycol (PEG) hydrogel.

The synthetic matrix may be provided in any suitable form including on a surface, a bead or as a coating (e.g. on a culture plate, culture vessel or bead). The bead is preferably a microbead. In the examples, an Ultra-Low Attachment Surface (Sigma Aldrich) is used. This surface is a covalently bound hydrogel layer that is hydrophilic and neutrally charged. Therefore, in some embodiments, the synthetic matrix is a hydrogel layer.

In some embodiments, a synthetic matrix comprises a biomaterial, preferably an extracellular matrix component. For example the biomaterial may comprise one or more glycoproteins (optionally selected from collagen, laminin, parlecan, fibronectin or an RGD attachment ligand of fibronectin) and/or one or more carbohydrates (optionally hyaluronic acid). One example of synthetic matrix which can be used is Pronectin (e.g. Sigma Z378666). Pronectin comprises a non-animal source polymer that incorporates multiple copies of the RGD attachment ligand of human fibronectin interspaced between repeated structural peptide units. It can be provided in the form of pronectin-F coated beads. The core bead is a solid copolymer sized 125-212 microns in diameter. Therefore, in some embodiments, the synthetic matrix comprises an RGD attachment ligand of fibronectin. In some embodiments, PEG gels can be enriched with extracellular matrix components such as fibronectin, laminin-111, collagen IV, hyaluronic acid and perlecan. In some embodiments, the fibronectin protein in the PEG gel can be replaced with a RGD (Arg—Gly—Asp) peptide [15].

Another example of a synthetic matrix is a synthetic hydrogel based on a four-armed, maleimide-terminated poly(ethylene glycol) macromer as described in [16]. The macromers are functionalised with adhesive peptides and crosslinked in the presence of cells to generate PEG-4MAL hydrogels. The hydrogel polymer density can be between 3.5-6.0% wt/vol, preferably the hydrogel polymer density is 4%. The hydrogel polymer can be formulated to include an RGD adhesive peptide or a GPQ-W crosslinking peptide.

The hydrogel polymer can be formulated with protein A/G. Protein AG is a recombinant fusion protein that combines IgG binding domains of both Protein A and Protein G. The protein A/G is connected to the hydrogel by a linker and provides a framework to which antibodies can be attached. The hydrogel can then be saturated with anti-integrin antibodies, which leads to the antibodies being presented in multivalent fashion. This formulation of the synthetic ECM helps to recreate the natural ECM environment found in a cell as both the structural and signalling components of the ECM are provided together. The hydrogel gel can be produced from dextran polymers or benzene-1,3,5-tricarboxamide polymers. In preferred embodiments, the hydrogel is made of Elastin Like Proteins (ELP) that have an antibody binding unit at their N-terminus (Z33 peptide).

The examples also demonstrate that the integrin agonist results in improved growth rates of epithelial stem cells in culture even when an exogenous extracellular matrix is present. Therefore, in some embodiments, the methods of the invention can comprise culturing epithelial stem cells in contact with an extracellular matrix (ECM). ECM is secreted by epithelial cells, endothelial cells, parietal endoderm like cells (e.g. Englebreth Holm Swarm Parietal Endoderm Like cells described in Hayashi at al. (2004) Matrix Biology 23:47 62) and connective tissue cells. It can be prepared by culturing ECM-secreting calls and isolating the ECM, or it is available commercially, e.g. as Matrigel or BME. It comprises of a variety of polysaccharides, water, elastin, and glycoproteins, wherein the glycoproteins comprise collagen, entactin (nidogen), fibronectin, and laminin. ECM as referred to herein is naturally produced by ECM-secreting cells. In some embodiments, the ECM is a basement membrane preparation derived from Englebreth Holm Swarm Parietal Endoderm Like cells (as described in Hayashi et al. (2004) Matrix Biology 23: 47 62).

The ECM when used in the context of the invention is an exogenous ECM (meaning that it is in addition to any extracellular matrix proteins that are naturally secreted by the epithelial stem cell when in contact with the culture medium of the invention, in other words it is produced by cells other than the epithelial stem cells being cultured in the method of the invention).

In some embodiments, the ECM is a three-dimensional matrix. In some embodiment, the cells are embedded in the ECM. In some embodiments, the cells are attached to on ECM. A culture medium of the invention may be diffused into a three-dimensional ECM. In other embodiments, the ECM is in suspension, i.e. the cells are in contact with the ECM in a suspension system. In some embodiments, the ECM is in the suspension at a concentration of at least 1%, at least 2% or at least 3%. In some embodiments, the ECM is in the suspension at a concentration of from 1% to about 10% or from 1% to about 5%. The suspension method may have advantages for upscale methods.

In some embodiments, the culture methods of the invention comprise culturing epithelial stem cells in contact with an extracellular matrix and/or a synthetic matrix. “In contact” means a physical or mechanical or chemical contact, which means that for separating said resulting organoid or population of epithelial cells from said matrix a force needs to be used. The culture medium and/or cells may be placed on, embedded in or mixed with the extracellular matrix or synthetic matrix.

In some embodiments, the culture medium is placed on top of the extracellular matrix or synthetic matrix. The culture medium can then be removed and replenished as and when required. In some embodiments, the culture medium is replenished every 1, 2, 3, 4, 5, 6 or 7 days. If components are “added” or “removed” from the media, then this can in some embodiments mean that the media itself is removed from the extracellular matrix or synthetic matrix and then a new media containing the “added” component or with the “removed” component excluded is placed on the extracellular matrix or synthetic matrix.

A three-dimensional matrix supports culturing of three-dimensional epithelial organoids. Therefore in some embodiments, the extracellular matrix or the synthetic matrix is a three-dimensional matrix.

The extracellular matrix or a synthetic matrix can further comprise an integrin agonist as described above.

### Culturing the Epithelial Stem Cell or Organoid

As the epithelial stem cells expand and/or differentiate to generate organoids, they are typically passaged (i.e. split), at regular intervals in accordance with methods known in the art. Passaging typically involves mechanically dissociating the organoids, optionally removing them from the extracellular matrix or synthetic matrix, collecting, washing, and plating at suitable ratios (e.g. 1:5 to 1:20) to allow efficient outgrowth or new organoids. The culture medium is also typically replenished at regular intervals, as required.

Therefore, in some embodiments the method further comprises passaging the cells twice per week, once per week, once every 10 days, once every two weeks, once every 5-20 days, preferably once every 7-14 days.

In some embodiments, the method further comprises plating cells at ratios between 1:5 and 1:20.

In some embodiments, the method further comprises replenishing the culture medium every 1-3 days, every 1-2 days, every second day, or every day. In a preferred embodiment, the method further comprises replenishing the culture medium every 1-3 days, every 1-2 days.

In some embodiments the method comprises culturing the epithelial stem cells for at least 5 passages, for example, at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 50, at least 60 passages or for between 6-40 passages, for example about 6-35 passages, 10-30 passages, or 12-25 passages. In some embodiments the method comprises culturing the epithelial stem cells for 8-50, 10-60, 15-50, 20-50; or 20-40 passages. In some embodiments, the method comprises culturing the epithelial stem cells for at least 2 weeks, at least 1 month, at least 2 months, more preferably at least 3, at least 4, at least 5, at least 6, at least 7, at least 8, at least 9, at least 10, at least 15, at least 20, at least 24, at least 26, at least 30 or more months, for example 3 or more years.

### Epithelial Stem Cells and Their Preparation for Culture

The epithelial stem cell can be a single cell or be part or a population of epithelial stem cells. The epithelial stem cell can be comprised in an organoid and/or an epithelial tissue explant. In certain embodiments, the epithelial stem cell is a mammalian epithelial stem cell, optionally a human or mouse epithelial stem cell. In preferred embodiments, the epithelial stem cell is a human epithelial stem cell. In some embodiments the epithelial stem cell is characterised by Lgr5 expression.

The epithelial stem cells are obtained from adult tissue, i.e. the epithelial stem cells are adult epithelial stem cells. In this context “adult” means mature tissue, i.e. includes newly-born baby or child but excludes embryonic or foetal. Alternatively, the epithelial stem cells are not derived from embryonic stem cells or embryonic stem cell lines, e.g. which have been differentiated in vitro.

The epithelial stem cell may be derived from colorectal, small intestine, stomach, pancreas, liver, lung, breast, prostate, kidney, mouth, nasopharynx, throat, hypopharynx, larynx, trachea, skin, fallopian tube, ovary, salivary gland, esophagus, hair follicle and/or cochlear tissue. In some embodiments the epithelial stem cells are colorectal cells. Methods for culturing epithelial stem cells from a variety of tissues have previously been described (e.g. in WO2009/022907, WO2010/090513, WO2012/014076, WO2012/168930, WO2015/173425, WO2016/083613, and WO2016/083612 and Clevers, Cell 165(7) 1586-1597, (2016)).

Cells taken directly from tissue, i.e. freshly isolated cells, are also referred to as primary cells. In some embodiments the epithelial stem cells are primary epithelial stem cells.

Primary cell cultures can be passaged to form secondary cell cultures. With the exception cancer cells, traditional secondary cell cultures have limited lifespan. After a certain number of population doublings (e.g. 50-100 generations) cells undergo the process of senescence and stop dividing. Cells from secondary cultures can become immortalized to become continuous cell lines. Immortalization can occur spontaneously, or may be virally- or chemically-induced. Immortalized cell lines are also known as transformed cells. By contrast, the methods of the present invention allow continuous passaging of epithelial stem cells through organoid growth without immortalisation or transformation. Thus in some embodiments, the epithelial stem cells are not immortalised or transformed cells or are not derived from an immortalised cell line or a transformed cell line. An advantage of the present invention is that the epithelial stem cells, undergoing multiple rounds of expansion and passage retain the characteristics of primary cells and have minimal or no genotypic or phenotypic changes. The starting population of epithelial stem cell(s) in the method of the invention may therefore be obtained or derived from an existing organoid and may be further cultured and expanded to generate new cells and organoids. Thus in some embodiments, the epithelial stem cell of population of epithelial stem cells is part of an organoid or isolated from an organoid, or wherein the population of epithelial stem cells is on organoid, part of an organoid or isolated from an organoid.

In some embodiments the epithelial stem cells are normal cells. In alternative embodiments, the epithelial stem cells are cancer stem cells. Accordingly, the cells may be obtained from a tumour, if required.

In some embodiments, the method comprises culturing a fragment of tissue which comprises epithelium. In some embodiments, the epithelial stem calls are isolated from a tissue fragment.

An organoid is preferably obtained using an epithelial cell from an adult tissue, optionally an epithelial stem cell from an adult tissue expressing Lgr5.

In some embodiments, an organoid originates from a single cell, optionally expressing Lgr5. Advantageously, this allows a homogenous population of cells to form. A single cell suspension comprising the epithelial stem cells can be mechanically generated. In some embodiments, the single cell suspension comprising the epithelial stem cells is generated using mechanical processes and/or enzymatic digestion. Mechanical processes include, but are not limited to dissection, micro-dissection and filtering.

In some embodiments, the starting culture is a clump or population of cells, for example, a population of cells contained in a colorectal fragment. Thus, the methods of the invention are not restricted to using single cells as the starting point.

The epithelial stem calls may be obtained by any suitable isolation method known in the art. In some embodiments, the epithelial layer is micro-dissected from a tissue sample, such as a surgical specimen or biopsy, digested by an enzyme, filtered and the resultant cell suspension is plated. In some embodiments, the micro-dissection is to remove other tissue types such as fat and muscle. In some embodiments, the enzyme is trypsin, collagenase or accutase. In some embodiments, the enzyme is trypsin, optionally at 0.126% trypsin. In some embodiments, the sample is incubated at 37° C., optionally the sample is then disrupted, for example with a pipette at repeated time intervals, such as every 2, 5, 10, or 15 minutes. In some embodiments, the sample is incubated in 0.125% trypsin at 37° C. and the sample is sheared using a pipette about every 10 minutes. In some embodiments, enzymatic digestion is performed for about 10 minutes, about 20 minutes, about 30 minutes, about 40 minutes, about 50 minutes, or about 60 minutes. In some embodiments, enzymatic digestion is completed by dilution in an appropriate medium. In some embodiments, the filtration step is performed using a 100 μm filter. In some embodiments, the resultant cell suspension from the mechanical process or enzymatic digestion is contacted with ECM and culture medium.

One method for obtaining the epithelial stem cells for culturing is based on the fact that epithelial stem cells express Lgr5 and/or Lgr6 on their surface; these proteins belong to the large G protein-coupled receptor (GPCR) superfamily (see, for example, WO 2009/022907, the contents of which are incorporated herein in their entirety). The Lgr subfamily is unique in carrying a large leucine rich ectodomain important for ligand binding. A preferred method therefore comprises preparing a cell suspension from said epithelial tissue as described above, contacting said cell suspension with an Lgr5 and/or 6 binding compound (such as an antibody, e.g. an anti-Lgr5 monoclonal antibody, e.g. as described in WO 2009/022907), isolating the Lgr5 and/or 6 binding compound, and isolating the stem cells from said binding compound.

Following culturing, the method may further comprise obtaining and/or isolating one or more epithelial stem cells or an organoid. For example, following culture of the stem cells, it may be useful to remove one or more stem cells and/or one or more organoids cultured in the culture medium from the culture medium for use in subsequent applications. For example, it may be useful to isolate a single cell for subsequent differentiation.

The organoids obtained and/or isolated in a method of the invention may recapitulate the characteristics of the starting epithelial stem cell. For example, cells cultured in an culture medium retain the same genotype and phenotype, and include cells with stem-like properties (e.g. cells characterised by Lgr5 expression). This means that cells taken from diseased tissue (e.g. from cancerous tissue), faithfully model the disease in question. Alternatively, cells cultured in a differentiation medium may result in an organoid comprising more mature, differentiated cell types (when compared to the starting cell).

### Cells Other Than Epithelial Stem Cells

The methods of the invention may be advantageous for all cell types and not only for epithelial stem cells and epithelial organoids. In particular, the integrin agonist may improve cell culture of many cell types. For example, the integrin agonist may be advantageous for other stem cells, particularly other stem cells that have been shown to benefit from culture with extracellular matrix (such as Matrigel or BME), including embryonic stem cells or induced pluripotent stem cells (e.g. see Clevers, Cell 165(7) 1586-1597, (2016)). Culture of embryonic stem cells and induced pluripotent stem cells often involves differentiation of the stem cells to more mature cell types. Therefore, the culture of embryonic stem cells and induced pluripotent stem cells with an integrin agonist may be particularly useful to the context of differentiation methods. The integrin agonist may also be particularly useful to hematopoietic stem cell culture—even though hematopoietic stem cells have been used routinely in many hospitals for many decades, it has not yet been possible to usefully expand these cells in culture. The addition of the integrin agonist is expected to improve hematopoietic stem cell culture.

Therefore, where the disclosure describes an embodiment concerning an epithelial stem cell, this embodiment is also disclosed for stem cells (in general), or for an embryonic stem cell or an pluripotent stem cell. Where the disclosure describes an embodiment concerning an epithelial stem cell, the same embodiment is also disclosed for a hematopoietic stem cell.

### Co-Culture

The method can further comprise co-culture with a non-epithelial cell type, optionally an immune cell. The method comprises the step of mixing the organoid as described herein with immuno cells in an in vitro culture. In a preferred embodiment, the organoid co-culture is maintained in a co-culture medium as described herein.

In some embodiments, the method for preparing the organoid-immune cell co-culture comprises one or more of the following steps: preparing the at least one organoid by culturing epithelial cells in an organoid culture medium; and/or culturing the immune cells in an immune cell expansion medium. In some embodiments, the method further comprises the step of obtaining the immune cells from an impure immune sample. Methods for isolating immune cells from impure immune samples are known in the art.

The invention also provides an organoid-immune cell co-culture obtained by the above method. The invention also provides uses of said organoid-immune cell co-culture in drug screening, toxicology screening, research and drug development.

### Culture Medium

Culture media suitable for epithelial stem cells are well known in the art, e.g. as described in WO2009/022907, WO2010/090513, WO2012/014076, WO2012/168930, WO2015/173425, WO2018/083613, WO2016/083612 and WO2017/149025. The culture media mentioned in these documents are incorporated herein by reference and any of these may be used in the context of the invention.

In some embodiments, the culture medium suitable for culturing epithelial stem cells can comprise one or more of a Wnt agonist a BMP inhibitor, a mitogenic growth factor and a TGF-beta inhibitor. For example, the culture medium suitable for epithelial stem cells comprises a Wnt agonist. The culture medium suitable for epithelial stem cells can further comprises a mitogenic growth factor and/or a BMP inhibitor.

In preferred embodiments the culture medium suitable for epithelial stem cells comprises a Wnt agonist, a BMP inhibitor, a mitogenic growth factor and a TGF-beta inhibitor. The culture medium suitable for epithelial stem cells can further comprise one or more of a p38 inhibitor, a cAMP agonist, a prostaglandin pathway activator, nicotinamide, gastrin, B27 and N-acetylcysteine.

In some embodiments the culture medium comprises a basal medium for human or animal cells (such as DMEM/F12 optionally including B27), an R-spondin family protein, a mitogenic growth factor (such as EGF), a BMP inhibitor (such as noggin), a TGF-beta inhibitor (such as A83-01), a p38 inhibitor (such as SB202190) and optionally nicotinamide and N-acetylcysteine.

In some embodiments the culture medium comprises advanced DMEM/F12 medium including B27, nicotinamide, N-acetylcysteine, noggin, R-spondin 1, EGF, WNT conditioned media (50%, produced using stably transfected L cells), TGF-b type I receptor inhibitor A83-01 and P38 inhibitor SB202190.

In some embodiments, the culture medium suitable for epithelial stem cells is a culture medium suitable for expanding epithelial stem cells. The skilled person will understand that the culture media mentioned above are particularly useful for expanding epithelial stem cells.

In other embodiments, the culture medium suitable for epithelial stem cells is a culture medium suitable for differentiating epithelial stem cells.

The invention provides a culture medium suitable for culturing epithelial stem cells as defined herein, wherein the culture medium further comprises an integrin agonist as defined herein.

### WNT Agonist

The culture medium suitable for culturing epithelial stem cells can comprise one or more Wnt agonist. A Wnt agonist is defined herein as an agent that activates or enhances TCF/LEF-mediated transcription in a cell.

The canonical Wnt signalling pathway is defined by a series of events that occur when the cell-surface Wnt receptor complex, comprising a Frizzled (FZD) receptor and LRP is activated, usually by an extracellular signaling molecule, such as a member of the Wnt family of secreted glycoproteins. This results in the activation of Dishevelled family proteins which inhibit a complex of proteins that includes axin, GSK-3, and the protein APC to degrade intracellular β-catenin. The resulting enriched nuclear β-catenin enhances transcription by TCF/LEF family transcription factors (Driehuis & Clevers, British Journal of Pharmacology (2017) 174 4547-4563).

The R-spondin/Rnf43/Lgr module further regulates canonical Wnt signalling. In the absence of R-spondin, E3 ligases RNF43/ZNRF3 ubiquinate FZD, thus marking it for degradation by the proteasome and inhibiting Wnt signalling. When extracellular R-spondin is present, it can interact with membrane-spanning E3 ligases RNF43/ZNRF3 via an Lgr receptor, preventing the action of the E3 ligases. Lgr receptors, including Lgr4, Lgr5 and Lgr6, but particularly Lgr5, are expressed on epithelial stem cells. R-spondin is recruited by these stem cell markers to enhance Wnt signalling, and thus R-spondin and Lgr interact to promote proliferation and retain the stem cell multipotency. For these reasons, R-spondin family proteins have been shown to be particularly useful in epithelial stem cell culture for obtaining long-lived organoid cultures.

The one or more Wnt agonist in the culture medium may therefore be selected from a Wnt ligand from the Wnt family of secreted glycoproteins, an inhibitor of intracellular β-catenin degradation, a GSK-3 inhibitor, activators of TCF/LEF, an inhibitor of RNF43 or ZNRF3, and R-spondin family proteins. In some embodiments, the Wnt agonist in the culture medium comprises an R-spondin family protein and a GSK-3 inhibitor, and optionally further comprises a Wnt ligand from the Wnt family of secreted glycoproteins.

The R-spondin family protein (also referred to herein as “R-spondin”) may be selected from R-spondin 1, R-spondin 2, R-spondin 3, R-spondin 4 and analogs, fragments, variants and derivatives thereof. In this context, the fragment, variant or derivative is capable of preventing the action of the E3 ligases RNF43/ZNRF3 on the Wnt receptor complex. R-spondin 1, R-spondin 2, R-spondin 3 and R-spondin 4 (also referred to herein as “R-spondin 1-4”) are all characterised by two amino-terminal furin-like repeats, which are necessary and sufficient for Wnt signal potentiation, and a thrombospondin domain situated more towards the carboxyl terminus members (Lau et al. Genome Biol. 2012;13(3):242 (2012). Examples of R-spondin fragments, variants and derivatives suitable for use in the invention are known to the skilled person (e.g. see Example 2 of WO 2012/140274, which describes furin domain fragments which are capable of enhancing Wnt signalling and which are incorporated herein by reference). Examples of R-spondin family protein analogs include, for example, antibodies that interact with RNF43/ZNRF3/Lgr. Agonistic anti-Lgr5 antibodies that can enhance Wnt signalling are known in the art (e.g. see antibody 1D9 described in Example 3 of WO 2012/140274).

Many GSK-3 inhibitors are known in the art (e.g. see Greengard, P., and Meijer, L. (2004) Structural basis for the synthesis of indirubins as potent and elective inhibitors of glycogen synthase kinase-3 and cyclin-dependent kinases. J Med Chem 47: 935-946; and Thomas Kramer, Boris Schmidt, and Fabio Lo Monte, “Small-Molecule Inhibitors of GSK-3: Structural Insights and Their Application to Alzheimer's Disease Models,” International Journal of Alzheimer's Disease, vol. 2012. Article ID 381029, 32 pages, 2012. https://doi.org/10.1155/2012/331029) and are available commercially (e.g. see the list available from Santa Cruz Biotechnology here: https://www.scbt.com/scbt/browse/GSK-3-beta-Inhibitors/_/N-x6oud). Any of these GSK-3 inhibitors are suitable for use in the context of the invention and the skilled person would be able to determine a suitable concentration using IC50 values.

CHIR-99021 (CAS: 252917-06-9; 6-[[2-[[4-(2,4-Dichlorophenyl)-5-(5-methyl-1H-imidazol-2-yl)-2-pyrimidinyl]amino]ethyl]amino]-3-pyridinecarbonitrile: CT99021) is a potent and selective inhibitor of GSK-3. Other aminopyrimidine inhibitors with an IC50 value of 0.6 nM to 7 nM include CHIR98014 (Axon, Cat 1126), CHIR98023, CHIR83021 (see above), TWS119 (Tocris, Cat 3835). Therefore, in some embodiments, the GSK-3 inhibitor is an aminopyrimidine inhibitor, optionally selected from CHIR98014, CHIR98023, CHIR99021 or TWS119. In some embodiments the GSK-3 inhibitor is CHIR-99021.

Any suitable concentration of a GSK-3 inhibitor (e.g. CHIR-90021) may be used, for example, between 10 nM and 500 μM, between 10 nM, and 400 μM, between 10 nM and 300 μM, between 10 nM and 200 μM, between 10 nM and 100 μM or between 20 nM and 50 μM, or wherein the final concentration is about 3 μM.

The Wnt ligand from the Wnt family of secreted glycoproteins may be selected from Wnt-I/Int-1, Wnt-2/Irp (InM-related Protein) Wnt-2b/13, Wnt-3/Int-4, Wnt-3a (R&D systems), Wnt-4, Wnt-5a, Wnt-5b, Wnt-6 (Kinoshi H et al 2001 Biochem Biophys Res Com 283 798-805), Wnt-7a (R&D systems). Wnt-7b, Wnt-8a/8d, Wnt-8b, Wnt-9a/14, Wnt-9b/14b/15, Wnt-10a, Wnt-10b/12, WnM I, and Writ-16. An overview of human Wnt proteins is provided in “THE WNT FAMILY OF SECRETED PROTEINS”, R&D Systems Catalog, 2004. In some embodiments, the Wnt ligand is Wnt-3a, Wnt-5 or Wnt-6a, or optionally is Wnt-3a. Addition of a soluble Wnt ligand has been shown to be particularly advantageous for expansion of human epithelial stem cells (e.g. as described in WO2012/168930).

The Wnt agonist is preferably added to the media in an amount effective to stimulate a Wnt activity in a cell by at least 10%, more preferred at least 20%, more preferred at least 30%, more preferred at least 50%, more preferred at least 70%, more preferred at least 90%, more preferred at least 100%, relative to a level of said Wnt activity in the absence of said molecule, as assessed in the same cell type. As is known to a skilled person, Wnt activity can be determined by measuring the transcriptional activity of Wnt, for example by pTOPFLASH and pTOPFLASH Tcf luciferase reporter constructs (Korinek et al., 1997. Science 275:1784-1787).

A soluble Wnt agonist, such as Wnt-3a, may be provided in the form of Wnt conditioned medium. For example, about 10% to about 30%, e.g. about 10 ng/ml to about 10 μg/ml, preferably about 1 μg/ml. Wnt conditioned medium may be used. In other embodiments, a surrogate Wnt agonist may be used (e.g. as described in [19] and in the examples).

R-spondin may be provided in the form of R-spondin conditioned medium or in the form of recombinant protein, for example, about 18 ng/ml to about 500 ng/ml, about 10 ng/ml to about 400 ng/ml, about 10 ng/ml to about 300 ng/ml, about 10 ng/ml to about 250 ng/ml, about 60 ng/ml to about 250 ng/ml, about 100 ng/ml to about 250 ng/ml, or about 150 ng/ml to about 250 ng/ml, preferably about 150 ng/ml to about 250 ng/ml, R-spondin may be used. For example, the final concentration of R-spondin in the culture medium may be about 10 ng/ml, about 25 ng/ml, about 50 ng/ml, about 75 ng/ml, about 100 ng/ml, about 125 ng/ml, about 150 ng/ml, about 175 ng/ml, about 200 ng/ml, about 225 ng/ml, about 250 ng/ml, about 275 ng/ml, about 300 ng/ml, about 325 ng/ml, about 350 ng/ml, about 375 ng/ml, about 400 ng/ml, about 450 ng/ml, or about 500 ng/ml.

One or more, for example 2, 3, 4 or more Wnt agonists may be used in the culture medium.

### Mitogenic Growth Factors

The culture medium suitable for culturing epithelial stem cells can comprises a mitogenic growth factor. Mitogenic growth factors typically induce cell division via the mitogen-activated protein kinase signalling pathway. Many receptor tyrosine kinase ligands are mitogenic growth factors in some embodiments, the mitogenic growth factor can bind to a receptor tyrosine kinase. In some embodiments, the mitogenic growth factor can bind to more than one receptor tyrosine kinase. In some embodiments, the one or more mitogenic growth factor binds to a receptor tyrosine kinase such as EGFR, an FGFR or HGFR, optionally wherein the one or more mitogenic growth factors are selected from EGF, FGF and HGF.

Epidermal growth factor receptor (EGFR), also known as ErbB1 or HER1, is a cell surface receptor for members of the epidermal growth factor (EGF) family of extracellular protein ligands. EGFR is a receptor tyrosine kinase and belongs to the HER family of receptors which comprise four related proteins (EGFR (HER1/ErbB1), ErbB2 (HER2), ErbB3 (HER3) and ErbB4 (HER4)). The HER receptors are known to be activated by binding to different ligands, including EGF, TGF-alpha, heparin-binding EGF like growth factor, neuregulin, amphiregulin, betacellulin, and epiregulin. After a ligand binds to the extracellular domain of the receptor, the receptor forms functionally active dimers (EGFR-EGFR (homodimer) or EGFR-HER2, EBFR-HER3, EGFR-HER4 (heterodimer)). Dimerization induces the activation of the tyrosine kinase domain, which leads to autophosphorylation of the receptor on multiple tyrosine residues. This leads to recruitment of a range of adaptor proteins (such as SHC, GRB2) and activates a series of intracellular signalling cascades to affect gene transcription. Therefore, in some embodiments, the mitogenic growth factor binds to EGFR, HER1 HER2, HER3 or HER4. In some embodiments, the mitogenic growth factor binds to EGFR. In some embodiments, a HER2-4 ligand is included in the culture medium in addition to an EGFR ligand. For example, in some embodiments, neuregulin is included in the culture medium in addition to EGF. Neuregulin has been shown to be advantageous for culture of lung and breast tissue (e.g. see WO2016/083813, and WO2016/083612). In some embodiments, the one or more mitogenic growth factor in the culture medium is EGF. Any suitable EGF may be used, for example, EGF obtained from Peprotech. EGF is preferably added to the basal culture medium at a final concentration of between 0.1 ng/ml and 500 ng/ml, between 0.1 ng/ml and 400 ng/ml, between 0.1 ng/ml and 300 ng/ml, between 0.1 ng/ml and 200 ng/ml, between 0.1 ng/ml and 100 ng/ml, between 1 ng/ml and 100 ng/ml, or wherein the final concentration of a mitogenic growth factor is approximately 1 ng/ml, 2 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 250 ng/ml or 500 ng/ml and not higher than 500 ng/ml. A more preferred concentration is at least 50 and not higher than 100 ng/ml. An even more preferred concentration is about 50 ng/ml. FGFs stimulate cells by interacting with cell surface tyrosine kinase receptors (FGFR). Four closely related receptors (FGFR1-FGFR4) have been identified. Therefore, in some embodiments, the mitogenic growth factor binds to an FGF receptor family member. FGF receptor family members include (but are not limited to) FGFR1, FGFR2, FGFR3 or FGFR4. FGFR1-FGFR3 genes have been shown to encode multiple isoforms, and these isoforms can be critical in determining ligand specificity. There are several FGFs that bind to the FGF receptor family members, including (but not limited to) FGF2, FGF4, FGF7 and FGF 10. These are commercially available. Therefore, in some embodiments, the mitogenic growth factor is an FGF. In some embodiments, the FGF is selected from FGF2, FGF4, FGF7 and FGF10. In preferred embodiments, the FGF is FGF2 and/or FGF10. In a most preferred embodiment, the FGF is FGF2 and FGF10.

Most FGFs bind more than one receptor (Ornitz J Biol Chem 1988 Feb. 27:273 (9) 5349-57). However. FGF10 and FGF7 are unique among FGFs in that they interact only with a specific isoform of FGFR2, designated FGFR2b which is expressed exclusively by epithelial cells (Igarashi, J Biol Chem. 1998 273(21):13230-5). Therefore, in some embodiments the mitogenic growth factor binds to FGFR2b, FGF10 has been shown to be particularly useful in the culture medium. FGF10 is able to bind to FGFR2 or FGFP4. Therefore, in some embodiments, the mitogenic growth factor binds to FGFR2 or FGFR4. FGF2 binds to all of FGFR1, FGFR2, FGFR3 and FGFR4. Therefore, in some embodiments, the mitogenic growth factor binds to all of FGFR1, FGFR2, FGFR3 and FGFR4.

In some embodiments, the final concentration of FGF is between 0.1 ng/ml and 500 ng/ml, between 0.1 ng/ml and 400 ng/ml between 0.1 ng/ml and 300 ng/ml, between 0.1 ng/ml and 200 ng/ml, between 0.1 ng/ml and 100 ng/ml, between 1 ng/ml and 100 ng/ml, or wherein the final concentration of a mitogenic growth factor is about 1 ng/ml, 2 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 250 ng/ml or 500 ng/ml.

In some embodiments, the one or more mitogenic growth factor in the culture medium is FGF10. Preferred concentrations for FGF10 are approximately between 0.1 ng/ml and 500 ng/ml, between 0.1 ng/ml and 400 ng/ml, between 0.1 ng/ml and 300 ng/ml, between 0.1 ng/ml and 200 ng/ml, between 0.1 ng/ml and 100 ng/ml, between 1 ng/ml and 100 ng/ml, or wherein the final concentration of a mitogenic growth factor is approximately 1 ng/ml, 2 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 250 ng/ml or 500 ng/ml and not higher than 500 ng/ml. A more preferred concentration for FGF10 is about 10 ng/ml.

In some embodiments, the one or more mitogenic growth factor in the culture medium is FGF2. Preferred concentrations for FGF2 are approximately between 0.1 ng/ml and 500 ng/ml, between 0.1 ng/ml and 400 ng/ml, between 0.1 ng/ml and 300 ng/ml, between 0.1 ng/ml and 200 ng/ml, between 0.1 ng/ml and 100 ng/ml, between 1 ng/ml and 100 ng/ml, or wherein the final concentration of a mitogenic growth factor is approximately 1 ng/ml, 2 ng/ml, 5 ng/ml, 10 ng/ml, 20 ng/ml, 30 ng/ml, 40 ng/ml, 50 ng/ml, 80 ng/ml, 70 ng/ml, 80 ng/ml, 90 ng/ml, 100 ng/ml, 250 ng/ml or 500 ng/ml and not higher than 500 ng/ml. A more preferred concentration for FGF2 is about 5 ng/ml.

Hepatocyte growth factor/scatter factor (HGF/SF) is a morphogenic factor that regulates cell growth, cell motility, and morphogenesis by activating a tyrosine kinase signalling cascade after binding to the proto-oncogenic HGFR. The HGFR is also known as the c-Met receptor HGF has been shown to be useful. In epithelial stem cell culture. Therefore, in some embodiments the mitogenic growth factor binds HGFR. In some embodiments, the mitogenic growth factor is HGF. Any suitable HGF may be used for example, HGF obtained from Peprotech. Preferred concentrations for HGF are about 1, 10, 20, 25, 50 ng/ml, not higher than 50 ng/ml.

In some embodiments, more then one mitogenic growth factor is included in the culture medium, e.g. two or three mitogenic growth factors, for example. In some embodiments, the one or more mitogenic growth factors in the culture medium are EGF and FGF. In some embodiments, the one or more mitogenic growth factors in the culture medium are EGF, FGF2 and FGF10. In some embodiments, the one or more mitogenic growth factors in the culture medium are EGF, optionally at a final concentration of about 50 ng/ml, FGF2, optionally at a final concentration of about 5 ng/ml and FGF10, optionally at a final concentration of about 10 ng/ml.

In some embodiments hepatocyte growth factor (HGF) is also present in the presence or absence of EGF and/or FGF.

In some embodiments, the final concentration of each mitogenic growth factor is between 0.1 ng/ml and 500 ng/ml, between 0.1 ng/ml and 400 ng/ml, between 0.1 ng/ml and 300 ng/ml, between 0.1 ng/ml and 200 ng/ml, between 0.1 ng/ml and 100 ng/ml, between 1 ng/ml and 100 ng/ml, or wherein the final concentration of a mitogenic growth factor is about 1 ng/ml, 2 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 250 ng/ml or 500 ng/ml.

### BHP Inhibitor

The culture medium suitable for culturing epithelial stem cells can comprises a BMP inhibitor. BMPs bind as a dimeric ligand to a receptor complex consisting of two different receptor serine/threonine kinases, type I and type II receptors. The type II receptor phosphorylates the type I receptor, resulting in the activation of this receptor kinase. The type I receptor subsequently phosphorylates specific receptor substrates (SMAD), resulting in a signal transduction pathway leading to transcriptional activity.

A BMP inhibitor is defined as an agent that binds to a BMP molecule to form a complex wherein the BMP activity is neutralized, for example by preventing or inhibiting the binding of the BMP molecule to a BMP receptor. Alternatively, said inhibitor is an agent that acts as an antagonist or reverse agonist. This type of inhibitor binds with a BMP receptor and prevents binding of a BMP to said receptor. An example of a latter agent is an antibody that binds a BMP receptor and prevents binding of BMP to the antibody-bound receptor.

A BMP inhibitor may be added to the media in an amount effective to inhibit a BMP-dependent activity in a cell to at most 90%, more preferred at most 80%, more preferred at most 70%, more preferred at most 60%, more preferred at most 30%, more preferred at most 10%, more preferred 0%, relative to a level of a BMP activity in the absence of said inhibitor, as assessed in the same cell type. As is known to a skilled person, a BMP activity can be determined by measuring the transcriptional activity of BMP, for example as exemplified in Zilbertberg at al. 2007 BMC Cell Biol. 8:41.

Several classes of natural BMP-binding proteins are known, including noggin (Peprotech), Chordin and chordin-like proteins (R&D systems) comprising chordin domains, Follistatin and follistatin-related proteins (R&D systems) comprising a follistatin domain, DAN and DAN-like proteins (R&D systems) comprising a DAN cysteine-knot domain, sclerostin/SOST (R&D systems), decorin (R&D systems), and alpha-2 macroglobulin (R&D systems).

Therefore, in some embodiments, the BMP inhibitor is selected from noggin, DAN and DAN-like proteins including Cerberus and Gremlin (R&D systems). These diffusible proteins are able to bind a BMP ligand with varying degrees of affinity and inhibit their access to signalling receptors. The addition of any of these BMP inhibitors to the basal culture medium prevents the loss of stem cells. A preferred BMP inhibitor is noggin.

In some embodiments, the final concentration of the BMP inhibitor (e.g. noggin) is about 10 ng/ml to about 500 ng/ml, about 10 ng/ml to about 400 ng/ml, about 10 ng/ml to about 300 ng/ml, about 10 ng/ml to about 250 ng/ml, about 50 ng/ml to about 250 ng/ml, about 50 ng/ml to about 150 ng/ml, or wherein the final concentration is about 100 ng/ml.

### TGF-Beta Inhibitor

The culture medium suitable for culturing epithelial stem cells can comprises a TGF beta inhibitor. The presence of a TGF-beta inhibitor in the expansion media is particularly advantageous for increasing human organoid formation efficiency. TGF-beta signalling typically begins with binding of a TGF-beta superfamily ligand to a type II receptor which recruits and phosphorylates a type I receptor. The type I receptor then phosphorylates SMADs, which act as transcription factors in the nucleus and regulate target gene expression.

The TGF-beta superfamily ligands comprise bone morphogenic proteins (BMPs), growth and differentiation factors (GDPs), anti-müllerian hormone (AMH), activin, nodal and TGF-betas. In general, Smad2 and Smad3 are phosphorylated by the ALK4, 5 and 7 receptors in the TGF-beta/activin pathway. By contrast, Smad1, Smad5 and Smad8 are phosphorylated as part of the bone morphogenetic protein (BMP) pathway. However, in the context of this invention, this skilled person will understand that a “TGF-beta inhibitor” or an “inhibitor of TGF-beta signalling” is an inhibitor of the TGF-beta pathway which involves ALK4, 5 and 7, and which involves Smad2 and Smad3. The TGF beta inhibitor is not a BMP inhibitor, i.e. the skilled person would understand that noggin is not a TGF-beta inhibitor in the context of this disclosure. In some embodiments, a BMP inhibitor is added to the culture medium in addition to the TGF-beta inhibitor (see below).

Thus the TGF-beta inhibitor is any agent that reduces the activity of the IGF-beta signalling pathway, also referred to herein as the ALK4, ALK5 or ALK7 signalling pathway. There are many ways of disrupting the TGF-beta signalling pathway that are known in the art and that can be used in conjunction with this invention. For example, the TGF-beta signalling may be disrupted by: inhibition of TGF-beta expression by a small-interfering RNA strategy; inhibition of furin (a TGF-beta activating protease); inhibition of the pathway by physiological inhibitors; neutralisation of TGF-beta with a monoclonal antibody; inhibition with small-molecule inhibitors of TGF-beta receptor kinase 1 (also known as activin receptor-like kinase, ALK5), ALK4, ALK7; inhibition of Smad 2 and Smad 3 signalling, e.g. by overexpression of their physiological inhibitor, Smad 7, or by using thioredoxin as an Smad anchor disabling Smad from activation (Fuchs, O. Inhibition of TGF-Signaling for the Treatment of Tumor Metastasis and Fibrotic Diseases. Current Signal Transduction Therapy, Volume 6, Number 1, January 2011, pp. 29-43(15)).

Various methods for determining if a substance is a TGF-beta inhibitor are known and might be used in conjunction with the invention. For example, a cellular essay may be used in which cells are stably transfected with a reporter construct comprising the human PAI-1 promoter or Smad binding sites, driving a luciferase reporter gene. Inhibition of luciferase activity relative to control groups can be used as a measure of compound activity (De Gouville et al., Br J Pharmacol 2005 May; 145(2): 166-177).

A TGF-beta inhibitor according to the present invention may be a protein, peptide, small-molecule, small-interfering RNA, antisense oligonucleotide, aptamer or antibody. The inhibitor may be naturally occurring or synthetic.

In some embodiments the TGF-beta inhibitor is a small molecule inhibitor such as A83-01. A83-01 is a commercially available selective inhibitor of ALK4, ALK5 and ALK7 (Tocris cat. no. 2939). It is described in the catalog as a potent inhibitor of TGF-β type I receptor ALK5 kinase, type I activin/nodal receptor ALK4 and type I nodal receptor ALK7 (IC₅₀ values are 12, 45 and 7.5 nM respectively), which blocks phosphorylation of Smad2, and which only weakly inhibits ALK-1, -2, -3, -6 and MAPK activity. Other commercially available inhibitors with similar properties include, but are not limited to A77-01, LY2157299, LV2109761, LY3200882, QW788388, Pirfenidone, RepSox, SB431542, SB505124, SB525334, LY364947, SD-208 and Vactosertib. The IC50 values for these inhibitors are known in the art and the skilled person would be able to select a suitable inhibitor at suitable concentration based on the teaching provided in the examples of this application.

Therefore, in some embodiments, the TGF-beta inhibitor is an inhibitor of ALK4, ALK5 and ALK7, optionally a selective inhibitor of ALK4, ALK5 or ALK7. For example, the TGF-beta inhibitor may bind to and directly inhibit ALK4, ALK5 and/or ALK7. In some embodiments, the TGF-beta inhibitor is an inhibitor that blocks phosphorylation of Smad2. In some embodiments, the TGF-beta inhibitor is selected from A83-01, A77-01 (Tocris cat. no. 6712) LY2157299 (Selleckchem cat. no. S2230), LY2109761 (Selleckchem cat. no. S2704), LY3200882 (Selleckchem cat. no. S8772), GW788388 (Tocris cat no 3264), Pirfenidone, RepSox (Tocris cat. no. 3742), SB431542 (Tocris cat. no. 1614), SB505124 (Tocris cat. no. 3263), SB525334 (Tocris cat. no. 3211), LY364947 (Tocris cat. no 2718), SD-208 (Tocris cat. no 3269), and Vactosertib (Selleckchem cat. no. S7530). In some embodiments, the TGF-beta inhibitor is A83-01.

In some embodiments, no more than one TGF beta inhibitor is present in the culture medium. In other embodiments, more than one TGF beta inhibitor is present in the culture medium, e.g. 2, 3, 4 or more.

In some embodiments, the final concentration of the TGF beta inhibitor is between 1 nM and 100 μM, between 10 nM and 100 μM, between 100 nM and 10 μM, or about 1 μM, for example, wherein the final concentration of the one or more inhibitor is between 10 nM and 100 μM, between 100 nM and 10 μM, or about 500 nM. In some embodiments, the final concentration of the TGF beta inhibitor is at least 6 nM, for example, at least 50 nM, at least 100 nM, at least 300 nM, at least 450 nM, at least 475 nM, for example 5 nM-500 mM, 10 nM-100 mM, 50 nM-700 μM, 50 nM-10 mM, 100 nM-1080 nM, 350-650 nM or more preferably about 500 mM. In some embodiments, the TGF-beta inhibitor is A83-01 at a final concentration of about 500 nM.

The skilled person would appreciate that the appropriate final concentration of a TGF-beta inhibitor is dependent on the TGF-beta inhibitor in question and the skilled person would know how to determine the concentration of other TGF beta inhibitors for use in the invention.

### Nicotinamide

In some embodiments, the culture medium suitable for epithelial stem cells further comprises nicotinamide. Nicotinamide is an amide derivative of vitamin B3, a poly (ADP-ribose) polymerase (PARP) inhibitor, and represents the primary precursor of NAD+. It is available commercially (e.g. from Stemcell Technologies Cat. 07154). In some embodiment, Nicotinamide is present at 7-15 mM, for example about 10 mM.

### Prostaglandin Pathway Activator

The activator of the prostaglandin signalling pathway (also called a prostaglandin pathway activator) may be any one or more of the compounds selected from the list comprising: phospholipids, arachidonic acid (AA), prostaglandin E2 (PGE2), prostaglandin G2 (PGG2), prostaglandin F2 (PGF2), prostaglandin H2 (PGH2), prostaglandin D2 (PGD2). In some embodiments, the activator of the prostaglandin signalling pathway is PGE2 and/or AA. In some embodiments, the activator of the prostaglandin signalling pathway is PGE2.

In some embodiments, the final concentration of the activator of the of the prostaglandin signalling pathway (e.g. PGE2) is between 10 nM and 500 μM, between 10 nM, and 400 μM, between 10 nM and 300 μM, between 10 nM and 200 μM, between 10 nM and 100 μM or between 20 nM and 50 μM, or wherein the final concentration is about 1 μM.

The skilled person would appreciate that the appropriate final concentration of the activator of the of the prostaglandin signalling pathway is dependent on the activator of the of the prostaglandin signalling pathway in question and the skilled person would know how to determine the concentration of other activators of the of the prostaglandin signalling pathway for use in the invention.

cAMP Activator
The cAMP pathway activator may be any suitable activator which increases the levels of cAMP in a cell. The cAMP pathway involves activation of many types of hormone and neurotransmitter G-protein cooked receptors. Binding of the hormone or neurotransmitter to its membrane-bound receptor induces a conformational change in the receptor that leads to activation of the α-subunit of the G-protein. The activated G subunit stimulates, while the non-activated G subunit inhibits adenylyl cyclase. Stimulation of adenylyl cyclase catalyses the conversion of cytoplasmic ATP to cAMP thus increasing the levels of cAMP in the cell.

Therefore, in some embodiments, the cAMP pathway activator is an adenylyl cyclase activator or a cAMP analog. Examples of suitable adenylyl cyclase activators include forskolin, a forskolin analog and cholera toxin. Examples of forskolin analogs are known in the art and include NKH477 (e.g. catalogue no. Tocris 1603). Examples of cAMP analogs are also known in the art, and include for example, 8-bromo-cAMP. 8-bromo-cAMP is a cell permeable cAMP analog having greater resistance to hydrolysis by phosphodiesterases than cAMP. In some embodiments, the cAMP pathway activator is therefore selected from forskolin, cholera toxin, NKH477 and 8-bromo-cAMP. In some embodiments, the cAMP pathway activator is forskolin. In some embodiments, the cAMP pathway activator is not cholera toxin.

cAMP pathway activators can be identified using methods known in the art, for example, using a competitive immunoassay which measures cAMP levels. The CatchPoint® Cyclic-AMP Fluorescent Assay Kit (Molecular Devices LLC) is an example of a commercially available kit for carrying out such an immunoassay. The cAMP in the sample or standard competes with horseradish peroxidase (HRP)-labeled cAMP conjugate for binding sites on the anti-cAMP antibodies. In the absence of cAMP, most of the HRP-cAMP conjugate is bound to the antibody. Increasing concentrations of cAMP competitively decrease the amount of bound conjugate, thus decreasing measured HRP activity. A cAMP pathway activator would result in increased levels of cAMP and decreased measured HRP activity, compared to a control.

In some embodiments, the final concentration of the cAMP pathway activator (e.g. forskolin) is between 10 nM and 500 μM, between 10 nM, and 400 μM, between 10 nM and 300 μM, between 10 nM and 200 μM, between 10 nM and 100 μM or between 20 nM and 50 μM, or wherein the final concentration is about 1 μM. In some embodiments the cAMP pathway activator is forskolin. In some embodiments the final concentration of forskolin is about 1 μM.

The concentration selected may depend upon the cAMP pathway activator used and can be determined by the person skilled in the art depending upon the potency of the cAMP pathway activator. For example, NKH477 is generally more potent then 8-bromo-cAMP and forskolin. A more potent cAMP pathway activator can be used at lower concentrations to the same effect.

### Additional Components

Basal media for cell culture typically contain a large number of ingredients, which are necessary to support maintenance of the cultured cells. Suitable combinations of ingredients can readily be formulated by the skilled person, taking into account the following disclosure. A basal medium for use in the invention will generally comprises a nutrient solution comprising standard cell culture ingredients, such as amino acids, vitamins, lipid supplements, inorganic salts, a carbon energy source, and a buffer, as described in more detail in the literature and below. In some embodiments, the culture medium is further supplemented with one or more standard cell culture ingredient, for example selected from amino adds, vitamins, lipid supplements, inorganic salts, a carbon energy source, and a buffer. Suitable basal media will be known to the skilled person and are available commercially, e.g. non-limiting examples include Dulbecco's Modified Eagle Media (DMEM). Advanced-DMEM, Minimal Essential Medium (MEM), Knockout-DMEM (KO-DMEM), Glasgow Minimal Essential Medium (GMEM), Basal Medium Eagle (BME), DMEM/Ham's F12, Advanced DMEM/Ham's F12, Iscove's Modified Dulbecco's Media and Minimal Essential Media (MEM), Ham's F-10, Ham's F-12, Medium 199, and RPMI 1640 Media. For example, the basal medium may be Advanced DMEM, preferably supplemented with glutamax, penicillin/streptomycin and HEPES.

The culture medium suitable for epithelial stem cells may be supplemented with one or more of the compounds selected from the group consisting of gastrin, B27, N-acetylcystein and N2. Thus in some embodiments the culture medium described above further comprises one or more components selected from the group consisting of: gastrin, B27, N2 and N-Acetylcysteine. B27 (Invitrogen), N-Acetylcysteine (Sigma) and N2 (Invitrogen), Gastrin (Sigma) are believed to control proliferation of the cells and assist with DNA stability. In some embodiments, the culture medium further comprises B27 and N-acetylcystein.

In some embodiments, the B27 supplement is ‘B27 Supplement minus Vitamin A’ (available from Invitrogen, Carlsbad Calif.; www.invitrogen.com, currently catalog no. 12587010; and from PAA Laboratories GmbH, Pasching, Austria; www.paa.com; catalog no. F01-002; Brewer et al., J Neurosci Res., 35(5):567-76, 1993) may be used to formulate a culture medium that comprises biotin, cholesterol, linoleic acid, linolenic acid, progesterone, putrescine, retinyl acetate, sodium selenite, tri-iodothyronine (T3), DL-alpha tocopherol (vitamin E), albumin, insulin and transferrin. The B27 Supplement supplied by PAA Laboratories GmbH comes as a liquid 50× concentrate, containing amongst other ingredients biotin, cholesterol, linoleic acid, linolenic acid, progesterone, putrescine, retinol, retinyl acetate, sodium selenite, tri-iodothyronine (T3), DL-alpha tocopherol (vitamin E), albumin, insulin and transferrin. Of these ingredients at least linolenic acid, retinol, retinyl acetate and tri-iodothyronine (T3) are nuclear hormone receptor agonists. B27 Supplement may be added to a culture medium as a concentrate or diluted before addition to a culture medium. It may be used at a 1× final concentration or at other final concentrations. Use of B27 Supplement is a convenient way to incorporate biotic, cholesterol, linoteic acid, linolenic acid, progesterone, putrescine, retinol, retinyl acetate, sodium selenite, tri-iodethyronine (T3), DL-alpha tocopherol (vitamin E), albumin, insulin and transferrin into a culture medium of the invention. It is also envisaged that some or all of these components may be added separately to the culture medium instead of using the B27 Supplement. Thus, the culture medium may comprise some or all of these components. In some embodiments, retinoic acid is absent from the B27 Supplement used in the culture medium, and/or is absent from the culture medium.

‘N2 Supplement’ is available from Invitrogen, Carlsbad, Calif.; www.invitrogen.com; catalog no. 17502-048; and from PAA Laboratories GmbH, Pasching, Austria; www.paa.com; catalog no. F005-004; Bottenstein & Sato, PNAS, 76(1):514-517, 1979. The N2 Supplement supplied by PAA Laboratories GmbH comes as a 100× liquid concentrate, containing 500 μg/ml human transferrin, 500 μg/ml bovine insulin, 0.63 μg/ml progesterone, 1611 μg/ml putrescine, and 0.52 μg/ml sodium selenite. N2 Supplement may be added to a culture medium as a concentrate or diluted before addition to a culture medium. It may be used at a 1× final concentration or at other final concentrations. Use of N2 Supplement is a convenient way to incorporate transferrin, insulin, progesterone, putrescine and sodium selenite into a culture medium of the invention. It is of course also envisaged that some or all of these components may be added separately to the culture medium instead of using the N2 Supplement. Thus, the culture medium may comprise some or all of these components. In some embodiments in which the medium comprises B27, it does not also comprise N2. In some embodiments, the final concentration of N-acetylcysteine is about 1 nM to about 100 nM, about 5 nM to about 50 nM, about 10 nM to about 50 nM, about 10 nM to about 30 nM, or about 25 nM.

In some embodiments the culture medium further comprises a ROCK inhibitor (Rho-Kinase inhibitor). A ROCK inhibitor is particularly useful for attachment of cells when establishing new cultures and/or when splitting (“passaging”) cells. Suitable ROCK inhibitors are known in the art and available commercially (including but not limited to GSK 269962, GSK 420286, H 1152 dihydrochloride, Glycyl-H 1152 dihydrochloride, SR 3677 dihydrochloride, SB 772077B dihydrochloride and Y-27632 dihydrochloride, all available from Tocris). In some embodiments, the ROCK inhibitor is at a final concentration of between 1 μM and 100 μM, between 1 μM and 50 μM, or between 5 μM and 20 μM. In some embodiments the ROCK inhibitor is Y-27632, optionally at a final concentration of about 10 μM.

In preferred embodiments, the culture medium does not contain thrombopoietin.

It is preferred that the culture medium does not comprise an undefined component such as fetal bovine serum or fetal calf serum. Various different serum replacement formulations are commercially available and are known to the skilled person. Where a serum replacement is used, it may be used at between about 1% and about 30% by volume of the medium, according to conventional techniques. In some embodiments, the culture medium is serum free.

The preferred culture methods of the invention are also advantageous because feeder cells are not required. Feeder cell layers are often used to support the culture of stem cells, and to inhibit their differentiation. The use of feeder cells is undesirable, because it complicates passaging of the cells (the cells must be separated from the feeder cells at each passage, and new feeder cells are required at each passage). The use of feeder cells can also lead to contamination of the desired cells with the feeder cells. This is clearly problematic for any medical applications, and even in a research context, complicates analysis of the results of any experiments performed on the cells.

Therefore, in some embodiments, the methods, media and compositions of the invention are feeder cell-free. A composition is conventionally considered to be feeder cell-free if the cells in the composition have been cultured for at least one passage in the absence of a feeder cell layer. A feeder cell-free composition of the invention will normally contain less than about 8%, less than about 4%, less than about 3%, less than about 2%, less than about 1% feeder cells (expressed as a % of the total number of cells in the composition) or preferably no feeder cells at all.

A culture medium of the invention will normally be formulated in deionized, distilled water. A culture medium of the invention will typically be sterilized prior to use to prevent contamination, e.g. by ultraviolet light, heating, irradiation or filtration. The culture medium may be frozen (e.g. at −20° C. or −80° C.) for storage or transport. The medium may contain one or more antibiotics to prevent contamination. The medium may have an endotoxin content of less than 0.1 endotoxin units per ml, or may have an endotoxin content less than 0.05 endotoxin units per ml. Methods for determining the endotoxin content of culture media are known in the art.

A preferred cell culture medium is a defined synthetic medium that is buffered at a pH of 7.4 (preferably with a pH 7.2-7.6 or at least 7.2 and not higher than 7.6) with a carbonate-based buffer, while the cells are cultured in an atmosphere comprising between 5% and 10% CO2, or at least 5% and not more than 10% CO2, preferably 5% CO2.

The invention also provides a composition or cell culture vessel comprising cells and/or organoids according to any one of the aspects of the invention described above, and a culture medium according to any one of the aspects of the invention described above. For example, such a composition or cell culture vessel may comprise any number of cells or organoids cultured according to a method of the invention, in a culture medium as described above.

According to a still further aspect of the invention, there is provided a hermetically-sealed vessel containing a culture medium of the invention. In some embodiments, the culture medium is an expansion medium. In some embodiments, the culture medium is a differentiation medium. Hermetically-sealed vessels may be preferred for transport or storage of the culture media, to prevent contamination. The vessel may be any suitable vessel, such as a flask, a plate, a bottle, a jar, a vial or a bag.

### Exemplary Culture Media for Use in the Invention

In some embodiments, the culture medium of the invention comprises an integrin agonist, one or more receptor tyrosine kinase ligands (e.g. EGF and/or HGF), a BMP inhibitor (e.g. Noggin) and a TCF-β inhibitor (e.g. A83-01). This culture medium optionally further comprises one or more Wnt agonists (e.g. an Lgr5 agonist). These media are suitable for all tissues, for example, intestine, gastric, pancreatic, liver, prostate, breast and lung.

In some embodiments, the culture medium of the invention comprises (i) FGF7 and/or FGF10, (ii) Noggin, (iii) an Lgr5 agonist and (iv) an integrin agonist. In some embodiments, the culture medium of the invention comprises: (i) FGF7 and/or FGF10, (ii) Noggin, (iii) an Lgr5 agonist, (iv) one or more further receptor tyrosine kinase ligands (for example, EGF) and (v) an integrin agonist. In some embodiments, the culture medium further comprises an ErbB3/4 ligand (e.g. human neuregulin ⊕-1). This is a culture medium that is particularly suitable for, but is not limited to, culturing breast stem cells.

In some embodiments, the culture medium of the invention comprises an integrin agonist, EGF, FGF (e.g. FGF 10), HGF, a TGF-β inhibitor (e.g. A83-01), nicotinamide, one or more Wnt agonists (e.g. an Lgr5 agonist), a cAMP pathway activator (e.g. forskolin) and gastrin. This culture medium optionally further comprises: (i) a BMP inhibitor (e.g. Noggin), a Wnt agonist (e.g. Wnt conditioned medium) and a Rock inhibitor (e.g. Y27632) or (ii) a BMP activator (e.g. BMP7). These culture media are particularly suitable for, but are not limited to, culturing liver stem cells.

In some embodiments, the culture medium of the invention comprises an integrin agonist, one or more receptor tyrosine kinase ligands (e.g. EGF), a BMP inhibitor (e.g. Noggin) and one or more Wnt agonists (e.g. an Lgr5 agonist). This culture medium optionally further comprises testosterone. These culture media are particularly suitable for, but are not limited to, culturing prostate stem cells.

In some embodiments, the culture medium of the invention further comprises one or more components selected from: a p38 MAP kinase inhibitor (for example, SB 202190), gastrin and/or nicotinamide.

In some embodiments, the culture medium of the invention further comprises a Rock inhibitor (e.g. Y27632). Addition of a Rock inhibitor has been observed to be useful for starting or splitting cultures.

In some embodiments, the culture medium of the invention further comprises B27 and/or N-acetylcysteine. These additional components are often added to a culture medium as components of a basal medium.

In some embodiments, the culture medium of the invention comprises an integrin agonist, an Lgr5 agonist, a BMP inhibitor (for example, Noggin), B27, N-acetylcysteine, Nicotinamide, a ROCK inhibitor a TGF-beta inhibitor (for example, A83-01), a p38 MAP kinase inhibitor (for example, SB 202190), FGF7 and FGF10, and optionally one or more additional components selected from: one or more further receptor tyrosine kinase ligands (for example, EGF, amphiregulin, TGF-alpha, PDGF), a p53 stabilising agent and a Wnt agonist (for example, Wnt3a).

In some embodiments, the culture medium of the invention comprises: (i) an integrin agonist, (ii) one or more receptor tyrosine kinase ligands (e.g. EGF and/or HGF), (iii) a BMP inhibitor (e.g. Noggin) and (iv) a TGF beta inhibitor (e.g. A83-01), a p38 inhibitor (e.g. SB202190) and/or a Rock inhibitor (e.g. Y-27632), and optionally further comprises one or more Wnt agonists (e.g. an Lgr5 agonist).

In some embodiments, the culture medium further comprises: (i) gastrin and/or nicotinamide; (ii) a Notch inhibitor (e.g. DAPT and/or DBZ) and/or (iii) a prostaglandin pathway activator (e.g. PGE2 and/or AA). For example, in some embodiments, the culture medium of the invention comprises: (i) an integrin agonist, (ii) one or more receptor tyrosine kinase ligands (e.g. EGF and/or HGF), (iii) a BMP inhibitor (e.g. Noggin) and (iv) gastrin, nicotinamide, a Notch inhibitor (e.g. DAPT and/or DBZ) and/or a prostaglandin pathway activator (e.g. PGE2 and/or AA).

In some embodiments, the culture medium further comprises a cAMP pathway activator (e.g. forskolin) and/or a BMP pathway activator (e.g. BMP7). In some embodiments, the culture medium comprises a BMP pathway activator (e.g. BMP7) and does not comprise a BMP pathway inhibitor (e.g. Noggin). These culture media are particularly suitable for, but are not limited to, culturing liver or pancreatic stem cells.

In some embodiments, the one or more receptor tyrosine kinase ligands is EGF and/or one or more ligands of FGFR2b (e.g. 1, 2, 3, 4, or more than 4), such as FGF7 and/or FGF10.

In some embodiments, the BMP inhibitor is Noggin.

In some embodiments, the one or more Wnt agonists is a Lgr5 agonist, Lgr4 agonist, Lgr6 agonist or Wnt3a. In some embodiments, the Lgr5 agonist is an Rspondin, e.g. any one of Rspondin 1-4.

In some embodiments, the culture medium further comprises an ErbB3/4 ligand (e.g. human neuregulin β-1).

When human epithelial stem calls are cultured, Wnt3a may advantageously be added to the culture medium.

In some embodiments, the culture medium is a differentiation medium comprising a basal medium and further comprising an integrin agonist, one or more receptor tyrosine kinase ligands (e.g. selected from EGF, FGF and HGF), a Notch inhibitor (e.g. DAPT), a glucocorticoid (e.g. dexamethasone), a TGF-beta inhibitor (e.g. A83-01) and one or more Wnt inhibitors (e.g. (i) a Porc inhibitor, optionally selected from IWP 2, LGK974 and IWP 1, and/or (ii) an inhibitor of β-catenin target gene expression, optionally selected from selected from iCRT3, CGP049090, PKF118310, PKF115 584, ZTM000990, PNU 74654, BC21, iCRT5, iCRT14 and FH535). In some embodiments the differentiation medium further comprises a GSK-3 inhibitor (e.g. CHIR99201). In some embodiments the differentiation medium further comprises an AP-1 stimulant (e.g. carbachol). Accordingly, in some embodiments the culture medium comprises EGF, FGF19, HGF, DAPT, IWP2, iCRT3, dexamethasone, CHIR99021 and carbachol.

In some embodiments, the culture medium is a differentiation medium comprising a basal medium and further comprising an integrin agonist, one or more EGFR pathway inhibitors (e.g. Gefitinib, Afatinib, a MEK inhibitor (e.g. PD0325901) and/or an ERK inhibitor (e.g. SCH772984)), a Notch inhibitor (e.g. DAPT) and one or more Wnt inhibitors (e.g. (i) a Porc inhibitor, optionally selected from IWP 2, LGK974 and IWP 1, and/or (ii) an inhibitor of β-catenin target gene expression, optionally selected from selected from iCRT3, CGP049090, PKF118310, PKF115 584, ZTM000990, PNU 74654, BC21, iCRT5, iCRT 14 and FH535).

For any of the culture media of the invention certain components can be left out for cancer cells.

### Organoids

The invention provides an organoid obtainable or obtained by a culture method of the invention. Thus, in some embodiments, the method of culturing an epithelial stem cell further comprises obtaining and/or isolating an organoid. As explained above, in some embodiments, the organoid is a tumour organoid. An organoid composing epithelial stem cells is also referred to herein as an “epithelial organoid”. Epithelial organoids are described in the prior art (e.g. see 022907, WO2010/090513, WO2012/014076, WO2012/168930, WO2015/173425, WO2016/083613, WO2016/083612 and WO2017/149025).

An epithelial organoid obtainable or obtained or cultured using a culture medium suitable for expansion comprises at least one epithelial stem cell, which can divide and produce further epithelial stem cells or can generate differentiated progeny. An epithelial organoid also comprises some differentiated (or more mature) cell types. These differentiated cell types arise spontaneously during formation of the organoid and contribute to the characteristic structural features of the organoid, as described below. Epithelial organoids are advantageous in that they maintain an expanding population of epithelial stem cells within the organoid, whist also containing more differentiated cell types characteristic of the epithelial tissue of origin. The length of time that the epithelial organoids can continue to be expand whilst maintaining a core presence of epithelial stem cells and whilst maintaining genotypic and phenotypic integrity of the cells, is an important feature of the epithelial organoids that distinguishes them from many of the organoids in the prior art. The epithelial organoids also have a distinctive structure that arises rapidly as the cells expand, differentiate and self-organise in vitro. These features are described in detail below. An epithelial organoid may also be subsequently cultured in a culture medium suitable for differentiation, as described herein, and this increase the proportion of differentiated cells in the epithelial organoid.

Image analysis may be used to assess characteristics of cells in culture such as cell morphology; cell structures; evidence for apoptosis or cell lysis; and organoid composition and structure. Many types of imaging analysis are well known in the art, such as electron microscopy, including scanning electron microscopy and transmission electron microscopy, confocal microscopy, stereomicroscopy, fluorescence microscopy. Histological analysis can reveal basic architecture and cell types.

In some embodiments, the epithelial organoid has a three dimensional structure, i.e. the organoid is a three-dimensional organoid. In some embodiments, the organoid comprises only epithelial cells, i.e. non-epithelial cells are absent from the organoid. This is because the culture media suitable for expending epithelial stem calls is specificity designed to expend epithelial stem cells. Therefore, even if other cell types are transiently present in the culture medium, e.g. in the tissue fragment that is the starting materiel of the invention, these cells are unlikely to survive and instead will be replaced by the longer term expansion of the stem cells which generate a pure population of epithelial cells.

In some embodiments, the epithelial cells in the organoid surround a lumen. In some embodiments, the organoid does not comprise a lumen (in particular the tumour organoids generally do not have a lumen). In some embodiments, the epithelial cells are polarized, (meaning that proteins are differentially expressed on the apical or basolateral side of the epithelial cell). In some embodiments the lumen is a sealed lumen (meaning that a continuous cellular barrier separated the contents of the lumen from the medium surrounding the organoid). In some embodiments the organoids comprise stem cells which are able to actively divide and which are preferably able to differentiate to all major differentiated cell lineages present in the corresponding in vivo tissue, e.g. when the organoid or cell is transferred in a differentiation medium. In some embodiments, the organoid comprises basal cells on the outside and more differentiated cells in the center.

In some embodiments, the organoids comprise stratified epithelium. By “stratified” it is meant that there are multiple (more than one) layers of cells. Such cells often tend to have their nuclei more central to the cells, i.e. not polarized. The cells in the multi-layer section may organise themselves to include a gap, or lumen between the cells.

In some embodiments the organoids comprise single monolayers that are folded (or invaginated) to form stem cell and differentiated cell compartments. In this embodiment, it is not necessary for there to be a central sealed lumen surrounded by an epithelial layer—instead the epithelium surrounding a three-dimensional organoid has effectively been unfolded and is presented as a single layer. It can sometimes be difficult to distinguish between folded (or invaginated) monolayers and regions of stratified cells. In some embodiments an organoid comprises both regions of stratified cells and regions of folded monolayers. In some embodiments the organoids of the invention have a section which is formed of multiple layers and a section comprising a single monolayer of calls. In some embodiments the organoids of the invention comprise or consist of a single monolayer of cells. In some embodiments, the organoid does not comprise a monolayer. The organoids according to the invention may possess a layer of cells with at least one bud and a central lumen.

In some embodiments the organoids of the invention comprise or consist of epithelial cells. In some embodiments, the organoids comprise or consist of a single layer of epithelial cells. In some embodiments non-epithelial cells are absent from the organoids.

In some embodiments, the organoid has been cultured or is capable of culture in expansion media of the invention for at least 2 months, for example at least 10 weeks, at least 12 weeks, at least 14 weeks, at least 16 weeks, at least 4 months, at least 5 months, at least 6 months, at least 9 months, at least one year.

In some embodiments, the organoid has been cultured or is capable of culture for at least 5 passages, at least 10 passages, at least 15 passages, or at least 20 passages. In some embodiments, the organoid or population of epithelial stem cells are cultured for at least 10 passages.

In some embodiments, the cell number of the organoid increases exponentially over at 6 passages, 10 passages, 15 passages, or 20 passages. In a preferred embodiment, the cell number of the organoid or population of epithelial stem cells increases exponentially over 5 passages.

In some embodiments, an organoid is at least 50 μm, at least 60 μm, at least 70 μm, at least 80 μm, at least 90 μm, at least 100 μm, at least 125 μm, at least 150 μm, at least 175 μm, at least 200 μm, at least 260 μm or more in diameter at the widest point.

Within the context of the invention, a tissue fragment is a part of an adult tissue, preferably a human adult tissue. An organoid, by contrast, develops structural features through in vitro expansion, and is therefore distinguished from a tissue fragment.

In a preferred embodiment an organoid could be cultured during at least 2, 3, 4, 5, 6, 7, 8, 9, 10 weeks or 1, 2, 3, 4, 5, 6, 7, 8, 9, 10 months or longer. In some embodiments, the organoid is expanded or maintained in culture for at least 3 months, preferably at least 4 months, at least 5 months, at least 6 months, at least 7 months, at least 9 months, or at least 12 months or more. Advantageously, use of the culture methods provided by the present invention results in organoids and/or cell populations being formed in which the number of chromosomes remains stable when the cells or organoids are cultured long-term. Thus, in some embodiments, the organoids or population of epithelial stem cells of the invention has a stable chromosome member after 2, 4, 6, 8, 10, 12 or 14 weeks or after 4, 5, 6 or more months in culture in an culture medium of the invention. Preferably, at least 35%, at least 70%, more preferably at least 75%, more preferably at least 80%; more preferably at least 90%, more preferably at least 95%, more preferably at least 99% of the cells have the correct number of chromosomes after 2, 4, 6, 8, 10, 12 or 14 weeks or after 4, 5, 6 or more months culture in an culture medium of the invention. For human epithelial cells, the correct number of chromosomes is 46. In some embodiments, the organoid has a normal karyotyoe. One method of determining the karyotype is by metaphase spread analysis. Of course, it is to be understood that tumour cells in tumour organoids, e.g. derived from tumour stem cells, would not necessarily have the correct number of chromosomes as genomic instability is a feature of certain cancers.

In some embodiments, an organoid of the invention is a tumour organoid. In some embodiments, tumour organoids are dense structures, similar to the resultant organoids from the culture of non-cancerous epithelial stem cells. In other embodiments, tumour organoids are cystic structures. In some embodiments, tumour organoids derived from different patients show different morphologies. Organoid morphology may be assessed using techniques such as image analysis, including brightfield microscopy.

In some embodiments, tumour organoids of the invention retain tumour-specific histopathological changes or characteristics. Such changes can be identified by comparing organoids derived from the tumour tissue and the adjacent epithelium from the same patient and/or by comparing the tumour organoid with the primary tissue specimen.

In some embodiments, the tumour organoid is a dense or cystic structure. In some embodiments, the tumour organoid comprises transformed epithelial tumour cells. In some embodiments, the tumour organoids predominantly comprise transformed epithelial tumour cells. In some embodiments, the tumour organoids contain only transformed epithelial tumour cells (i.e. non-transformed epithelial cells are absent from the tumour organoid). In some embodiments, the tumour organoid does not comprise immune-, connective tissue- and/or vessel-elements. The skilled person would appreciate that the presence of transformed epithelial tumour cells could be assessed by various methods, including keratin (KRT5) immuno-staining.

In some embodiments, tumour organoids of the invention are selected by culture in a culture medium of the invention that further comprises an Mdm2 agonist, such as Nutlin-3. The Mdm2 agonist may be present for the whole of or part of the culture process. In some embodiments, Nutlin-3 is present at a concentration of about 10 μM. Nutlin-3 prevents the growth of p53 wild type cells. P53 is a tumour suppressor gene. Thus decreased p53 function, for example caused by a mutation in the p53 gene or other mis-regulation of p53, is a common cause of tumorigenesis. Culturing organoids in the presence of Nutlin-3 therefore selected organoids with decreased levels of p53 or deceased p53 activity and can be used as a method for selecting tumour organoids.

In some embodiments, organoids and tumour organoids of the invention have different transcriptome profiles. In some embodiments, organoids of the invention cluster together and tumour organoids of the invention cluster together based on a principle component analysis of a transcriptome analysis. In some embodiments, organoids and tumour organoids of the invention have differential gene expression. For example, KLK6 and/or EHF are downregulated in HNSCC tumour organoids. Whereas, SLCOB1, HOXC13, CALB1, NTS and/or BCHE are upregulated in HNSCC tumour organoids. Further genes which may show differential expression between non-tumour organoids and tumour organoids include SDC2, HOXA1, NXPE3 and/or HOXC10. In some embodiments, when corresponding non-tumour organoids and tumour organoids are compared there are more than 50 differentially expressed genes, more than 100 differentially expressed genes, more than 200 differentially expressed genes or more than 300 differentially expressed genes.

In some embodiments, a tumour organoid of the invention has an enrichment of the variable allele frequency of detected mutations relative to the tumour. Variant allele frequency refers to the frequency that a mutation is detected within a sample, for example this can be given as the percentage of reads from a sequencing analysis that contain a particular mutation.

In some embodiments, the genetic alienations in a tumour organoid of the invention recapitulate the genetic alterations of the tumour from which the organoid is derived. Genetic alterations include single nucleotide variant and small insertions and deletions. In some embodiments, organoids of the invention which are not derived from cancer cells contain fewer genetic alterations than tumour organoids of the invention which are derived from cancer cells.

Chromosome mis-segregation underlies the aneuploidies frequently observed in human tumours. Chromosome segregation errors include anaphase bridges and bi-nucleated cells undergoing multipolar division. Increase mis-segregation rates result in the phenotype known as chromosomal instability, which is commonly observed in cancers. In some embodiments, tumour organoids demonstrate chromosome instability. In some embodiments, the tumour organoid has an increased rate of chromosome segregation errors compared to organoids derived from cells which are not cancer cells. In some embodiments, the tumour organoid has an increased rate of chromosome segregation errors, including anaphase bridges and/or bi-nucleated cells undergoing multi-polar division.

In some embodiments, a tumour organoid of the invention does not have a normal karyotype. In some embodiments, the tumour organoid displays aneuploidy. The karyotype of an organoid, i.e. the number of chromosomes, can be determined by metaphase spread analysis.

In some embodiments, a tumour organoid of the invention is tumourogenic when subcutaneously transplanted. The tumourogenic potential of human organoids can be determined by subcutaneous transplantation of said organoids into mice. Typically the subcutaneous transplantation of non-tumour organoids does not result in tumour formation or at least outgrowth of the organoid. In some embodiments, a tumour organoid of the invention retains its tumourigenic potential in culture and can form tumours with similar features to the parental tumour following subcutaneous transplantation. Such characteristics may include levels of atypia that are regarded cancerous, tripolar mitotic figures, nuclear polymorphism and/or muscle invasion.

The expansion organoid of the invention preferably comprises at least 50% viable cells, more preferred at least 60% viable cells, more preferred at least 70% viable cells, more preferred at least 80% viable cells, more preferred at least 90% viable cells. Viability of cells may be assessed using Hoechst staining or Propidium Iodide staining in FACS. In some embodiments, there is provided one or more frozen organoids of the invention. Also provided is a method for preparing organoids for freezing comprising dissociating organoid cultures and mixing them with a freezing medium such as Recovery cell culture freezing medium (Gibco) and freezing following standard procedures. A method for thawing frozen organoids is also provided which comprises thawing frozen organoids, embedding the thawed organoids in an extracellular matrix (e.g. Matrigel) and culturing the organoids in an culture medium of the invention. Advantageously, initially after thawing the culture medium may be supplemented with Y-27632, for example, about 10 uM Y-27632. In some embodiments, the culture medium is supplemented with Y-27632 for the first 1, 2, 3, 4, 5 or less days after thawing, preferably for the first 3 or 4 days. In some embodiments, Y-27632 is not present in the culture medium after the first 3, 4, 5, 6 or more days, preferably after the first 3 or 4 days.

This freezing method can be used for expansion organoids of the invention. In some embodiments, the organoid or tumour organoid of the invention is cryopreserved.

### Differentiation

Organoids, or cells from the organoids can be transferred to a differentiation medium and be allowed to or induced to differentiate into all major differentiated cell lineages.

In some embodiments, the method of the invention comprises a first step of culturing an epithelial stem cell or population of stem cells in an expansion medium and a second step of culturing the expanded cells or expansion organoid in a differentiation medium. In some embodiments the integrin agonist is included in the culture medium in only one of these steps. In other embodiments, the integrin agonist is included in the culture medium in both of these steps.

In some embodiments, the differentiation medium comprises a culture medium as disclosed herein from which one or more of the following are not included: a mitogenic growth factor, a TGF-beta inhibitor, an activator of the prostaglandin signalling pathway, a Wnt agonist, a cAMP pathway activator, a BMP inhibitor and nicotinamide (e.g. as described in WO2012/168930). In some embodiments, the differentiation medium comprises a basal medium for animal or human cells. In some embodiments, the differentiation medium further comprises one or more of an EGFR pathway inhibitor, a Notch inhibitor, a Wnt inhibitor or a BMP pathway activator (e.g. as described in WO2017220586).

In some embodiments, culturing the population of epithelial stem cells in the differentiation medium increases expression levels of mature epithelial cell markers. The skilled person is aware of different techniques to determine gene or protein expression, including quantitative PCR.

### Uses of Organoids

Organoids of the invention faithfully represent the in vivo situation. This is true both for organoids grown from normal tissue and for organoids grown from diseased tissue. Therefore, organoids of the invention are useful in medicine and diagnostics, and in research and drug development. As well as providing normal ex vivo cell/organ models, the organoids of the invention can be used as ex vivo models of disease and/or infection. Therefore, organoids of the invention can be used for drug screening, including drug discovery and validation, target discovery and validation, toxicology, infection models and other research purposes. Diseases that can be studied by the organoids of the invention include but are not limited to genetic diseases, metabolic diseases, pathogenic diseases and inflammatory diseases. The organoids are also suitable for transplantation, and thus have potential for regenerative medicine. In addition, organoids can be grown quickly from any individual's cells, and thus can be used for identifying suitable treatment regimes, e.g. in the context of personalised medicine. Several uses of organoids are described in earlier applications (e.g. in WO2009/022907, WO2010/090513, WO2012/014076, WO2012/168930, WO2015/173425, WO2016/083613, and WO2016/083612) and these uses also apply to the organoids of the invention.

The examples provide details on the properties of the organoids that make them suitable for these uses, and further specific examples are provided below.

### Drug Screening

The invention provides the use of an organoid (or cells directly obtained from said organoid) in drug screening, target validation, target discovery, toxicology or a toxicology screen.

The cells are preferably exposed to multiple concentrations of a test agent for a certain period of time. At the end of the exposure period, the cultures are evaluated. The organoid can also be used to identify drugs that specifically target epithelial carcinoma cells. It will be understood by the skilled person that the organoids of the invention would be widely applicable as drug screening tools for infectious, inflammatory and neoplastic pathologies. In some embodiments, the invention provides the use of an organoid in drug screening, target validation, target discovery, toxicology, toxicology screens or an ex vivo cell/organ model. In some embodiments, the invention provides the use of an organoid in an ex vivo method to predict a clinical outcome. In some embodiments, the organoids of the invention could be used for screening for cancer drugs.

In some embodiments, the organoids of the invention can be used to test libraries of chemicals, antibodies, natural product (plant extracts), etc for suitability for use as drugs, cosmetics and/or preventative medicines. For instance, in some embodiments, a cell biopsy from a patient of interest, such as tumour cells from a cancer patient, can be cultured using culture media and methods of the invention and then treated with a chemical compound or a chemical library. It is then possible to determine which compounds effectively modify, kill and/or treat the patient's cells. This allows specific patient responsiveness to a particular drug to be tested thus allowing treatment to be tailored to a specific patient. Thus, this allows a personalised medicine approach. In some embodiments, the drug screening method is an ex vivo method to guide personalised theory and/or predict a clinical outcome. In some embodiments, the invention provides an organoid for use in a method to guide personalised therapy. The added advantage of using the organoids for identifying drugs in this way is that it is possible to screen normal organoids (organoids derived from healthy tissue) to check which drugs and compounds have minimal effect on healthy tissue. This allows screening for drugs with minimal off target activity or unwanted side effects.

In some embodiments, the invention provides a method for testing the effect of a candidate compound, wherein the method comprises:
culturing epithelial stem cell or a population of epithelial stem cells according to the method of the invention, optionally for less than 21 days;
exposing the resultant population of cells or the resultant organoid to one or a library of candidate compounds;
evaluating said expanded organoids for any effects,
identifying the candidate molecule that causes said effects as a potential drug; and optionally
providing said candidate molecule, e.g. as a drug.

In some embodiments, the method for testing the effect of a candidate compound comprises exposing the organoid to radiation in the presence or absence of a candidate compound. In some embodiments, an evaluated effect in the method for testing the effect of a candidate compound is selected from the list comprising: a reduction in, or loss of, proliferation, a morphological change, cell death or a change in gene or protein expression.

A library of candidate molecules comprises more than one candidate molecule.

In some embodiments, the invention provides a method comprising:
culturing epithelial stem cell or a population of epithelial stem cells according to the method of the invention, optionally for less than 21 days;
exposing the resultant organoid or a population of cells derived from the resultant organoid to a treatment, such as radiation, and/or to one or a library of candidate molecules;
evaluating said organoid or population of cells for any effects of a candidate molecule; and
correlating said effect with a feature of the organoid, for example the presence of one or more genetic mutations, such as mutations in the EGFR signalling pathway, including PIK3CA, KRAS, HRAS or BRAf.

In some embodiments, the invention provides a method comprising:
culturing epithelial stem cell or a population of epithelial stem cells according to the method of the invention, optionally for less than 21 days;
exposing the resultant organoid or a population of cells derived from the resultant organoid to a treatment, such as radiation, and/or to one or a library of candidate molecules;
evaluating said organoid or population of cells for any effects of a candidate molecule;
comparing said effect with standard values and/or previous observations; and optionally
predicting clinical outcome and/or selecting a personalised medicine.

In some embodiments, the organoid is derived from a patient biopsy. In some embodiments, the candidate molecule that causes a desired effect on the organoid or population of cells derived from said organoid is administered to said patient. Accordingly, in one aspect, there is provided a method of treating a patient comprising:
a) obtaining a biopsy from the diseased tissue of interest in the patient;
b) culturing the biopsy to obtain an organoid, preferably using a method for culturing epithelial stem cells as described herein;
c) identifying a suitable drug using a screening method of the invention; and
d) treating said patient with the drug obtained in step (c).

In some embodiments, the invention provides a method for selecting a treatment regime for a patient, wherein the method comprises the steps of:
optionally obtaining a biopsy from the tissue of the patient;
culturing the biopsy, a tissue fragment of the biopsy, an epithelial stem cell of the biopsy or a population of epithelial stem cells of the biopsy to obtain an organoid, preferably using a method for culturing epithelial stem cells as described herein;
exposing the resultant organoid to a treatment regime, including radiation and/or one or more candidate compounds;
evaluating said organoid for any effects;
identifying the treatment of regime that causes said affects; and
optionally providing said treatment to the patient.

### Transplantation and Medicine

The invention provides the use of organoids in regenerative medicine and/or transplantation. The invention also provides methods for treatment wherein the method comprises transplanting an organoid into an animal or human.

In some embodiments, the invention provides an organoid for use in diagnostics or medicine. In some embodiments, the invention provides an organoid for use in diagnostics or medicine, optionally in personalised medicine or diagnostics, or regenerative medicine. In some embodiments, the invention provides method of treating a disease comprising the step of administering an organoid of the invention. In some embodiments, the invention provides the use of an organoid of invention in the manufacture of a medicament for treating a disease.

### Uses of Integrin Agonists

The invention provides the use of an integrin agonist as defined herein for culturing cells, optionally using any culture method and/or culture medium described herein.

It may also be useful to contact cells in vitro with an integrin agonist as defined herein prior to transplantation of the cells into a subject. Therefore, the invention provides the use of an integrin agonist for pro-treating cells prior to transplantation into a subject.

It may also be useful to administer the integrin agonist directly to a subject to improve cell transplantation. Therefore, the invention also provides an integrin agonist as defined herein for use as a cell adhesion-enhancer in a method of cell transplantation. The invention also provides a method for transplanting cells into a subject, wherein the method comprises administration of an integrin agonist as defined herein to the subject.

The pro-treatment with or administration of the integrin agonist in this context is expected to enhance cell adhesion and to improve the success rate of the transplantation therapy. This may be useful in regenerative medicine, e.g. for the treatment of liver disease, diabetes or any other disease that may benefit from cell transplantation therapy.

These uses of the integrin agonist are expected to be advantageous for all cell types and not only for epithelial stem cells. In particular, it is expected to be advantageous for stem cells, including hematopoietic stem cells, embryonic stem cells or induced pluripotent stem cells. The use of the integrin agonist is expected to be particularly useful in hematopoietic stem cell culture—even though hematopoietic stem cells have been used routinely in many hospitals for many decades, it has not yet been possible to usefully expand these cells in culture. Therefore, in some embodiments, the cell is a hematopoietic stem cell. Therefore, in some embodiments, the cell is a stem cell, optionally an epithelial stem cell, a hematopoietic stem cell, an embryonic stem cell or an induced pluripotent stem cell.

### Definitions

As used herein, the verb “to comprise” and its conjugations is used in its non-limiting sense to mean that items following the word are included, but items not specifically mentioned are not excluded. In addition the verb “to consist” may be replaced, if necessary, by “to consist essentially of” meaning that a product as defined herein may comprise additional component(s) than the ones specifically identified, said additional component(s) not altering the unique characteristic of the invention. In addition a method as defined herein may comprise additional step(s) than the ones specifically identified, said additional step(s) not altering the unique characteristic of the invention. In addition, reference to an element by the indefinite article “a” or “an” does not exclude the possibility that more than one of the element is present unless the context clearly requires that there be one and only one of the elements. The indefinite article “a” or “an” thus usually means “at least one”.

As used herein, the term “about” or “approximately” means that the value presented can fee varied by +/−10%. The value can also be read as the exact value and so the term “about” can be omitted. For example, the term “about 100” encompasses 90-110 and also 100.

All patent and literature references cited in the present specification are hereby incorporated by reference in their entirety.

Any reference to a method for treatment comprising administering an agent to a patient, also covers that agent for use in said method for treatment, as well as the use of the agent in said method for treatment, and the use of the agent in the manufacture of a medicament.

The following examples are offered for illustrative purposes only, and are not intended to limit the scope of the present invention in any way.

### Embodiments

The invention includes the following numbered embodiments.
1. A method for culturing an epithelial stem cell or an organoid comprising epithelial stem cells, wherein the method comprises culturing said epithelial stem cell in a culture medium suitable for epithelial stem cells, wherein the culture method further comprises contacting the cell or organoid with an integrin agonist.
2. The method of embodiment 1, wherein the integrin agonist interacts with the beta subunit of integrin.
3. The method of embodiment 2, wherein the beta subunit is β1, β2, or β7.
4. The method of embodiment 3, wherein the integrin agonist interacts with the β1 subunit.
5. The method of embodiment 1, wherein the integrin agonist interacts with the alpha subunit of integrin.
6. The method of any one of embodiment 1-5 wherein the integrin agonist is selected from an anti-integrin antibody, a talin, a kindlin, dithiothreitol and oxysterol 25-hydroxycholesterol.
7. The method of any one of embodiments 1-6, wherein the agonist of integrin is an anti-integrin antibody.
8. The method of embodiment 7, wherein the anti-integrin antibody is JBS2, HP1/3, SNAKA51, PT25-2, PMI-1, MEM-83, NKI-L16, 496B, 12G10, 8A2, TS2/16, 15/7, HUTS-4, 8E3, N29, 9EG7, mAB 24, MEM-148, KIM127, CBR LFA-1/2, MEM-48, KIM185, AP3, AP5, LIBS6, LIBS2, 10F8, 2B8, 2G3.
9. The method of embodiment 8, wherein the anti-integrin antibody is TS2/16, 12G10, 8A2, 15/7 HUTS-4, 8E3, N29 or 9EG7.
10. The method of embodiment 9, wherein the anti-integrin antibody is TS2/16, 12G10 or 8A2.
11. The method of any one of embodiments 7-10, wherein the antibody is humanised.
12. The method of any one of embodiments 1-6, wherein the integrin agonist is a talin.
13. The method of embodiment 12, wherein the talin is used in combination with a kindlin.
14. The method of any one of embodiments 1-6, wherein the integrin agonist is a reducing agent, such as dithiothreitol.
15. The method of any one of embodiments 1-6, wherein the integrin agonist is a lipid, such as oxysterol 25-hydroxycholesterol.
16. The method of any one of embodiments 1-15, wherein the method results in the growth of an organoid.
17. The method of any one of embodiments 1-16, wherein the method results in an at least 10%, 20%, 50% increase in epithelial stem cell growth over 4 days relative to the same method performed without the integrin agonist.
18. The method of any preceding embodiment, whereas the method further comprises culturing the cells in contact with an extracellular matrix.
19. The method of embodiment 18, wherein the extracellular matrix to a Basement Membrane Extract or Matrigel.
20. The method of any proceeding embodiment, wherein the method further comprises culturing the cells in contact with a synthetic matrix.
21. The method of embodiment 20, wherein the synthetic matrix comprises a polymer, optionally a polyester, polyethylene glycol or a hydrogel.
22. The method of embodiment 20 or embodiment 21, wherein the synthetic matrix comprises a cross-linked polyethylene glycol (PEG) hydrogel.
23. The method of embodiment 20-22, wherein the synthetic matrix comprises a biomaterial, preferably an extracellular matrix component.
24. The method of embodiment 23, wherein the biomaterial is one or more glycoprotein (optionally selected from collagen, laminin, perlecan, fibronectin or an RGD attachment ligand of fibronectin) and/or one or mote carbohydrate (optionally hyaluronic acid).
25. The method of any one of the preceding embodiments, wherein the extracellular matrix or synthetic matrix is three-dimensional.
26. The method of any one of the preceding embodiments, wherein the extracellular matrix or synthetic matrix is in suspension.
27. The method of any one of embodiments 1-17 and 20-26, wherein the culture method does not involve contacting the cells with an exogenous extracellular matrix.
28. The method of any one of the preceding embodiments, wherein the epithelial stem cell is part of a population of epithelial stem cells, optionally comprised in an organoid and/or an epithelial tissue explant.
29. The method of any one of the preceding embodiments, wherein the method comprises co-culture with a no-epithelial cell type, optionally an immune cell.
30. The method of any one of the preceding embodiments, wherein the epithelial stem cell is a mammalian cell.
31. The method of embodiment 38, wherein the epithelial stem cell is a human cell.
32. The method of any one of the preceding embodiments, wherein the epithelial stem cell is selected from a colorectal, small intestine, stomach, pancreas, liver, lung, breast, prostate, kidney, mouth, nasopharynx, throat, hypopharynx, larynx, trachea, skin, fallopian tube, ovary, salivary gland, esophagus, hair follicle and/or cochlear cell.
33. The method of any one of the preceding embodiments, wherein the epithelial stem cell is a colorectal cell.
34. The method of any one of the preceding embodiments, wherein the culture medium suitable for epithelial stem cells comprises one or more of a Wnt agonist, a BMP inhibitor, a mitogenic growth factor and a TGF-beta inhibitor.
35. The method of embodiment 34, wherein the culture medium suitable for epithelial stem cells comprises a Wnt agonist.
36. The method of embodiment 35, wherein the culture medium suitable for epithelial stem cells further comprises a mitogenic growth factor and/or a BMP inhibitor.
37. The method of embodiments 34-36, wherein the culture medium suitable for epithelial stem cells comprises a Wnt agonist, a BMP inhibitor, a mitogenic growth factor and a TGF-beta inhibitor.
38. The method of embodiments 34-37, wherein the Wnt agonist is selected from one or more of an R-spondin family protein, a Wnt ligand from the Wnt family of secreted glycoproteins, an inhibitor of intracellular β-catenin degradation, a GSK-3 inhibitor, an activator of TCF/LEF, and an inhibitor of RNF43 or ZNRF3.
39. The method of embodiments 34-38, wherein the mitogenic growth factor binds to a receptor tyrosine kinase such as EGFR, an FGFR or HGFR.
40. The method of embodiments 34-39, wherein the mitogenic growth factor is one or more of EGF, FGF and HGF.
41. The method of embodiments 34-40, wherein the BMP inhibitor is noggin.
42. The method of embodiments 34-41, wherein the TGF-beta inhibitor is an inhibitor of the ALK4, ALK5 or ALK7 signaling pathway.
43. The method of embodiments 34-42, wherein the TGF-beta inhibitor is a small molecule inhibitor such as A83-01.
44. The method of embodiments 34-43, wherein the culture medium suitable for epithelial stem cells further comprises one or more of nicotinamide, gastrin, B27 and N-acetylcysteine.
45. The method of any preceding embodiment, wherein the culture medium suitable for epithelial stem cells is suitable for expanding epithelial stem cells.
46. The method of any preceding embodiment, wherein the culture medium suitable for epithelial stem cells is suitable for differentiating epithelial stem cells.
47. A culture medium as defined in any one of embodiments 34-46, wherein the culture medium further comprises an integrin agonist as defined in embodiments 2-15.
48. An extracellular matrix or a synthetic matrix as defined in any one of embodiments 18-27, wherein the matrix further comprises an integrin agonist as defined in embodiments 2-15.
49. An organoid obtainable or obtained by a method of any one of embodiments 1-46.
50. The organoid of embodiment 49, wherein the organoid has a normal karyotype.
51. The organoid of embodiment 43 or embodiment 50, wherein the organoid has a rosette-like structure.
52. The organoid of embodiment 49, wherein the organoid is tumour organoid.
53. A composition comprising a culture medium according to embodiment 47, and an organoid according to any one of embodiments 40-52.
54. A composition comprising a culture medium of 47, and an extracellular matrix or a synthetic matrix, optionally as defined in any one of embodiments 18-27.
55. Use of an integrin agonist for culturing a cell.
56. Use of an integrin agonist for pre-treating cells prior to transplantation into a subject.
57. An integrin agonist for use as a cell adhesion-enhancer in a method of cell transplantation.
58. The use according to embodiment 53 or embodiment 54, or the integrin agonist for use according to embodiment 56, wherein the cell is a stem cell, optionally an epithelial stem cell, a hematopoietic stem cell, an induced pluripotent stem call or an embryonic stem cell.
59. Use of an organoid according to any one of embodiments 49-52, for drug screening, target validation, target discovery or toxicology.
60. An organoid according to any one of embodiments 49-52, for use in therapy or for use in diagnosis.

### EXAMPLES

### Example 1

### The Production of a Humanised TS2/16 Antibody

A humanised TS2/16 antibody was produced by U-Protein Express BV, Utrecht, The Netherlands. This involved the generation of coding sequences of the antibody variable domains via synthetic gene design and codon optimization. Generating an expression vector by ligation of the antibody variable domain synthetic fragments into antibody expression vectors using BsmBI restriction sites on the 5′ and 3′ ends, in frame with the constant region of the Heavy and Light chain. Transiently producing the antibody expression vectors in HEK293 cells or CHO cells via the rPEx technology, and then subsequently purifying the recombinant antibody via affinity chromatography (protein A), ion exchange chromatography and/or and gel filtration chromatography.

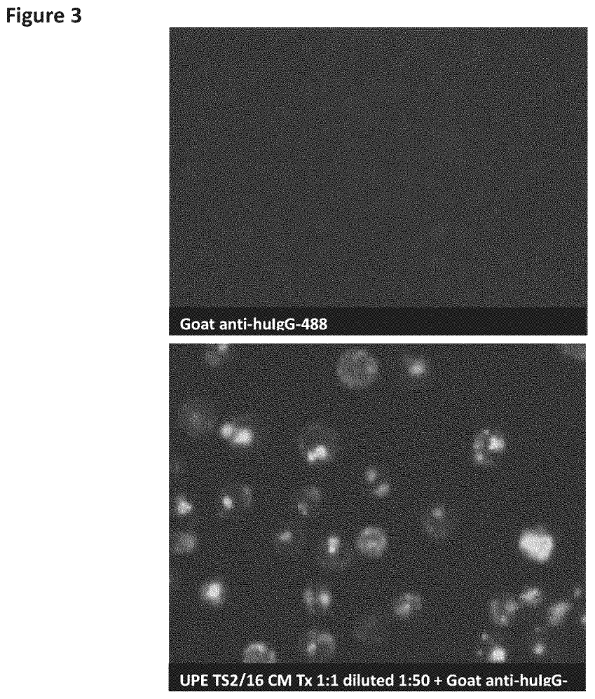

The humanised antibody was analysed using NU-PAGE Tris-Acetate Gel/SDS buffer system (Invitrogen) under non-reducing conditions and the resulting NuPAGE gel can be seen in FIG. 2. Transfection of expression plasmids for the Heavy-, and L-chain in ratio's of 1:1 and 1:1.5 produced stronger protein bands on the gel than a ratio of 1:3. The heavy and light chains of the humanised antibody were sequenced and found to be SEQ ID NOs 3 and 4, respectively. The CDRs regions of the heavy and light chains were identified as SEQ ID Nos 5-7 and 8-10, respectively

### Example 2

### Testing the Integrin β1 Recognition by the Humanised TS2/16 Antibody

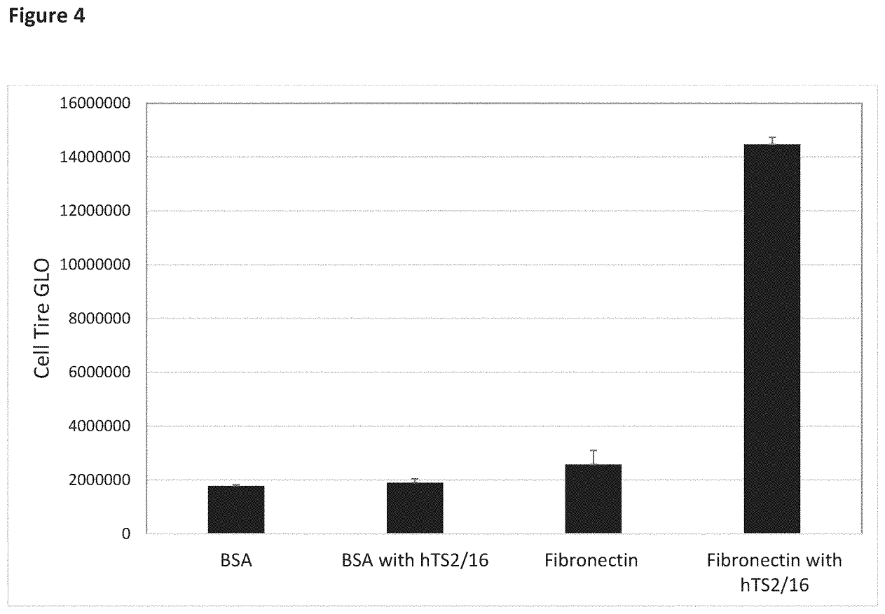

K562 cells (a human immortalised myelogenous leukemia cell line) were incubated with the humanised TS2/16 antibody in conditioned medium. The human erythroleukemic cell line K562 expresses only α5β1 as β1 class of integrin on the surface [17]. Antibody binding was visualized using by incubating the cells with a Goat anti-hulgG1 conjugated to Alexa 488 (Life Technologies (A11013) in PBS 1% BSA at a dilution of 1:250 (FIG. 3). The results demonstrate that the antibody successfully binds to integrin on the surface of the cells.

### Example 3

### Confirming the Functional Activity of the Humanised TS2/16 Antibody

The ability of the humanised TS2/16 antibody to induce cell adhesion to fibronectin was tested as described in [17]. Briefly 96-well plates were coated overnight with either BSA or Fibronectin (5 μg/ml in PBS). Any non-specific binding sites were subsequently blocked for 30 minutes with 1% BSA in PBS. Human erythroleukemic cell line K562 cells (10⁵ cells/well) were incubated 1 hr at 37° C. with in the presence or absence of humanised TS2/16. The experiments were performed in triplicate. The unbound cells were removed by rinsing the wells twice with PBS. The bound cells were quantified using the cellular ATP-driven CellTiterGlo assay (Promega). Briefly, the CellTiterGlo® Assay determines the number of viable cells in culture based on quantitation of the ATP present, which signals the presence of metabolically active cells.

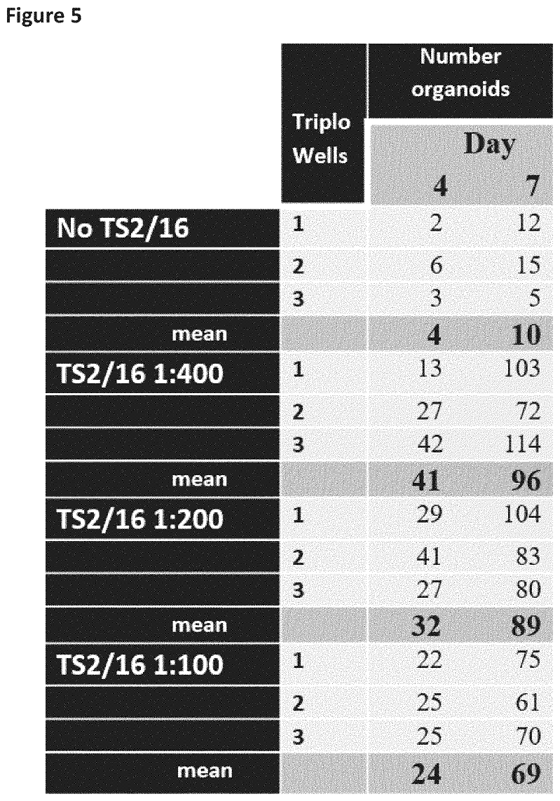

FIG. 4 demonstrates that the humanised TS2/16 antibody can induce cell adhesion to Fibronectin as have previously been demonstrated for non-humanised versions of the antibody, but not to the control BSA.

A similar experiment was performed with the MAB1778 antibody, which is another anti-integrin agonist antibody that targets the β1 integrin subunit MAB1778 was also able to induce adhesion of these cells.

### Example 4

### Culturing Epithelial Stem Cells in Culture Medium Without Any Extracellular Matrix Components

The inventors tested whether the addition of an integrin agonist would allow epithelial stem cells growth in a culture medium that does not comprise any exogenously added extracellular matrix components. As mentioned previously, the presence of the extracellular matrix in a culture medium is a known requirement for efficient epithelial stem cell growth.

Single human epithelial colon stem cells were used as described in [18]. The single human epithelial colon stem cells were plated into Corning UltraLowAttachment 96 well flat bottomed plates containing a culture medium as described below. The Corning Ultra-Low Attachment surface is a hydrophilic, neutrally charged coating covalently bound to a polystyrene vessel surface. The hydrogel inhibits specific and nonspecific immobilization, forcing cells into a suspended state, enabling 3D spheroid formation. The coating is stable, noncytotoxic, biologically inert, and non-degradable.

The culture medium contained advanced DMEM/F12 medium including B27, nicotinamide, N-acetylcysteine, noggin, R-spondin 1, EGF, a Wnt Surrogate [19] TGf-b type I receptor inhibitor A83-01 and P38 inhibitor SB202190. Notably the culture medium did not contain an extracellular matrix.

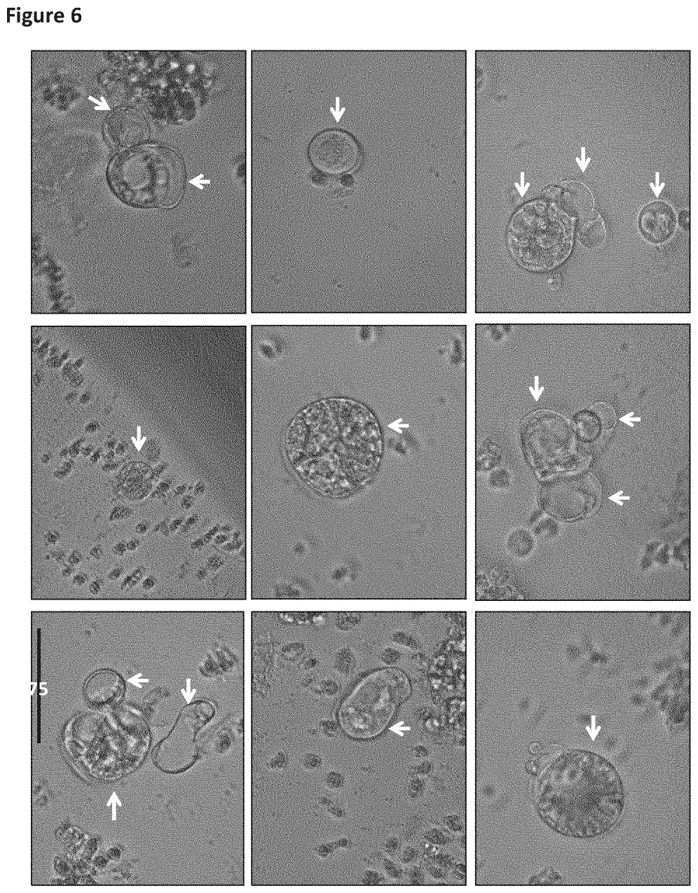

The single human epithelial colon stem cells were then cultured in human colon organoid medium supplemented with TS2/16-conditioned medium (UPE) at dilutions of 1:100, 1:200 and 1:400 or without the antibody. Three replicates were performed for each treatment condition. The growth rate of the epithelial stem cells was measured on days 4 and 7 post treatment by counting the total number of organoids (FIG. 5).

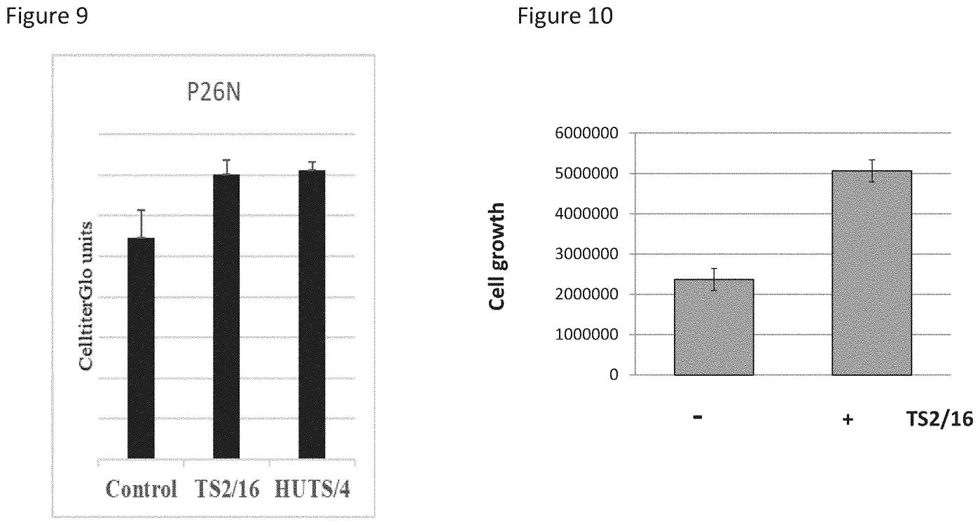

The addition of an integrin agonist resulted in an increase in epithelial stem cell growth compared to the culture medium that did not contain the humanised TS2/16 antibody (see FIG. 5). The optimal effect of the integrin agonist was observed with the lowest dilution 1:400, but the increased growth rate was seen across all the dilutions tested. Examples of the growing organoids in the culture medium comprising the humanised TS2/16 antibody con be seen in FIG. 8.

The inventors have surprisingly demonstrated that the addition of an integrin agonist to a culture medium which does not contain any extracellular matrix components can successfully result in efficient epithelial stem cell growth. As observed in the negative control, in the absence of any extracellular matrix components the epithelial stem cells do not significantly grow. Therefore, a culture method comprising contracting a cell or organoid with an integral agonist can be used to in improved methods for culturing an epithelial stem cell or an organoid.

The inventors hypothesise that the integrin agonist is able to mimic the structural and signalling functions of the extracellular matrix, because the addition of an integrin agonist was surprisingly able to allow epithelial stem cell growth in the absence of the extracellular matrix. Without wishing to be bound by any particular theory, the integrin agonist may alter the conformation state of integrin, thereby activating it to integrin ligands. The integrin agonist then also mimics the action of an integrin ligand which activates signalling pathways and promotes epithelial stem cell growth.

### Example 5

### The Addition of a Humanised TS2/16 Antibody to a Culture Medium Comprising an Extracellular Matrix

Colon epithelial stem cells were isolated as described in Example 4. The single cells were cultured in medium comprising advanced DMEM/F12 medium including B27, nicotinamide, N-acetylcysteine, noggin, R-spondin 1, EGF, WNT conditioned media (50%, produced using stably transfected L cells), TGF-b type I receptor inhibitor A83-01 and P38 inhibitor SB202190 as described in [20]. Cells were suspended in 10 μL drops of Matrigel/well, in round bottom 96-well plates.

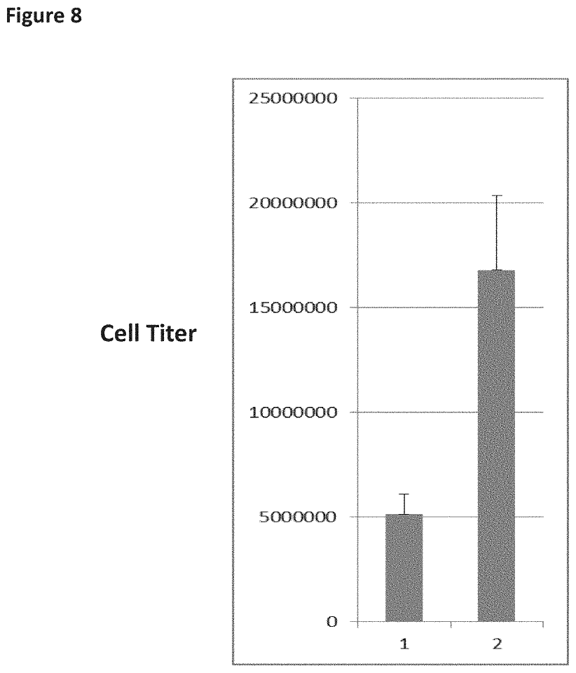

The cells were treated with the medium described above as a control (Bar 1 on FIG. 7) or treated with the same human colon organoid medium supplemented 2 fold dilution of humanised TS2/16 antibody (Bar 2-4 on FIG. 7). The growth of the epithelial stem cells was measured using a CellTiter-Glo® Luminescent Cell Viability assay (Promega) as described above.

The results demonstrate that the addition of this integrin agonist to a culture medium in combination with matrigel-suspension of cells increases epithelial stem cell growth. Therefore, an integrin agonist can improve the growth of epithelial stem cells also in combination with signalling derived from an extracellular matrix.

These results were confirmed in further experiments using Matrigel enriched with Dyna beads. The colon epithelial stem cells were isolated and cultured as above, except the Matrigel used was modified by adding ProtG-coated Dyna beads (Invitrogen/Thermofischer Scientific Ref 10003D) or ProtG-coated Dyna beads saturated with the humanised TS2/16 antibody. FIG. 8 shows the results of the two treatments with (bar 2) or without (bar 1) the humanised TS2/16 antibody. These data also show that the addition of this integrin agonist here presented multivalently through the ProtG-coated Dyna beads, enhances growth of classical matrigel driven epithelial cell growth.

In conclusion, an integrin agonist can stimulate growth of epithelial stem cells or organoids, by adding it as a soluble component to the culture medium, with or without exogenously added extracellular matrix. An integrin agonist can also stimulate growth when incorporated in the extracellular matrix, e.g. when it is cross-linked on a carrier and mixed directly with Matrigel.

### Example 6

### The Effect of HUTS-4 on Culturing Epithelial Stem Cells in Culture Medium Without Any Extracellular Matrix Components

As discussed above, studies have found that the Fab integrin activating antibodies TS2/16, 12G10 and HUTS-4 can induce nearly identical high affinities for cyclic RGD peptide. This observation suggests that these antibodies stabilize identical conformations of the ligand-binding site in the β1 domain. The inventors tested whether the addition of another integrin agonist, HUTS-4, would allow epithelial stem cell growth in a culture medium that does not comprise any exogenously added extracellular matrix components.

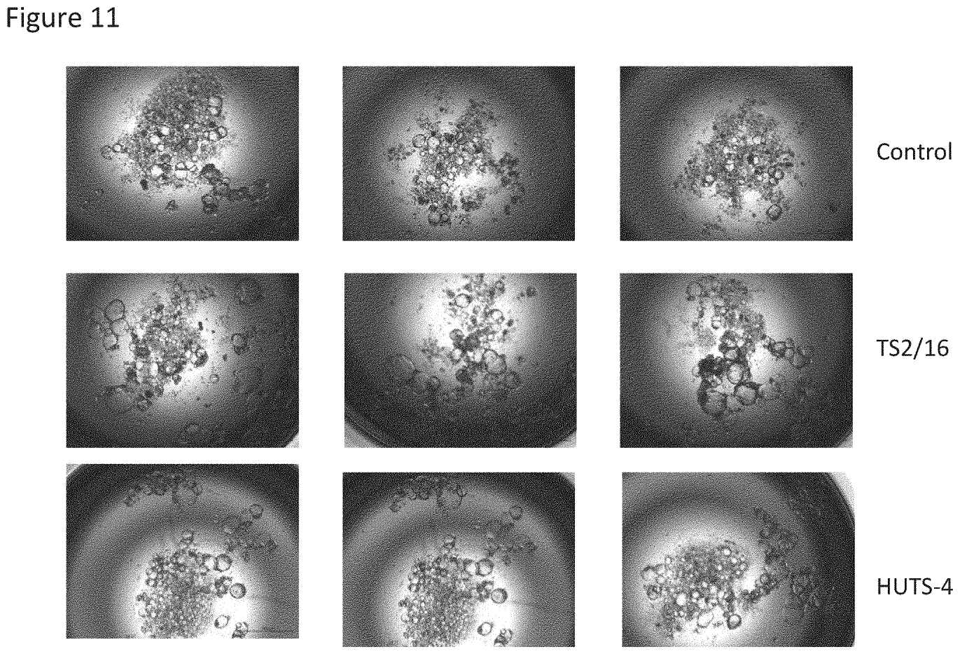

Fragments of human colon organoids were isolated and cultured as described in Example 4 in Corning UltraLowAttachment 96 well flat bottomed plates. These human colon-derived organoid fragments were then cultured in human colon organoid medium supplemented with TS2/16 or HUT-4 at a final concentration of 1 μg/ml, or cultured without an antibody. Three replicates were performed for each treatment condition. The growth rate of the epithelial stem cells was measured on day 7 post treatment by quantifying living cells using the CellTiterGlo ATP-based assay (FIG. 9).

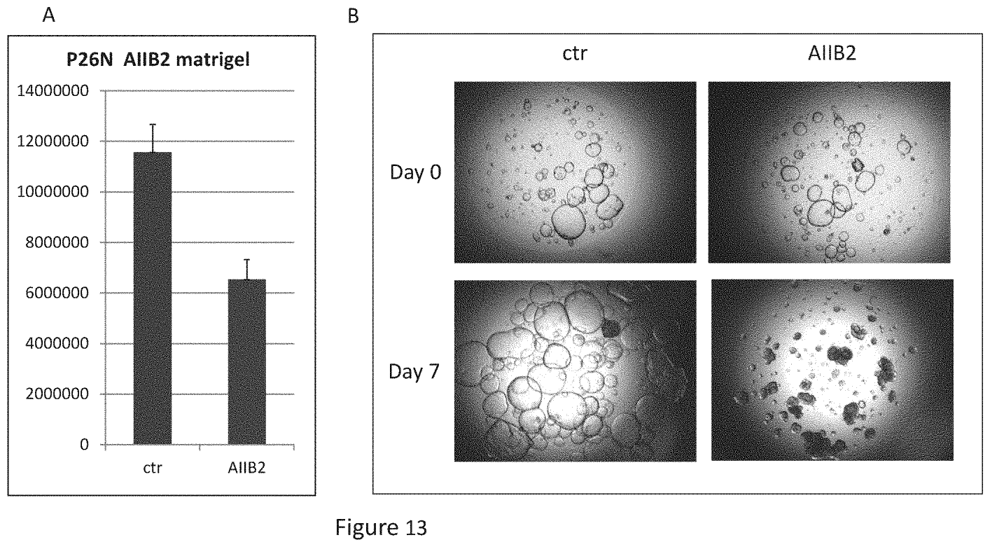

The addition of two different integrin agonists resulted in an increase in epithelial stem cell growth compared to the control culture medium that did not contain an integrin agonist antibody (see FIG. 9). Examples of the growing organoids in all three of the culture media test can be seen in FIG. 11.

The inventors have surprisingly demonstrated that the addition of two different integrin agonists to a culture medium which does not contain any extracellular matrix components can successfully result in efficient epithelial stem cell growth. Therefore, a culture method comprising contracting a cell or organoid with an integrin agonist can be used to improve methods for culturing an epithelial stem cell or an organoid. These data demonstrate that the culture methods described herein can be particularly effective with an integrin agonist has an affinity for cyclic RGD peptide that is similar to the integrin agonists TS2/16, 12G10 and HUTS-4 and/or that can stabilize the same open headpiece conformation of integrin as TS2/16, 12G10 and HUTS-4.

### Example 7

### Human Colon Growth in the Absence of Matrigel Starting from Sheared Organoids

The inventors tested whether an integrin agonist could improve epithelial stem cells growth in a culture medium that does not comprise any exogenously added extracellular matrix components initiate from fragments of an organoid (sheared organoids), rather than single cells as used in Example 4.

The organoid fragments were obtained, mechanically, by repeatedly moving organoids back and forth through a narrow pipette tip. The degree of fragmentation was monitored microscopically. The fragments of human epithelial colon organoids were plated into Corning UltraLowAttachment 96 well flat bottomed plates containing a culture medium as described in Example 4.

The fragments of human epithelial colon organoids ware then cultured in human colon organoid medium supplemented with humanised TS2/16 or without the antibody. Five replicates were performed for each treatment condition. The growth rate of the epithelial stem cells was measured on day 7 post treatment by counting the total number of organoids.

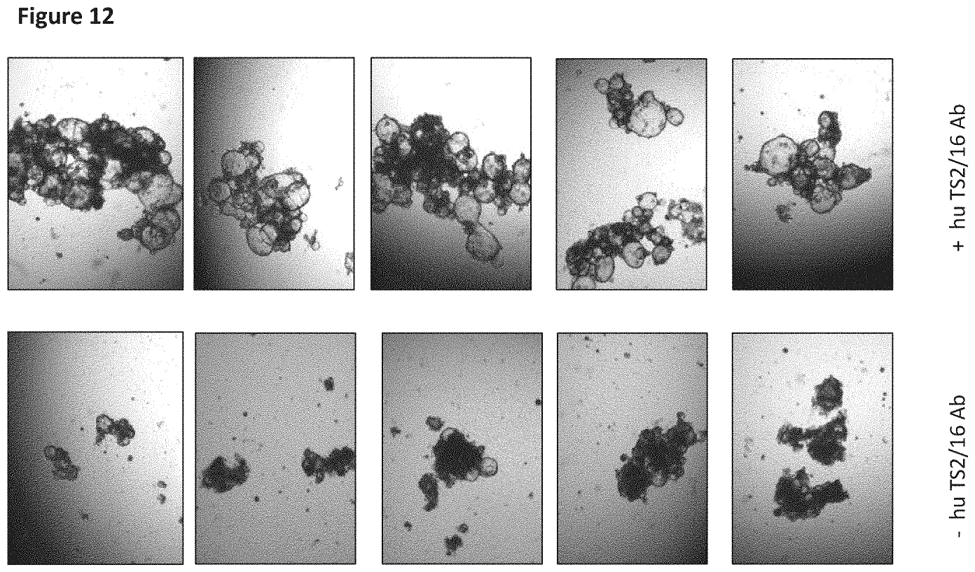

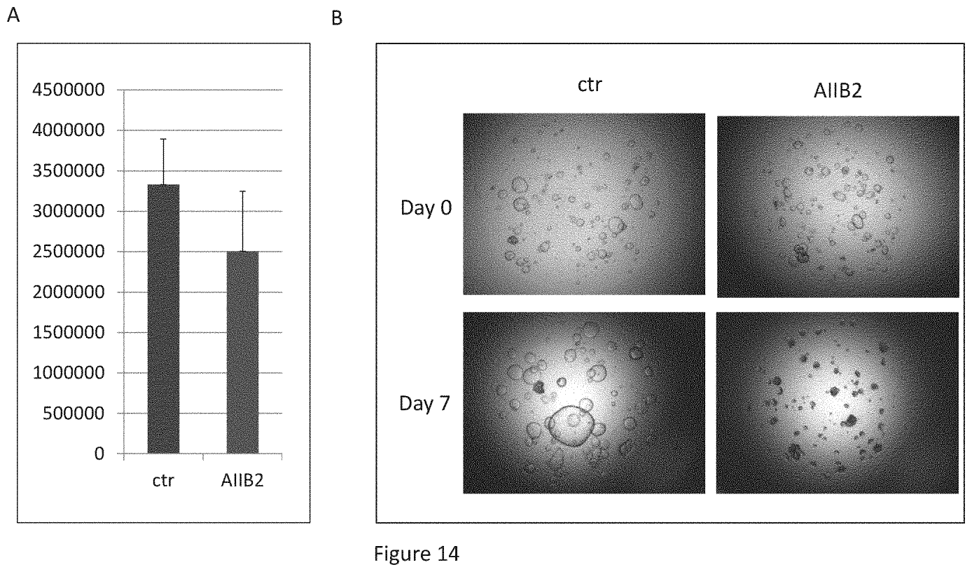

The addition of an integrin agonist to cultures initiated with a fragment of an organoid resulted in increased epithelial stem cell growth compared to the culture medium that did not contain the humanised TS2/16 antibody (see FIG. 10). Examples of growing organoids in the culture medium comprising the humanised TS2/16 antibody and without the antibody can be seen in FIG. 12. There are advantages associated with initiating epithelial stem cell cultures using a fragment of an organoid, rather than a single epithelial stem cell. For example, organoid growth is usually improved in cultures that are initiated with a fragment of an organoid rather than a single cell. Furthermore, single epithelial stem cells are usually obtained by digesting organoids with trypsin, which can cleave integrin receptors from the surface of cells. The cells therefore need time to replace live integrin receptors on their surface before activation by on integrin agonist can occur.

The inventors have surprisingly demonstrated that the addition of an integrin agonist to a culture medium which does not contain any extracellular matrix components can successfully result in efficient epithelial stem cell growth from cultures that have been initiated with both single epithelial stem cell and fragments of organoids.

### Example 8

### Human Colon Growth in the Presence of Matrigel and the Antibody AIIB2

AIIB2 is a known integrin antagonist antibody that can bind the μ1 subunit of integrin. In contrast to the integrin agonist tested in Example 3, AIIB2 has been shown to inhibit cell attachment to the ECM components fibronectin, laminin and collagen IV [21].

Human colon-derived organoid fragments of P26N and STEM159N were isolated as described in Example 6. The human colon-derived organoid fragments were then cultured in the human colon organoid medium described in Example 5. In some of the conditions tested the fragments were suspended in 10 μL drops of Matrigel/well. The following conditions were tested with:
a) P26N organoid fragments in the presence of Matrigel with AIIB2 or without an antibody
b) P26H organoid fragments without Matrigel with AIIB2 or without an antibody; and
c) STEM159N organoid fragments in the presence of Matrigel AIIB2 or without an antibody.
   Two replicates were performed for each treatment condition. The growth rata of the epithelial stem cells was measured on day 7 post treatment by counting the total number of organoids.

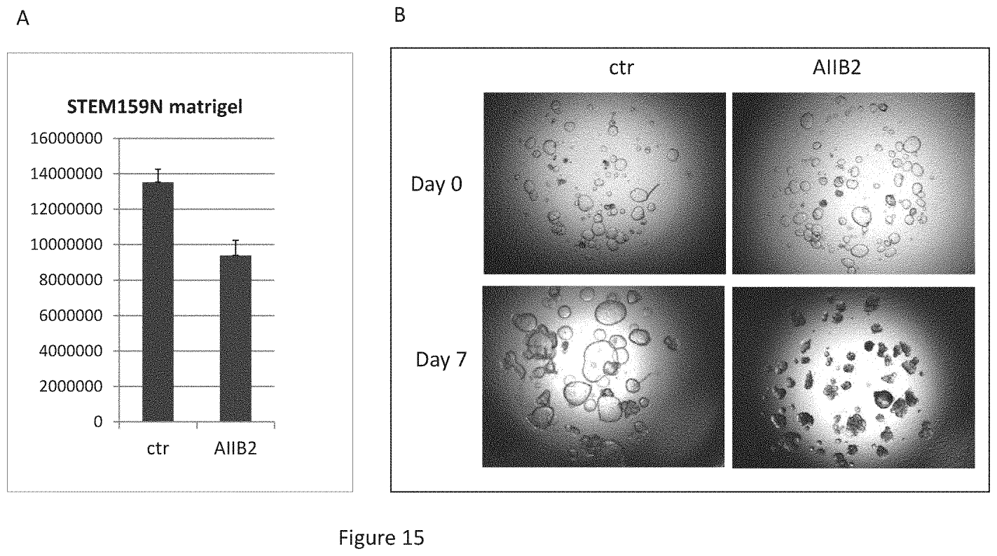

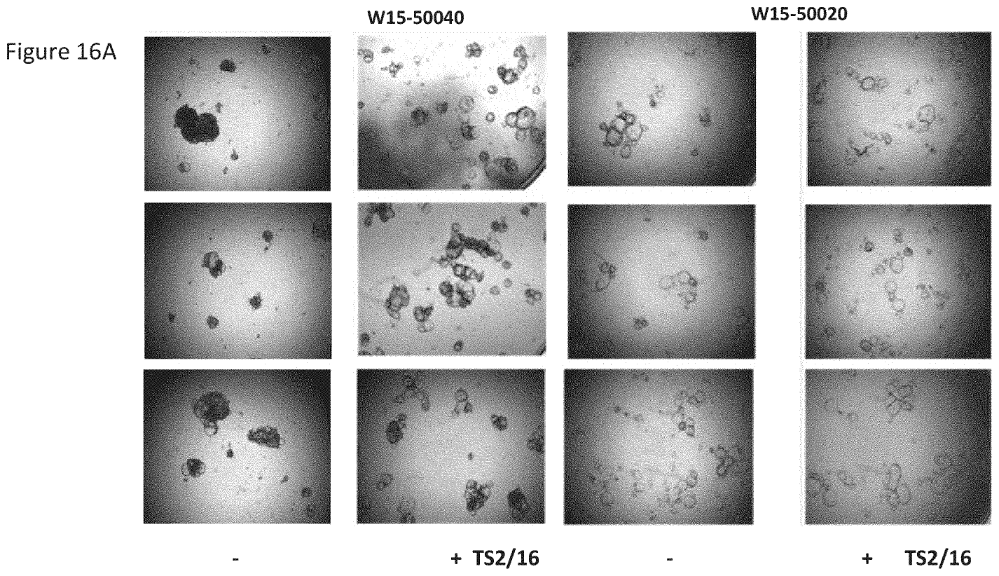

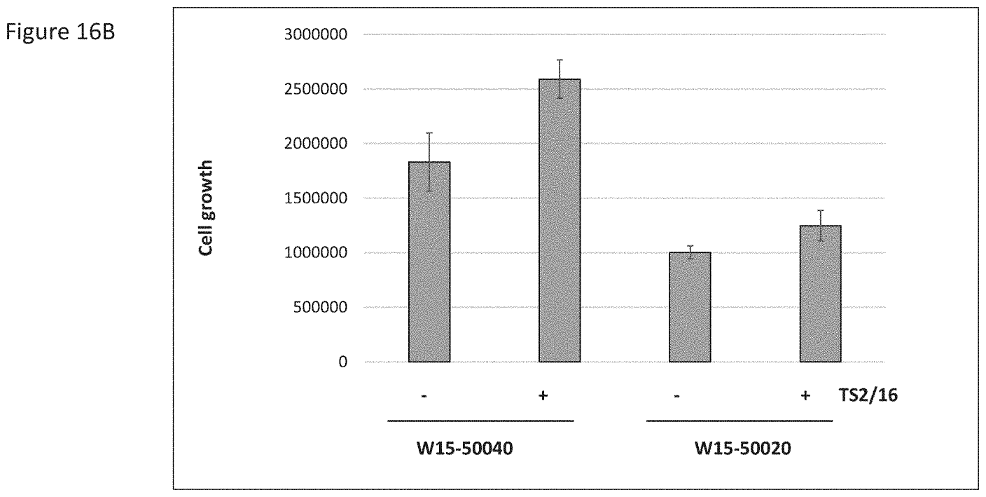

FIGS. 13-15 demonstrate that the presence of AIIB2 in the culture medium prevents epithelial stem cell growth. Without wishing to be bound by any particular theory, this lack of epithelial stem cell growth in the presence of AIIB2 is due to the ability of AIIB2 to block ligands in the Matrigel from activating integrin. Without integrin activation by either ligands in the ECM or an integrin agonist, as described above, the epithelial stem cells do not grow and expand in culture.

Epithelial stem cells in organoids have a basal-OUT phenotype, in which the apical membranes of the cells face the lumen of the organoids. This polarisation is reverted to apical OUT when organoids are growing in the absence of Matrigel due to the lack of a ligand for the Integrin β1 receptor, as described in ref [22]. This apical OUT phenotype is also observed in patients with TTC7A mutations in which integrin-associated ROCK signalling is disturbed [23]. This reversion of polarity can also be induced in matrigel cultures with AIIB2, because the antibody blocks the interaction of the ITGB1 receptor with extracellular matrix components in the Matrigel. This principle of AIIB2 induced reversal of polarity was first shown in MDCK cysts [24].

In summary, the integrin β1 receptor in the extracellular matrix components is essential to ensure the growth of organoids with a basal-OUT phenotype. The inventors have surprisingly demonstrated that the addition of an integrin agonist to a culture medium which does not contain any extracellular matrix components can successfully result in epithelial stem cell growth with a basal-OUT phenotype.

### Example 9

### Culturing Pancreatic Epithelial Stem Cells in Culture Medium Without Any Extracellular Matrix Components

The inventors tested whether an integrin agonist could improve the growth of pancreatic epithelial stem cells in a culture medium that does not comprise any exogenously added extracellular matrix components.

Fragments of two dependent pancreatic organoid lines W15-50040 and W16-50020 were obtained using the method described in Example 6. The pancreatic cells were cultured in AdDMEM/F12 medium supplemented with HEPES (1×), Glutamax (1×), penicilin/streptomycin (1×), B27 (1×), Primocin (1 mg/ml), N-acetyl-L-cysteine (1 mM), Wnt3a-conditioned medium (50% v/v), RSPO1-conditioned medium (10% v/v), Noggin conditioned medium (10% v/v) or recombinant protein (0.1 μg/mL), epidermal growth factor (EGF, 50 ng/ml), Gastrin (10 nM), fibroblast growth factor 10 (FGF10, 100 ng/ml), Nicotinamide (10 mM) and A83-01 (0.5 μM) as described in [25].

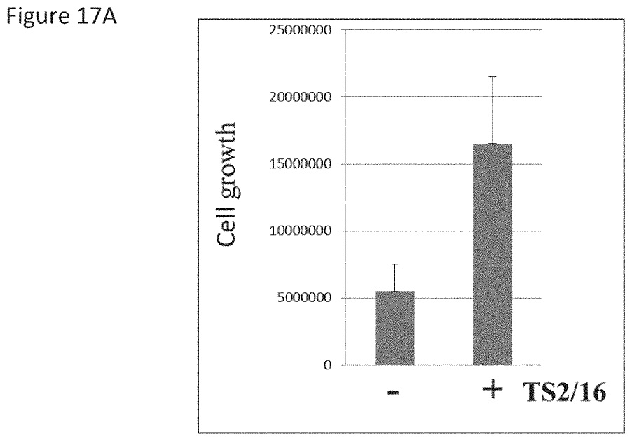

These human pancreas-derived organoid fragments were then cultured in human pancreas organoid medium supplemented with TS2/16 at a final concentration of 1 μg/ml or without an antibody. Three replicates were performed for each treatment condition. The growth rate of the epithelial stem cells was measured on day 6 post treatment by quantifying living cells using the CellTiterGlo ATP-based assay (FIG. 16B). Examples of the growing pancreatic organoids can be seen in FIG. 16A.

The addition of an integrin agonist resulted in an increase in pancreatic epithelial stem cell growth compared to the culture medium that old not contain the humanised TS2/16 antibody (see FIG. 16).

### Example 10

### Culturing Lung Epithelial Stem Cells in Culture Medium Comprising an Extracellular Matrix

The inventors tested whether an integrin agonist could improve the growth of lung epithelial stem cells in a culture medium to a culture medium comprising an extracellular matrix.

Fragments of lung organoid were obtained using the method described in Example 6. The lung organoid fragments were cultured in medium composing advanced DMSM/F12, HEPES, Primocin, Penicillin/Streptomycin, GlutaMax 100×, R-Spondin 1, FGF 7, FGF 10, Noggin, A83-01, Y-27632, SB202190, B27 supplement. N-Acetylcysteine and Nicotinamide as described in [26]. Cells were suspended in 10 μL drops of Matrigel/well, in round bottom 96-well plates.

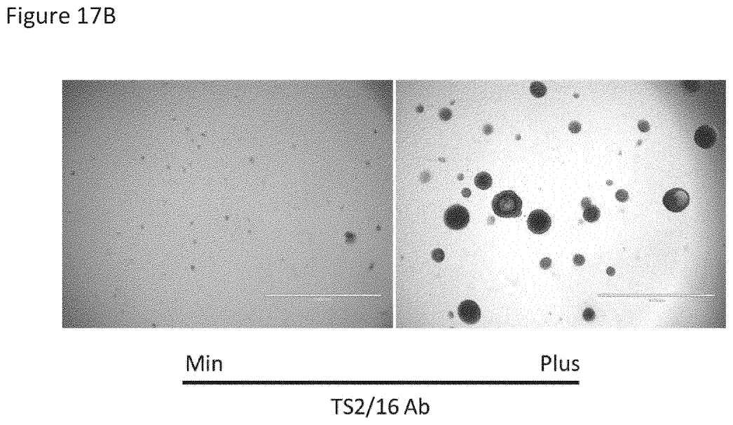

These human lung-derived organoid fragments were then cultured in human lung organoid medium supplemented with TS2/16 at a final concentration of 1 μg/ml or without an antibody. Three replicates were performed for each treatment condition. The growth rate of the epithelial stem cells was measured on day 12 post treatment by quantifying living cells using the CellTiterGlo ATP-based assay (FIG. 17A). Examples of the growing lung organoids can be seen in FIG. 17B.

The addition of an integrin agonist resulted in an increase in lung epithelial stem cell growth compared to the culture medium that did not contain the humanised TS2/16 antibody (see FIG. 17).

### Example 11

### Culturing Head and Neck Epithelial Stem Cells in Culture Medium Comprising an Extracellular Matrix

The inventors tested whether an integrin agonist could improve the growth of head and neck epithelial stem cells in culture medium that does not comprise my exogenously added extracellular matrix components.

Fragments of head and neck organoid were obtained using the method described in Example 6. The head and neck organoid fragments were cultured in medium comprising Advanced DMEM +/+/+ with 1×B27 supplement, 1 mM N-acetyl-L-cysteine, 10 mM Nicotinamide 505, 50 ng/ml human EGF, 500 nM A83-01, 10 ng/ml human FGF 10, 5 ng/ml human FGF2, 1 μM Prostaglandin E2, 3 μM CHIR-99021, 1 μM Forskolin, 4% R-spondin and 4% Noggin. Cells were suspended in 10 μL drops of Matrigel/well, in round bottom 96-well plates.

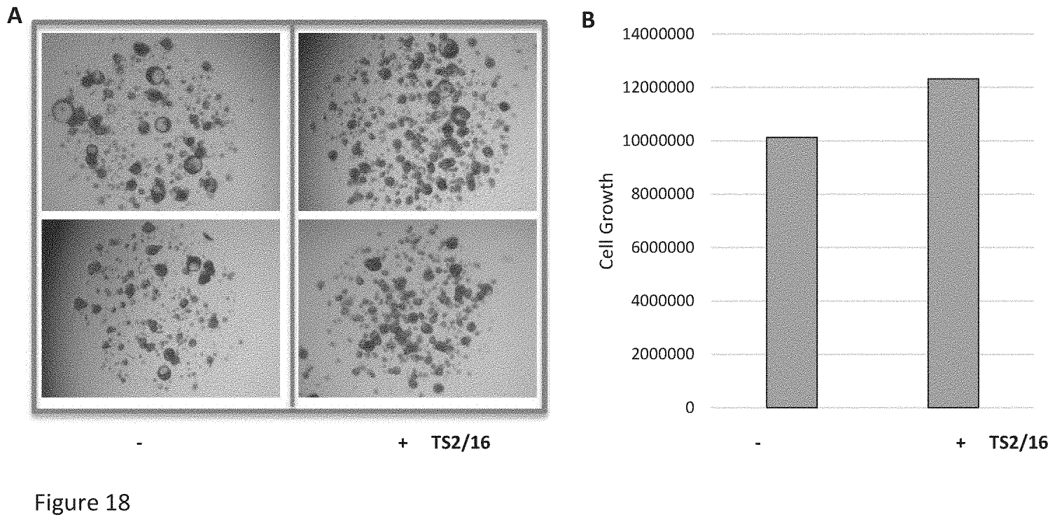

These human head and neck-derived organoid fragments were then cultured in human head and neck organoid medium supplemented with TS2/16 at a final concentration of 1 μg/ml or without an antibody. Three replicates were performed for each treatment condition. The growth rate of the epithelial stem cells was measured on day 7 post treatment by quantifying living cells using the CellTiterGlo ATP-based assay (FIG. 18B). Examples of the growing head and neck organoids can be seen in FIG. 18A. The addition of an integrin agonist resulted in an increase in head and neck epithelial stem cell growth compared to the culture medium that did not contain the humanised TS1/16 antibody (see FIG. 18).

### Example 12

### Human Colon Growth in the Presence of the Antibody Asc8

Asc8 is a known integrin antagonist antibody that binds to the β4 subunit of integrin Asc8 blocks cell adhesion and has been shown to inhibit wound closure [27].

Colon epithelial stem cells were isolated as described in Example 4 and cultured in a medium according to Example 5. Cells were suspended in 10 μL drops of Matrigel/well, in round bottom 96-well plates. The cells were treated with the medium described above as a control or treated with:
(a) A final concentration of 0.3 μg/ml of Asc8.
(b) A final concentration of 1.2 μg/ml of Asc8.
(c) A final concentration of 5 μg/ml of Asc8.
(d) A final concentration of 1 μg/ml of TS2/16.

The growth of the epithelial stem cells was measured using a CellTiter-Glo® Luminescent Cell Viability assay (Promega) as described above.

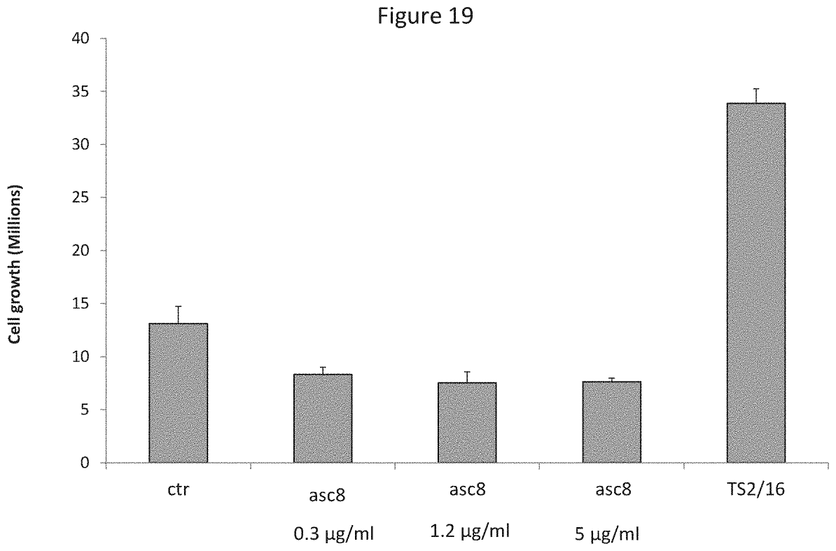

FIG. 19 demonstrates that the presence of Asc8 to the culture medium prevents epithelial stem cell growth. This observation is the similar to that observed with AIIB2 described in Example 8. Again, without wishing to be bound by any particular theory, this lack of epithelial stem cell growth in the presence of Asc8 is thought to be due to the ability of this antibody to block ligands in the Matrigel from activating integrin.

### Example 13

### Synergistic Effect of Multiple Integrin Agonists

3E1 is a β4 integrin agonist antibody. Integrin β4 engagement of either its natural ligand or an activating antibody leads to tyrosine phosphorylation of the β4 cytoplasmic, which regulates multiple signalling pathways important for tumour development [28]. It has been shown that phosphorylated Src associates with integrin β4 in A431 cells in response to laminin or 3E1 stimulation which increases Src kinase activity.

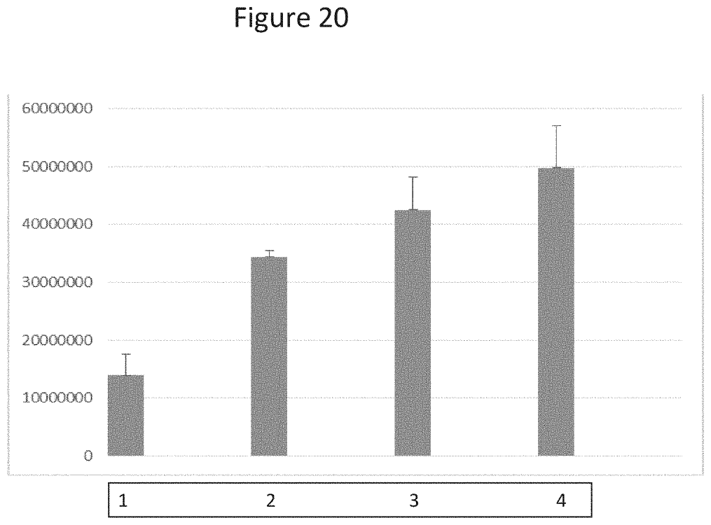

3E1 was obtained from the Memorial Sloan Kettering Cancer Centre Antibody & Bioresource Core Facility. The antibody is also sold by Sigma Aldrich under catalogue number MAB1964. Colon epithelial stem cells were isolated as described in Example 4 and cultured in a medium according to Example 8. Cells were suspended in 10 μL drops of Matrigel/well, in round bottom 96-well plates. The cells were treated with the medium described above as a control (bar 1) or treated with:
(a) A final concentration of 0.1 μg/ml of TS2/16 (bar 2).
(b) A final concentration of 0.1 μg/ml of TS2/16 and 0.1 μg/ml of 3E1 (bar 3).
(c) A final concentration of 0.1 μg/ml of TS2/16 and 1 μg/ml of 3E1 (bar 4).
   The growth of the epithelial stem cells was measured using a CellTiter-Glo® Luminescent Cell Viability assay (Promega) as described above. FIG. 20 demonstrates the synergistic effect of the β1 integrin agonist TS/16 and the β4 integrin agonist 3E1 on epithelial stem cell growth. Therefore, multiple integrin agonists can improve the growth of epithelial stem cells in combination with signalling derived from an extracellular matrix.

The data presented in the examples above demonstrate the surprising ability of an integrin agonist to successfully achieve efficient epithelial stem cell growth in a variety of cell types in the presence and absence of extracellular matrix components.

SEQUENCES

SEQ ID NO: 1-TS2/16 H-chain
MGWSCIILFLVATATGVHSMDVKLVESGGGLVKPGGSLKLSCAASGFTF
SSYTMSWVRQTPEKRLEWVATISSGGSYTYYPDSVKGRFTISRDKAKNT
LYLQMGSLKSEDTAMYYCTRIGYDEDYAMDHWGQGTSVTVSSASTKGPS
VFPLAPSSKSTSGGTAALGC,LVKDYFPEPVTVSWNSGALTSGVHTFPA
VLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRW
QQGNVFSCSVMHEALHNHYTQKSLSLSPGK*
SEQ ID NO: 2-TS2-16 kL-chain
MGWSCIILFLVATATGVHSEIVVTQSPTTMAASPGDKITITCSVSSIIS
SNYLHWYSQKPGFSPKLLIYRTSNLASGVPPRFSGSGSGTSYSLTIGTM
EAEDVATYYCQQGSDIPLTFGDGTKLDLKRTVAAPSVFIFPPSDEQLKS
GTASVVCLLNNFYPREAKVQWKVDNALQSGNSQESVTEQDSKDSTYSLS
STLTLSKADYEKHKVYACEVTHQGLSSPVTKSFNRGEC
SEQ ID NO: 3-Humanised TS2/16 H-chain
MDVKLVESGGGLVKPGGSLKLSCAASGFTFSSYTMSWVRQTPEKRLEWV
ATISSGGSYTYYPDSVKGRFTISRDKAKNTLYLQMGSLKSEDTAMYYCT
RIGYDEDYAMDHWGQGTSVTVSS
SEQ ID NO: 4-Humanised TS2/16 L-chain
EIVTQSPTTMAASPGDKITITCSVSSIISSNYLHWYSQKPGFSPKLLIY
RTSNLASGVPPRFSGSGSGTSYSLTIGTMEAEDVATYYCQQGSDIPLTF
GDGTKLDLK
SEQ ID NO: 5-Humanised TS2/16 H-chain HCDR1:
GFTFSSYTMS
SEQ ID NO: 6-Humanised TS2/16 H-chain HCDR2:
TISSGGSYTYYPDSVKG
SEQ ID NO: 7-Humanised TS2/16 H-chain HCDR3:
IGYDEDYAMDH
SEQ ID NO: 8-Humanised TS2/16 L-chain LCDR1:
SVSSIISSNYLH
SEQ ID NO: 9-Humanised TS2/16 L-chain LCDR2:
RTSNLAS
SEQ ID NO: 10-Humanised TS2/16 L-chain LCDR3:
QQGSDIPLT

### REFERENCES

[1] Ahmed and Constant Curr Stem Cell Rep. 2016; 2(3): 197-206.

[2] Hughes et al. (2010) Proteomics 10(9):1836-90

[3] Byron et al. (2000) Journal of Cell Science 122, 4009-4011

[4] Humphries et al (2006) Journal of Cell Science 119; 3901-3903

[5] Humphries (2000) Biochemical Society Transactions vol. 28, part 4

[6] Su et al. (2016) PNAS 113 (27) E3872-E3881

[7] Li et al. (2017) EMBO 36.629-645

[8] Sun et al. (2019) Nature Cell Biology 21, 25-31

[9] Calderwood (2004) Journal of Cell Science 117 (5)

[10] Pokharel et al. (2019) Nature communications, https://doi.org/10.1039/s41467-019-09453-x

[11] Shattil et al. (2010) Nature reviews, 11, 298-300

[12] Luque et al. (1996) J. Bio Chem. 271, 19, 11057-11075

[13] Tsuchida et al. (1997) FEBS Letters 416, 212-21

[14] Hughes et al. (2010) Proteomics 10(9): 1886-90

[15] Gjorevski et al. (2016) Nature, 539, 560-564

[16] Cruz-Acuña et al. (2017) Nat Cell Biol. (11): 1326 -1335

[17] Tsuchida et al. (1992) FEBS Letters 416 212-216

[18] M van de Watering et al. (2015) Cell; 161 (4):933-45

[19] Janda et al. (2017) Nature, 545(7653):234-237

[20] Drost et al. (2015) Nature 521: 43-47

[21] Hall et al. (1990) The Journal of Cell Biology, 110, 2175-2184

[22] Co et al. (2019) Cell Reports 26, 2509-2520

[23] Bigorgne et al. (2014) J Clin Invest. 124, 328-337

[24] Yu et al. (2005) Molecular Biology of the cell 16, 433-445

[25] Boh et al. (2015) Cell 160(1-2):324-38

[26] Sachs et al. (2019) EMBO 38(4):e100300. doi:10.15252/embj.201810030

[27] Egles et al. (2010) PlosOne, 5(5); e10528.

[28] Dutta et al. (2000) Cancer Res.68:8779-8787

1. A method for culturing an epithelial stem cell or an organoid comprising epithelial stem cells, wherein the method comprises culturing said epithelial stem cell in a culture medium suitable for epithelial stem cells, wherein the culture method further comprises contacting the cell or organoid with an integrin agonist, and wherein an organoid is obtained or the method results in the growth of the organoid.

2. The method of claim 1, wherein
(a) the integrin agonist interacts with the beta subunit of integrin, optionally wherein the beta subunit is β1, β2, β3 or β7, further optionally wherein the integrin agonist interacts with the β1 subunit; or
(b) the integrin agonist interacts with the alpha subunit of integrin.

3-4. (canceled)

5. The method of claim 1, wherein the integrin agonist is selected from an anti-integrin antibody, a talin, a kindlin, dithiothreitol and oxysterol 25-hydroxycholesterol.

6. The method of claim 1, wherein the integrin agonist is
(a) an anti-integrin antibody, optionally wherein the anti-integrin antibody is JBS2, HP1/3, SNAKA51, PT25-2, PMI-1, MEM-83, NKI-L16, 496B, 12G10, 8A2, TS2/16, 15/7, HUTS-4, 8E3, N29, 9EG7, mAb 24, MEM-148, KIM127, CBR LFA-1/2, MEM-48, KIM185, AP3, APS, LIBS6, LIBS2, 10F8, 2B8, 2G3;
(b) a talin, optionally, wherein the talin is used in combination with a kindlin;
(c) a reducing agent, such as dithiothreitol; or
(d) a lipid, such as oxysterol 25-hydroxycholesterol.

7. The method of claim 1, wherein the integrin agonist is an anti-integrin antibody, and wherein the anti-integrin antibody is TS2/16, 12G10, 8A2, 15/7, HUTS-4, 8E3, N29 or 9EG7, optionally wherein the anti-integrin antibody is TS2/16, 12G10, HUTS-4 or 8A2, further optionally wherein the anti-integrin antibody is TS2/16, 12G10, HUTS-4.

8. The method of claim 5, wherein the anti-integrin antibody is humanised.

9. (canceled)

10. The method of claim 1, wherein
(a) at least a 10%, 20%, 50% increase in epithelial stem cell growth is observed over 4 days relative to the same method performed without the integrin agonist; and/or
(b) the method is feeder cell-free.

11. The method of claim 1, wherein the method further comprises culturing the cells in contact with an extracellular matrix, optionally wherein the extracellular matrix is a Basement Membrane Extract or Matrigel.

12. The method of claim 1, wherein the method further comprises culturing the cells in contact with a synthetic matrix, optionally wherein the synthetic matrix comprises a polymer, optionally a polyester, polyethylene glycol or a hydrogel, optionally wherein the synthetic matrix comprises:
(a) a cross-linked polyethylene glycol (PEG) hydrogel; and/or
(b) a biomaterial, preferably an extracellular matrix component, optionally wherein the biomaterial is one or more glycoprotein (optionally selected from collagen, laminin, perlecan, fibronectin or an RGD attachment ligand of fibronectin) and/or one or more carbohydrate (optionally hyaluronic acid).

13. (canceled)

14. The method of claim 1, wherein the method further comprises culturing the cells in contact with an extracellular matrix or a synthetic matrix, wherein the extracellular matrix or synthetic matrix is three-dimensional and/or is in suspension.

15. The method of claim 1, wherein the culture method does not involve contacting the cells with an exogenous extracellular matrix.

16. The method of claim 1, wherein the epithelial stem cell is selected from a colorectal, small intestine, stomach, pancreas, liver, lung, breast, prostate, kidney, mouth, nasopharynx, throat, hypopharynx, larynx, trachea, skin, fallopian tube, ovary, salivary gland, esophagus, hair follicle and/or cochlear cell.

17. The method of claim 1, wherein the culture medium suitable for epithelial stem cells comprises one or more of a Wnt agonist, a BMP inhibitor, a mitogenic growth factor and a TGF-beta inhibitor.

18. A culture medium suitable for generating an organoid or maintaining an organoid as defined in claim 17, wherein the culture medium further comprises an integrin agonist wherein
(a) the integrin agonist interacts with the beta subunit of integrin, optionally wherein the beta subunit is β1, β2, β3 or β7, further optionally wherein the integrin agonist interacts with the β1 subunit; or
(b) the integrin agonist interacts with the alpha subunit of integrin; or
(c) the integrin agonist is an anti-integrin antibody, optionally wherein the anti-integrin antibody is JBS2, HP1/3, SNAKA51, PT25-2, PMI-1, MEM-83, NKI-L16, 496B, 12G10, 8A2, TS2/16, 15/7, HUTS-4, 8E3, N29, 9EG7, mAb 24, MEM-148, KIM127, CBR LFA-1/2, MEM-48, KIM185, AP3, AP5, LIBS6, LIBS2, 10F8, 2B8, 2G3; or
(d) the integrin agonist is talin, optionally, wherein the talin is used in combination with a kindlin; or
(e) the integrin agonist is a reducing agent, such as dithiothreitol; or
(f) the integrin agonist is a lipid, such as oxysterol 25-hydroxycholesterol.

19-20. (canceled)

21. A composition comprising a culture medium suitable for generating an organoid or maintaining an organoid as defined in claim 18, and an extracellular matrix or a synthetic matrix.

22. Use of an integrin agonist for culturing an epithelial stem cell to generate an organoid or for maintaining an organoid, optionally wherein
(a) the integrin agonist interacts with the beta subunit of integrin, optionally wherein the beta subunit is β1, β2, β3 or β7, further optionally wherein the integrin agonist interacts with the β1 subunit; or
(b) the integrin agonist interacts with the alpha subunit of integrin; or
(c) the integrin agonist is an anti-integrin antibody, optionally wherein the anti-integrin antibody is JBS2, HP1/3, SNAKA51, PT25-2, PMI-1, MEM-83, NKI-L16, 496B, 12G10, 8A2, TS2/16, 15/7, HUTS-4, 8E3, N29, 9EG7, mAb 24, MEM-148, KIM127, CBR LFA-1/2, MEM-48, KIM185, AP3, APS, LIBS6, LIBS2, 10F8, 2B8, 2G3; or
(d) the integrin agonist is talin, optionally, wherein the talin is used in combination with a kindlin; or
(e) the integrin agonist is a reducing agent, such as dithiothreitol; or
(f) the integrin agonist is a lipid, such as oxysterol 25-hydroxycholesterol.

23-25. (canceled)

26. The composition of claim 21, wherein
(a) the extracellular matrix is a Basement Membrane Extract or Matrigel,
(b) the synthetic matrix comprises a polymer, optionally a polyester, polyethylene glycol or a hydrogel, optionally wherein the synthetic matrix comprises:
i. a cross-linked polyethylene glycol (PEG) hydrogel; and/or
ii. a biomaterial, preferably an extracellular matrix component, optionally wherein the biomaterial is one or more glycoprotein (optionally selected from collagen, laminin, perlecan, fibronectin or an RGD attachment ligand of fibronectin) and/or one or more carbohydrate (optionally hyaluronic acid).
